SEQUENCE LISTING (70 sequences)

SEQ ID NO: 1 - DNA, 48 bp, Artificial Sequence
  primer
tgtcttgaatccaagatgaagcgcgcccgccccagcgaagatgacttc

SEQ ID NO: 2 - DNA, 48 bp, Artificial Sequence
  primer
tggagctggtgtggtccacaaagtgcgcgtgtcatattctgggttcca

SEQ ID NO: 3 - DNA, 24 bp, Artificial Sequence
  primer
actttgtggaccacaccagctcca

SEQ ID NO: 4 - DNA, 30 bp, Artificial Sequence
  primer
cataacgcggccgcttctttattcttgggc

SEQ ID NO: 5 - DNA, 42 bp, Artificial Sequence
  primer
gtgctactaaacaattccttcctggatccagaatattggaac

SEQ ID NO: 6 - DNA, 42 bp, Artificial Sequence
  primer
gttccaatattctggatccaggaaggaattgtttagtagcac

SEQ ID NO: 7 - DNA, 30 bp, Artificial Sequence
  primer
atgggatccaagatgaagcgcgcaagaccg

SEQ ID NO: 8 - DNA, 30 bp, Artificial Sequence
  primer
tggtgtggtccacaaagttagcttatcatt

SEQ ID NO: 9 - DNA, 48 bp, Artificial Sequence
  primer
aagctaactttgtggaccacaccagacacatctccaaactgcacaatt

SEQ ID NO: 10 - DNA, 28 bp, Artificial Sequence
  primer
aaacacggcggccgctctttcattcttg

SEQ ID NO: 11 - DNA, 45 bp, Artificial Sequence
  primer
ctttgtggaccacaccagacactagtccaaactgcacaattgctc

SEQ ID NO: 12 - DNA, 45 bp, Artificial Sequence
  primer
gagcaattgtgcagtttggactagtgtctggtgtggtccacaaag

SEQ ID NO: 13 - DNA, 48 bp, Artificial Sequence
  primer
gcttaggttaacctcaagctttttcttggtttttttgagaggtgggct

SEQ ID NO: 14 - DNA, 48 bp, Artificial Sequence
  primer
agcccacctctcaaaaaaaccaggaaaaagcttgaggttaacctaagc

SEQ ID NO: 15 - DNA, 72 bp, Artificial Sequence
  primer
atcagtattaacttgcagtggagccttagggtttacagttaggcttccggcctcgtccagagagaggccgtt

SEQ ID NO: 16 - DNA, 72 bp, Artificial Sequence
  primer
ggaagcctaactgtaaaccctaaggctccactgcaagttaatactgattcaaacataaacctggaaatatct

SEQ ID NO: 17 - DNA, 72 bp, Artificial Sequence
  primer
atcattgtcaaatgtcaacccttctcttgctcttacatttataccaatgttgtaatcaaattctaggccatg

SEQ ID NO: 18 - DNA, 72 bp, Artificial Sequence
  primer
attggtataaatgtaagagcaagagaagggttgacatttgacaatgatggtgccattacagtaggaaacaaa

SEQ ID NO: 19 - DNA, 38 bp, Artificial Sequence
  primer
ctggacgaggccggcagcctaactgtaaaccctaaggc

SEQ ID NO: 20 - DNA, 38 bp, Artificial Sequence
  primer
gccttagggtttacagttaggctgccggcctcgtccag

SEQ ID NO: 21 - DNA, 7960 bp, Artificial Sequence
  pDV67
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaaagaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 22 - DNA, 1240 bp, Artificial Sequence
  Adenovirus TPL
ggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatct

SEQ ID NO: 23 - DNA, 7607 bp, Artificial Sequence
  Plasmid GRE5-2.E1
tctagaagatccgctgtacaggatgttctagctactttattagatccgctgtacaggatgttctagctactttattagatccgctgtacaggatgttctagctactttattagatccgctgtacaggatgttctagctactttattagatccgtgtacaggatgttctagctactttattagatcgatctcctggccgttcggggtcaaaaaccaggtttggctataaaagggggtgggggcgcgttcgtcctcactctcttccgcatcgctgtctgcgagggccaggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattagatctaagcttctgcagctcgaggactcggtcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctcagctgactgcttaagtcgcaagccgaattggatccaattcggatcgatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattaattcgggg

SEQ ID NO: 24 - DNA, 11600 bp, Artificial Sequence
  Plasmid pMNNeoE2-a-3.1
gaattccgcattgcagagatattgtatttaagtgcctagctcgatacaataaacgccatttgaccattcaccacattggtgtgcacctccaagcttgggcagaaatggttgaactcccgagagtgtcctacacctaggggagaagcagccaaggggttgtttcccaccaaggacgacccgtctgcgcacaaacggatgagcccatcagacaaagacatattcattctctgctgcaaacttggcatagctctgctttgcctggggctattgggggaagttgcggttcgtgctcgcagggctctcacccttgactcttttaatagctcttctgtgcaagattacaatctaaacaattcggagaactcgaccttcctcctgaggcaaggaccacagccaacttcctcttacaagccgcatcgattttgtccttcagaaatagaaataagaatgcttgctaaaaattatatttttaccaataagaccaatccaataggtagattattagttactatgttaagaaatgaatcattatcttttagtactatttttactcaaattcagaagttagaaatgggaatagaaaatagaaagagacgctcaacctcaattgaagaacaggtgcaaggactattgaccacaggcctagaagtaaaaaagggaaaaaagagtgtttttgtcaaaataggagacaggtggtggcaaccagggacttataggggaccttacatctacagaccaacagatgcccccttaccatatacaggaagatatgacttaaattgggataggtgggttacagtcaatggctataaagtgttatatagatccctcccttttcgtgaaagactcgccagagctagacctccttggtgtatgttgtctcaagaagaaaaagacgacatgaaacaacaggtacatgattatatttatctaggaacaggaatgcacttttggggaaagattttccataccaaggaggggacagtggctggactaatagaacattattctgcaaaaactcatggcatgagttattatgaatagcctttattggcccaaccttgcggttcccagggcttaagtaagtttttggttacaaactgttcttaaaacgaggatgtgagacaagtggtttcctgacttggtttggtatcaaaggttctgatctgagctctgagtgttctattttcctatgttcttttggaatttatccaaatcttatgtaaatgcttatgtaaaccaagatataaaagagtgctgattttttgagtaaacttgcaacagtcctaacattcacctcttgtgtgtttgtgtctgttcgccatcccgtctccgctcgtcacttatccttcactttccagagggtccccccgcagaccccggcgaccctcaggtcggccgactgcggcagctggcgcccgaacagggaccctcggataagtgacccttgtctctatttctactatttggtgtttgtcttgtattgtctctttcttgtctggctatcatcacaagagcggaacggactcaccatagggaccaagctagcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggccggtgcacacggaccacgtcaaagacttcaaacaaaacataaagaagggtgggctcgtccatgggatccatatatagggcccgggttataattacctcaggtcgacctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccact

SEQ ID NO: 25 - DNA, 24 bp, Artificial Sequence
  primer
tgtacaccggatccggcgcacacc

SEQ ID NO: 26 - DNA, 35 bp, Artificial Sequence
  primer
cacaacgagctcaattaattaattgccacatcctc

SEQ ID NO: 27 - DNA, 26 bp, Artificial Sequence
  primer
cgcgctgactcttaaggactagtttc

SEQ ID NO: 28 - DNA, 37 bp, Artificial Sequence
  primer
gcgcttaattaacatcatcaataatataccttatttt

SEQ ID NO: 29 - DNA, 24 bp, Artificial Sequence
  primer
atcatggccgacaagcagaagaac

SEQ ID NO: 30 - DNA, 24 bp, Artificial Sequence
  primer
gtacagctcgtccatgccgagagt

SEQ ID NO: 31 - DNA, 26 bp, Artificial Sequence
  EGFP probe
  misc_feature 1: FAM (6-carboxy-fluorescein)
  misc_feature 26: TAMRA (6-carboxy-tetramethyl-rhodamine)
caggaccatgtgatcgcgcttctcgt

SEQ ID NO: 32 - DNA, 1749 bp, Adenovirus serotype 2 fiber
  CDS (1)...(1749)
atgaaacgcgccagaccgtctgaagacaccttcaaccccgtgtatccatatgacacagaaaccgggcctccaactgtgccctttcttacccctccatttgtttcacccaatggtttccaagaaagtccccctggagttctctctctacgcgtctccgaacctttggacacctcccacggcatgcttgcgcttaaaatgggcagcggtcttaccctagacaaggccggaaacctcacctcccaaaatgtaaccactgttactcagccacttaaaaaaacaaagtcaaacataagtttggacacctccgcaccacttacaattacctcaggcgccctaacagtggcaaccaccgctcctctgatagttactagcggcgctcttagcgtacagtcacaagccccactgaccgtgcaagactccaaactaagcattgctactaaagggcccattacagtgtcagatggaaagctagccctgcaaacatcagcccccctctctggcagtgacagcgacacccttactgtaactgcatcacccccgctaactactgccacgggtagcttgggcattaacatggaagatcctatttatgtaaataatggaaaaataggaattaaaataagcggtcctttgcaagtagcacaaaactccgatacactaacagtagttactggaccaggtgtcaccgttgaacaaaactcccttagaaccaaagttgcaggagctattggttatgattcatcaaacaacatggaaattaaaacgggcggtggcatgcgtataaataacaacttgttaattctagatgtggattacccatttgatgctcaaacaaaactacgtcttaaactggggcagggacccctgtatattaatgcatctcataacttggacataaactataacagaggcctatacctttttaatgcatcaaacaatactaaaaaactggaagttagcataaaaaaatccagtggactaaactttgataatactgccatagctataaatgcaggaaagggtctggagtttgatacaaacacatctgagtctccagatatcaacccaataaaaactaaaattggctctggcattgattacaatgaaaacggtgccatgattactaaacttggagcgggtttaagctttgacaactcaggggccattacaataggaaacaaaaatgatgacaaacttaccctgtggacaaccccagacccatctcctaactgcagaattcattcagataatgactgcaaatttactttggttcttacaaaatgtgggagtcaagtactagctactgtagctgctttggctgtatctggagatctttcatccatgacaggcaccgttgcaagtgttagtatattccttagatttgaccaaaacggtgttctaatggagaactcctcacttaaaaaacattactggaactttagaaatgggaactcaactaatgcaaatccatacacaaatgcagttggatttatgcctaaccttctagcctatccaaaaacccaaagtcaaactgctaaaaataacattgtcagtcaagtttacttgcatggtgataaaactaaacctatgatacttaccattacacttaatggcactagtgaatccacagaaactagcgaggtaagcacttactctatgtcttttacatggtcctgggaaagtggaaaatacaccactgaaacttttgctaccaactcttacaccttctcctacattgcccaggaataa

SEQ ID NO: 33 - protein, 582 aa, Adenovirus serotype 2 fiber
MKRARPSEDTFNPVYPYDTETGPPTVPFLTPPFVSPNGFQESPPGVLSLRVSEPLDTSHGMLALKMGSGLTLDKAGNLTSQNVTTVTQPLKKTKSNISLDTSAPLTITSGALTVATTAPLIVTSGALSVQSQAPLTVQDSKLSIATKGPITVSDGKLALQTSAPLSGSDSDTLTVTASPPLTTATGSLGINMEDPIYVNNGKIGIKISGPLQVAQNSDTLTVVTGPGVTVEQNSLRTKVAGAIGYDSSNNMEIKTGGGMRINNNLLILDVDYPFDAQTKLRLKLGQGPLYINASHNLDINYNRGLYLFNASNNTKKLEVSIKKSSGLNFDNTAIAINAGKGLEFDTNTSESPDINPIKTKIGSGIDYNENGAMITKLGAGLSFDNSGAITIGNKNDDKLTLWTTPDPSPNCRIHSDNDCKFTLVLTKCGSQVLATVAALAVSGDLSSMTGTVASVSIFLRFDQNGVLMENSSLKKHYWNFRNGNSTNANPYTNAVGFMPNLLAYPKTQSQTAKNNIVSQVYLHGDKTKPMILTITLNGTSESTETSEVSTYSMSFTWSWESGKYTTETFATNSYTFSYIAQE

SEQ ID NO: 34 - DNA, 1746 bp, Adenovirus serotype 5 fiber
  CDS (1)...(1746)
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataa

SEQ ID NO: 35 - protein, 581 aa, Adenovirus serotype 5 fiber
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 36 - DNA, 1098 bp, Adenovirus serotype 37 fiber
  CDS (1)...(1098)
atgtcaaagaggctccgggtggaagatgacttcaaccccgtctacccctatggctacgcgcggaatcagaatatccccttcctcactcccccctttgtctcctccgatggattcaaaaacttcccccctggggtactgtcactcaaactggctgatccaatcaccattaccaatggggatgtatccctcaaggtgggaggtggtctcactttgcaagatggaagcctaactgtaaaccctaaggctccactgcaagttaatactgataaaaaacttgagcttgcatatgataatccatttgaaagtagtgctaataaacttagtttaaaagtaggacatggattaaaagtattagatgaaaaaagtgctgcggggttaaaagatttaattggcaaacttgtggttttaacaggaaaaggaataggcactgaaaatttagaaaatacagatggtagcagcagaggaattggtataaatgtaagagcaagagaagggttgacatttgacaatgatggatacttggtagcatggaacccaaagtatgacacgcgcacactttggacaacaccagacacatctccaaactgcacaattgctcaagataaggactctaaactcactttggtacttacaaagtgtggaagtcaaatattagctaatgtgtctttgattgtggtcgcaggaaagtaccacatcataaataataagacaaatccaaaaataaaaagttttactattaaactgctatttaataagaacggagtgcttttagacaactcaaatcttggaaaagcttattggaactttagaagtggaaattccaatgtttcgacagcttatgaaaaagcaattggttttatgcctaatttggtagcgtatccaaaacccagtaattctaaaaaatatgcaagagacatagtttatggaactatatatcttggtggaaaacctgatcagccagcagtcattaaaactacctttaaccaagaaactggatgtgaatactctatcacatttaactttagttggtccaaaacctatgaaaatgttgaatttgaaaccacctcttttaccttctcctatattgcccaagaatga

SEQ ID NO: 37 - protein, 365 aa, Adenovirus serotype 37 fiber
MSKRLRVEDDFNPVYPYGYARNQNIPFLTPPFVSSDGFKNFPPGVLSLKLADPITITNGDVSLKVGGGLTLQDGSLTVNPKAPLQVNTDKKLELAYDNPFESSANKLSLKVGHGLKVLDEKSAAGLKDLIGKLVVLTGKGIGTENLENTDGSSRGIGINVRAREGLTFDNDGYLVAWNPKYDTRTLWTTPDTSPNCTIAQDKDSKLTLVLTKCGSQILANVSLIVVAGKYHIINNKTNPKIKSFTIKLLFNKNGVLLDNSNLGKAYWNFRSGNSNVSTAYEKAIGFMPNLVAYPKPSNSKKYARDIVYGTIYLGGKPDQPAVIKTTFNQETGCEYSITFNFSWSKTYENVEFETTSFTFSYIAQE

SEQ ID NO: 38 - DNA, 1098 bp, Adenovirus serotype 19p fiber
  CDS (1)...(1098)
atgtcaaagaggctccgggtggaagatgacttcaaccccgtctacccctatggctacgcgcggaatcagaatatccccttcctcactcccccctttgtctcctccgatggattcaaaaacttcccccctggggtactgtcactcaaactggctgatccaatcaccattaccaatggggatgtatccctcaaggtgggaggtggtctcactttgcaagatggaagcctaactgtaaaccctaaggctccactgcaagttactactgataaaaaacttgagcttgcatatgataatccatttgaatgtagtgctaataaatttagtttaaaagtaggacatggattaaaagtattagatgaaaaaagtgctgcggggttaaaagatttaattggcaaacttgtggttttaacaggaaaaggaataggcactgaaaatttagaaaatacagatggtagcagcagaggaattggtataaatgtaagagcaagagaagggttgacatttgacaatgatggatacttggtagcatggaacccaaagtatgacacgcgcacactttggacaacaccagacacatctccaaactgcacaattgctcaggataaggactctaaactcactttggtacttacaaagtgtggaagtcaaatattagctaatgtgtctttgattgtggtcgcaggaaagtaccacatcataaataataagacaaatccagaaataaaaagttttactattaaactgttatttaataagaacggagtgcttttagacaactcaaatcttggaaaagcttattggaactttagaagtggaaattccaatgtttcgacagcttatgaaaaagcaattggttttatgcctaatttagtagcgtatccaaaacccagtaattctaaaaaatatgcaagagacatagtttatggaactatatatcttggtggaaaacctgatcagccagcagtcattaaaactacctttaaccaagaaactggatgtgaatactctatcacatttgactttagttggtccaaaacctatgaaaatgttgaatttgaaaccacctcttttaccttctcctatattgcccaagaatga

SEQ ID NO: 39 - protein, 365 aa, Adenovirus serotype 19p fiber
MSKRLRVEDDFNPVYPYGYARNQNIPFLTPPFVSSDGFKNFPPGVLSLKLADPITITNGDVSLKVGGGLTLQDGSLTVNPKAPLQVTTDKKLELAYDNPFECSANKFSLKVGHGLKVLDEKSAAGLKDLIGKLVVLTGKGIGTENLENTDGSSRGIGINVRAREGLTFDNDGYLVAWNPKYDTRTLWTTPDTSPNCTIAQDKDSKLTLVLTKCGSQILANVSLIVVAGKYHIINNKTNPEIKSFTIKLLFNKNGVLLDNSNLGKAYWNFRSGNSNVSTAYEKAIGFMPNLVAYPKPSNSKKYARDIVYGTIYLGGKPDQPAVIKTTFNQETGCEYSITFDFSWSKTYENVEFETTSFTFSYIAQE

SEQ ID NO: 40 - DNA, 1228 bp, Adenovirus serotype 9 fiber
  CDS (50)...(1138)
aagggatgtcaaattcctggtccacaattttcattgtcttccctctcagatgtcaaagaggctccgggtggaagatgacttcaaccccgtctacccctatggctacgcgcggaatcagaatatccccttcctcactcccccctttgtctcctccgatggattccaaaacttcccccctggggtcctgtcactcaaactagctgacccaatagccatcgtcaatgggaatgtctcactcaaagtgggagggggtctcactttgcaagatggaactggaaaactaacagtcaatgctgatccacctttgcaacttacaaacaacaaattagggattgctttggacgctccatttgatgttatagataataaactcacattgttagcgggccatggcttgtctattataacaaaagaaacatcaacactgcctggcttgaggaatactcttgtagtattaactggaaagggtattggaacagaatcaacagataatggcggaacggtatgtgttagagttggagaaggtggcggcttatcatttaataatgatggagacttggtagcatttaataaaaaagaagataagcgcaccctatggacaactccagacacatctccaaattgcaagattgatcaggataaggactctaagttaactctggtccttacaaagtgtggaagtcaaatattggctaatgtgtcattaattgtcgtagatggtaagtacaaaattatcaataacaatactcaaccagctctcaaaggatttaccattaaattattgtttgatgaaaatggagtacttatggaatcttcaaatcttggtaaatcatattggaactttagaaatgaaaattcaattatgtcaacagcttatgaaaaagctattggattcatgcctaatttggtagcctatccaaaacctaccgctggctctaaaaaatatgcaagagatatagtttatggaaacatctaccttggtggaaagccagatcaaccagtaaccattaaaactacctttaatcaggaaactggatgtgaatattctatcacatttgattttagttgggccaagacttatgtaaatgttgaatttgaaacaacctcttttaccttttcctatatcgcccaagaatgaaagaccaataaacgtgtttttcatttcaaaattttcatgtatctttattgatttttacaccagcacgggtagtcagtctcccaccaccag

SEQ ID NO: 41 - protein, 362 aa, Adenovirus serotype 9 fiber
MSKRLRVEDDFNPVYPYGYARNQNIPFLTPPFVSSDGFQNFPPGVLSLKLADPIAIVNGNVSLKVGGGLTLQDGTGKLTVNADPPLQLTNNKLGIALDAPFDVIDNKLTLLAGHGLSIITKETSTLPGLRNTLVVLTGKGIGTESTDNGGTVCVRVGEGGGLSFNNDGDLVAFNKKEDKRTLWTTPDTSPNCKIDQDKDSKLTLVLTKCGSQILANVSLIVVDGKYKIINNNTQPALKGFTIKLLFDENGVLMESSNLGKSYWNFRNENSIMSTAYEKAIGFMPNLVAYPKPTAGSKKYARDIVYGNIYLGGKPDQPVTIKTTFNQETGCEYSITFDFSWAKTYVNVEFETTSFTFSYIAQE

SEQ ID NO: 42 - protein, 20 aa, Artificial Sequence
  Ad2  third repeat
GNLTSQNVTTVTQPLKKTKS

SEQ ID NO: 43 - protein, 20 aa, Artificial Sequence
  Ad5  third repeat
GNLTSQNVTTVSPPLKKTKS

SEQ ID NO: 44 - protein, 4 aa, Artificial Sequence
  Repeat motif
  VARIANT 4: Xaa = Thr or Ser
TTVX

SEQ ID NO: 45 - protein, 15 aa, Artificial Sequence
  Repeat Consensus Sequence
  VARIANT 3,5,7,13: Xaa = Hydrophobic Amino Acid
  VARIANT 1, 2, 4,  6, 8, 9, 11, 12, 14, 15: Xaa = Any Amino Acid
  VARIANT 10: Xaa = Pro or Gly
XXXXXXXXXXXXXXX

SEQ ID NO: 46 - protein, 16 aa, Artificial Sequence
  Ad2 21st repeat
GAMITKLGAGLSFDNS

SEQ ID NO: 47 - protein, 16 aa, Artificial Sequence
  Ad5 21st repeat
KAMVPKLGTGLSFDST

SEQ ID NO: 48 - protein, 16 aa, Artificial Sequence
  Ad37 last repeat
IGINVRAREGLTFDND

SEQ ID NO: 49 - protein, 9 aa, Artificial Sequence
  Last repeat consensus sequence
  VARIANT 4,7: Xaa = Any Amino Acid
  VARIANT 9: Xaa = Asp or Asn
KLGXGLXFX

SEQ ID NO: 50 - DNA, 1164 bp, Artificial Sequence
  Ad5Ds fiber
  CDS (13)...(1092)
  misc_feature 1130, 1157: n = A,T,C or G
atgggatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagaaaaagcttgaagttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaagcggccgcgttatgaagggcgaattccagcacantggcggccgttattagtggatccgagntcatgca

SEQ ID NO: 51 - protein, 359 aa, Artificial Sequence
  Ad5deltas
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 52 - DNA, 1920 bp, Artificial Sequence
  Ad5s/Ad37k fiber
  CDS (13)...(1755)
  misc_feature 1867, 1875: n = A,T,C or G
gcaagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagacactagtccaaactgcacaattgctcaagataaggactctaaactcactttggtacttacaaagtgtggaagtcaaatattagctaatgtgtctttgattgtggtcgcaggaaagtaccacatcataaataataagacaaatccaaaaataaaaagttttactattaaactgctatttaataagaacggagtgcttttagacaactcaaatcttggaaaagcttattggaactttagaagtggaaattccaatgtttcgacagcttatgaaaaagcaattggttttatgcctaatttggtagcgtatccaaaacccagtaattctaaaaaatatgcaagagacatagtttatggaactatatatcttggtggaaaacctgatcagccagcagtcattaaaactacctttaaccaagaaactggatgtgaatactctatcacatttaactttagttggtccaaaacctatgaaaatgttgaatttgaaaccacctcttttaccttctcctatattgcccaagaatgaaaaagcggccgctcgagtctagagggcccgtttaaacccgctgatcagcctcgactgtgccttctagttgccagccatctgttgtttgcccctcccccgtgccttccttgancctggaangtgccactcccactgtcctttcctaataaaatgaggaaatgcatc

SEQ ID NO: 53 - protein, 580 aa, Artificial Sequence
  Ad5s/Ad37k
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPDTSPNCTIAQDKDSKLTLVLTKCGSQILANVSLIVVAGKYHIINNKTNPKIKSFTIKLLFNKNGVLLDNSNLGKAYWNFRSGNSNVSTAYEKAIGFMPNLVAYPKPSNSKKYARDIVYGTIYLGGKPDQPAVIKTTFNQETGCEYSITFNFSWSKTYENVEFETTSFTFSYIAQE

SEQ ID NO: 54 - DNA, 1767 bp, Artificial Sequence
  Ad5s/Ad37s fiber
  CDS (13)...(1749)
atgggatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcagcctaactgtaaaccctaaggctccactgcaagttaatactgattcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacattggtataaatgtaagagcaagagaagggttgacatttgacaatgatggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaagcggccgcgttatg

SEQ ID NO: 55 - protein, 578 aa, Artificial Sequence
  Ad5s/Ad37s
MKRARPSEDTFNPVYPYDTETGPPTVPFLTPPFVSPNGFQESPPGVLSLRLSEPLVTSNGMLALKMGNGLSLDEAGSLTVNPKAPLQVNTDSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNIGINVRAREGLTFDNDGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 56 - DNA, 1132 bp, Artificial Sequence
  Ad37s/Ad5k fiber
  CDS (16)...(1116)
  misc_feature 1125: n = A,T,C or G
gtcgcaagatccaagatgaagagggcccggcccagcgaagatgacttcaaccccgtctacccctatggctacgcgcggaatcagaatatccccttcctcactcccccctttgtctcctccgatggattcaaaaacttcccccctggggtactgtcactcaaactggctgatccaatcaccattaccaatggggatgtatccctcaaggtgggaggtggtctcactttgcaagatggaagcctaactgtaaaccctaaggctccactgcaagttaatactgataaaaaacttgagcttgcatatgataatccatttgaaagtagtgctaataaacttagtttaaaagtaggacatggattaaaagtattagatgaaaaaagtgctgcggggttaaaagatttaattggcaaacttgtggttttaacaggaaaaggaataggcactgaaaatttagaaaatacagatggtagcagcagaggaattggtataaatgtaagagcaagagaagggttgacatttgacaatgatggatacttggtagcatggaacccaaagtatgacacgcgcactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggatccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaagcggncgctcga

SEQ ID NO: 57 - protein, 366 aa, Artificial Sequence
  Ad37s/Ad5k
MKRARPSEDDFNPVYPYGYARNQNIPFLTPPFVSSDGFKNFPPGVLSLKLADPITITNGDVSLKVGGGLTLQDGSLTVNPKAPLQVNTDKKLELAYDNPFESSANKLSLKVGHGLKVLDEKSAAGLKDLIGKLVVLTGKGIGTENLENTDGSSRGIGINVRAREGLTFDNDGYLVAWNPKYDTRTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 58 - protein, 16 aa, Artificial Sequence
  Ad37 third repeat
GSLTVNPKAPLQVNTD

SEQ ID NO: 59 - protein, 14 aa, Artificial Sequence
  Ad8 last repeat
VRVGEGGGLSFNDN

SEQ ID NO: 60 - protein, 14 aa, Artificial Sequence
  Ad9 last repeat
VRVGEGGGLSFNND

SEQ ID NO: 61 - protein, 14 aa, Artificial Sequence
  Ad15 last repeat
VRVGEGGGLSFNEA

SEQ ID NO: 62 - protein, 8 aa, Artificial Sequence
  Penton region
HAIRGDTF

SEQ ID NO: 63 - protein, 15 aa, Artificial Sequence
  Penton amino acid replacement
SRGYPYDVPDYAGTS

SEQ ID NO: 64 - protein, 4 aa, Artificial Sequence
  Fiber protein conserved sequence
TWLT

SEQ ID NO: 65 - protein, 4 aa, Artificial Sequence
  HSP binding motif
KKTK

SEQ ID NO: 66 - protein, 16 aa, Artificial Sequence
  Ad8 third repeat
GKLTVNTEPPLHLTNN

SEQ ID NO: 67 - protein, 16 aa, Artificial Sequence
  Ad9 third repeat
GKLTVNADPPLQLTNN

SEQ ID NO: 68 - protein, 16 aa, Artificial Sequence
  Ad15 third repeat
GNLTVNTEPPLQLTNN

SEQ ID NO: 69 - DNA, 3929 bp, Artificial Sequence
  Vector pCR2.1
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcggcttaagccgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 70 - DNA, 3931 bp, Artificial Sequence
  Vector pCR2.1-Topo
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcgcccttaagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

(19) United States
(12) Patent Application Publication
(10) Pub. No.: US 2007/0003923 A1
(43) Pub. Date: Jan. 4, 2007

(54) Modified fiber proteins for efficient receptor binding

(75) Inventors: Glen R. Nemerow, Encinitas, CA (US); Eugene Wu, Oceanside, CA (US); Phoebe Stewart, Brentwood, TN (US)

Correspondence Address: DLA PIPER RUDNICK GRAY CARY US LLP, 153 TOWNSEND STREET, SUITE 800, SAN FRANCISCO, CA, 94107-1907, US

(73) Assignees: SCRIPPS RESEARCH INSTITUTE, THE, LAJOLLA, CA (US); REGENTS OF THE UNIVERSITY OF CALIFORNIA, THE, OAKLAND, CA (US)

(21) Appl. No.: 10/560,250

(22) PCT Filed: Jun. 10, 2004
(86) PCT No.: PCT/US04/18623

§ 371 Date: Jun. 22, 2006

Related U.S. Application Data
(60) Provisional application No. 60/478,008, filed on Jun. 11, 2003.

(51) Int. Cl. C12Q 1/70; C12N 7/00; C12N 15/861; C07K 14/075

(52) U.S. Cl. 435/5; 435/456; 435/235.1; 530/350; 977/802; 435/325

(57) ABSTRACT

Recombinant detargeted and retargeted adenovirus viral particles and vectors are provided. In particular, modified fibers from adenoviruses that bind to coxsackie-adenovirus receptor (CAR) in vivo that contain modifications in the fiber shaft are provided. Adenovirus (Ad) particles that express such fibers exhibit reduced binding to CAR. Hence detargeted Ad particles are provides; also provided are retargeted particles.

### RELATED APPLICATIONS

Benefit of prioirty is claimed to U.S. provisional application Ser. No. 60/478,008, entitled “MODIFIED FIBER PROTEINS FOR EFFECIEN RECEPTOR BINDING,” filed Jun. 11, 2003, to Glenn Nemerow, Eugene Wu and Phoebe Stewart. Where permitted, the disclosure and subject matter of this application are incorporated by reference in their entirety.

Work described herein was supported by NIH grants EY11431, HL54352 and AI42929. The government has certain rights in such subject matter.

### FIELD OF INVENTION

Recombinant detargeted and retargeted adenovirus viral particles and vectors are provided. In particular, modified fibers from adenoviruses that bind to coxsackie-adenovirus receptor (CAR) in vivo that contain modifications in the fiber shaft are provided. Adenovirus (Ad) particles that express such fibers exhibit reduced binding to CAR. Hence detargeted Ad particles are provided; also provided are retargeted particles.

### BACKGROUND

Most, if not all, adenoviral vector-mediated gene therapy strategies aim to transduce a specific tissue, such as a tumor or an organ. Such targeted delivery requires ablation (detargeting) of a normal virus tropism and typically addition of new specificities (retargeting). Because multiple interactions between adenoviral particles and the host cell are required to promote efficient cell entry (Nemerow (2000) Virology 274:1-4), detargeting and retargeting can be complex. One adenovirus entry pathway is believed to involve two separate cell surface events. First, a high affinity interaction between the adenoviral fiber and a cellular receptor mediates the attachment of the adenovirus particle to the cell surface. A subsequent association of penton protein of the capsid with the cell surface integrins σᵥβ₃ and αᵥβ₅, which act as co-receptors, potentiates virus internalization.

There are a plurality of adenoviral fiber receptors present on various cell types. Fibers of different subgroups of adenoviruses interact with different receptors. One such cell receptor is the coxsackie-adenovirus receptor (CAR), which is expressed in many human tissues including lung epithelial cells (see, e.g., Bergelson et al., (1997) Science 275: 1320-1323). Fibers of all adenoviral subgroups, except subgroup B, have been shown to bind CAR (see, e.g., Bergelson et al., (1997) Science 275: 1320-1323; Roelvink et al., (1998) J. Virol. 72: 589-596). Not all adenoviral subgroups, however, use CAR as their primary cell receptor (Arnberg (2000) J. Virol. 74: 42-48; Roelvink et al., (1996) J. Virol. 70: 7614-7621). Ad37, a subgroup D member, interacts with a 50 kDa protein found on conjunctival cells (Wu et al., (2001) Virology 279: 78-89).

The association between adenoviral fiber and cell surface receptors is a complex, three-dimensional interaction. The recognition between fiber and receptor has been attributed in some cases to specific amino acid residues in the fiber knob, predominantly in the loops between β-strands in the protein structure (Roelvink et al., (1999) Science 286:1568-1571; Bewley et al., (1999) Science 286:1579-1583; Huang et al., (1999) J. Virol. 73:2798-2802). Recognition in vitro and recognition in vivo are not always paralleled. For example, the Ad37 fiber is unable to use CAR efficiently to infect host cells, despite containing a CAR binding site in its knob and binding CAR in in vitro studies (Arnberg (2000) J. Virol. 74: 42-48; Wu et al., (2001) Virology 279: 78-89).

In vivo adenoviral vector retargeting is a major goal in gene therapy and a significant effort has been focused on developing strategies to achieve this goal. For many applications, the most clinically useful adenoviral vector would be deliverable systemically, such as into a peripheral vein, and would be targeted to a desired location in the body, and would not have undesirable side effects resulting from targeting to other locations. Successful targeting strategies therefore would direct the entire vector dose to the appropriate site and would be likely to improve the safety profile of the vector by permitting the use of lower, less toxic vector doses, which also can be potentially less immunogenic. Successful detargeting and retargeting of adenovirus particles has not been achieved.

Thus, there is a need to develop adenoviruses that are detargeted in vivo for use as a base vector and to develop retargeted adenoviruses, such as for specific therapeutic uses. Therefore, among the objects herein, it is an object herein to provide detargeted adenoviral vectors, methods for preparation thereof, and uses thereof. Also among the objects herein, it is an object to provide retargeted adenoviral vectors for therapies, methods of production and uses thereof.

### SUMMARY

Detargeted and retargeted adenoviral particles, adenovirus vectors from which such particles are produced, methods for preparation of the vectors and particles and uses of the vectors and particles are provided. Provided and described are capsid modifications, particularly fiber shaft modifications, and the resulting proteins that, when expressed on adenoviral particles provide for detargeting of adenoviral vectors. The capsid modifications, such as the fiber shaft modifications, can be combined with other modifications, such as fiber knob and/or penton modifications, to produce more fully detargeted and retargeted adenoviral particles. Thus, adenoviral vectors and adenoviral particles whose native tropisms are reduced, including eliminated, through a modification or modifications of capsid proteins, particularly a fiber shaft region, are provided.

Provided are capsid mutations, including fiber shaft modifications, that reduce or modulate binding to particular receptors, particularly Coxsackie-Adenovirus Receptor (CAR), thereby permitting efficient retargeting of adenoviral vectors that contain capsids with such modifications. Provided are adenoviral particles with capsid mutations, including fiber shaft modifications, that reduce binding to particular receptors, thereby permitting efficient retargeting of adenoviral vectors that contain capsids with such modifications.

Particular modifications provided herein are designed to alter the structure of a β-strand or a β-turn in the fiber shaft to thereby alter interaction with CAR. Thus, provided are modified adenovirus (Ad) fiber proteins that include a shaft modification such that binding of the modified fiber to CAR fiber protein shaft is substantially reduced (reduced by at least 50%, 40%, 30%, 10%, 5%, 1% or less) or eliminated (less than 1%, 0.5%, 0.1% or less compared to the unmodified shaft. The modified fibers are from adenovirus particles, such as Ad serotype C, such as Ad2 and Ad5, in which the fiber normally binds to CAR. The fibers include those in which the tertiary structure of modified fiber is altered compared to the structure of the unmodified fiber such that the modified fiber is more rigid than the unmodified fiber.

Modifications include any mutation, such as a deletion, insertion or replacement of at least one amino acid in the fiber shaft, particularly within the repeats of the fiber shaft, such that the resulting fiber exhibits reduced binding to CAR, particularly in vivo. Any amino acids within a repeat can be modified, such as by replacing it with a non-conservative amino acid (see, e.g., TABLE 1 below listing conservative amino acid substitutions) or by eliminating it. Such modifications can be determined empirically by systematically replacing amino acids in a repeat, particularly repeats corresponding to the third and/or last full repeat, and testing the resulting fiber for binding to CAR in vitro. Any fiber that exhibits at least a two-fold, typically a 10, 100 or greater fold reduction in binding in vitro is selected. In the third repeat, the modifications include modifications that include the one or more nucleoties that correspondend to the portion of the third repeat that contains the TTVT/S sequence (SEQ ID No. 44).

The modified fibers also include fibers that have replacements of all or portions of the shaft with a shaft from a fiber that includes repeats that are more rigid than the fiber that binds to CAR, such as fiber shaft, particularly one or more β repeats from a serotype D fiber, such as an Ad37, Ad8, Ad9, Ad15, Ad19p shaft repeat. The replaced repeats in the CAR-binding fiber can be the third repeat and/or last full β repeat in the shaft. The modifications can include deletion of one or more repeats, particularly, deletion of the third repeat and/or last full repeat whereby the resulting fiber does not bind to CAR. Exemplary third repeats from adenoviruses serotype D include those set forth in any of SEQ ID Nos. 58, 66, 67 and 68. Exemplary modified last full repeats from adenovirus serotype D include any of those set forth in any of SEQ ID Nos. 48, 59, 60 and 61. All or portions of each of these repeats can be used to replace the corresponding repeat in fibers of adenoviruses, such as serotype C viruses, that bind to CAR or can serve as templates for modifications of such fibers. Other modifications, include deletion of the one or more of the central repeats, such as 1, 2, 3, 4, 5, 6, 7, 8, 9, 10, 11, 12, 13 or 14 central repeats or other number, depending upon the fiber, that reduces or eliminates CAR binding.

The fiber protein also can include one or more additional modifications in the fiber. Such modifications can further ablate or reduce any binding to CAR and modifications of the fiber shaft that reduce binding to Heparin Sulfate Proteoglycans (also referred to as heparin sulfate glycosaminoglycans; HSP), modifications of the fiber knob, particularly those that further reduce any binding to CAR, and modifications that add ligands to retarget the fiber to other receptors. Detargeted adenoviral particles, adenovirus vectors from which such particles are produced, methods for preparation of the vectors and particles and uses of the vectors and particles are provided. Provided and described are capsid modifications, particularly fiber shaft modifications, and the resulting proteins that, when expressed on adenoviral particles provide for detargeting of adenoviral vectors. The capsid modifications, such as the fiber shaft modifications, can be combined with other modifications, such as fiber knob and/or penton modifications, to produce detargeted adenoviral particles. Particular modifications alter the structure of a β-strand or a β-turn in the fiber shaft. Thus, adenoviral vectors and adenoviral particles whose native tropisms are ablated (reduced or eliminated) through a modification or modifications of capsid proteins, particularly a fiber shaft region, are provided.

Hence, provided are capsid mutations, including fiber shaft modifications, that ablate binding to particular receptors, particularly Coxsackie-Adenovirus Receptor (CAR), thereby permitting efficient detargeting and also retargeting of adenoviral vectors that contain capsids with such modifications.

Provided are modified adenovirus (Ad) fiber proteins that include a shaft modification such that binding of the modified fiber to CAR fiber protein shaft is substantially reduced (reduced by at least 50%, 40%, 30%, 10%, 5%, 1% or more) or eliminated (less than 1%, 0.5%, 0.1%) or less compared to the unmodified shaft. The modified fibers are from adenovirus particles, such as Ad serotype C, such as Ad2 and Ad5, in which the fiber normally binds to CAR. The fibers include those in which the tertiary structure of modified fiber is altered compared to the structure of the unmodified fiber such that the modified is more rigid than the unmodified fiber. Included are modified fibers that are shortened or exhibit reduced flexibility compared to the unmodified fiber.

Modifications include any mutation, such as a deletion, insertion or replacement of at least one amino acid in the fiber shaft, particularly within the repeats of the fiber shaft. The modified fibers can include replacements of all or portions of the shaft with a shaft from a fiber that includes repeats that are more rigid than the fiber that binds to CAR, such as fiber shaft, particularly one or more 1 repeats from a serotype β fiber, such as an Ad37, Ad8, Ad9, Ad15, Ad19p shaft repeat. The replaced repeats in the CAR-binding fiber can be the third and or last full β repeat in the shaft. The modifications can include deletion of one or more repeats, particularly, deletion of the third and/or last full repeat whereby the resulting fiber does not bind to CAR. Exemplary modified third repeats are set forth in those set forth in any of SEQ ID Nos. 58, 66, 67 and 68 and exemplary modified last full repeats includes those set forth in SEQ ID NOS: 48, 59, 60 and 61. Modifications of the third repeat include modifications of nucleotides loci that correspond to TTVT/S in the third β-repeat. Other modifications, include deletion of fourteen central repeats.

The fiber protein also can include one or more additional modifications in the fiber. Such modifications can further ablate or reduce any binding to CAR and/or to Heparin Sulfate Proteoglycans (also referred to as heparin sulfate glycosaminoglycans; HSP). Other modifications of the fiber knob, particularly those that further reduce any binding to CAR, and modifications that add ligands to retarget the fiber to other receptors.

Nucleic acids encoding the fiber proteins, vectors containing the nucleic acids and cells containing the vectors and/or nucleic acids also are provided. Methods using the fibers for detargeting and retargeting of adenoviral particles, particular serotype C particles are provided, as are methods for using the particles for transducing cells, in vivo, in vitro and ex vivo for a variety of applications. Particular embodiments include the following embodiments and embodiments described and exemplified throughout the disclosure herein.

Provided is a modified adenovirus fiber that has a shaft modification in a repeat corresponding to one or both of a third β-repeat or a last full repeat, whereby binding of the fiber or of a viral particle containing such fiber to the coxsackie-adenovirus receptor (CAR) is reduced eliminated compared to the unmodified fiber. The unmodified fibers bind to CAR, and reduction in binding is at least 2-, 5-10-, 100-, 200-, 500-, 1000-fold or more in vivo. In particular, the modified fiber binds to CAR with less than 50%, 40%, 30%, 20%, 10%, 5%, 1% of the binding affinity of the unmodified fiber in vivo and can be assessed by in vitro methods. Typically, the modified fiber is more rigid than the unmodified fiber.

Modifications include, but are not limited to, deletion, insertion or replacement (or other modification) of at least one amino acid in the fiber shaft repeat corresponding to a third repeat and/or a full repeat, including replacement of a third β repeat and/or last full repeat with a corresponding repeat from a serotype D fiber shaft repeat sequence, whereby the resulting fiber exhibits reduced binding to CAR compared to the unmodified fiber. Exemplary modifications include at least one replacement or deletion of one or more amino acids in the fiber in the contiguous sequence of amino acids corresponding to the amino acid sequence set forth in SEQ ID NOS: 42, 43 and/or 44 and SEQ ID NOS: 46 and 47. Generally the unmodified fiber is from a serotype C adenovirus, such as Ad2 or Ad5. Serotype D adenoviruses include, but are not limited to, Ad8, Ad9, Ad15, Ad19p and Ad3. The replacing third repeat can include sequences of amino acids set forth in any of SEQ ID NOS: 58, 66, 67 and 68, and the replacing last full repeat can include a sequence of amino acids, such as any set forth in any of SEQ ID NOS: 48, 59, 60, 61, and SEQ ID No. 49, which represents a consensus sequence. Other repeats can be replaced in addition to or instead of the third and/or last repeats as long as the resulting modified fiber exhibits reduced (at least 2-fold less, typically at least 10-, 50-, 100-fold or more-fold less) binding to CAR.

The modified adenovirus fibers can include one or more than one additional modification in the fiber protein, whereby the modified fiber binds to a receptor other than CAR with greater affinity than the unmodified fiber binds to such receptor or that further reduces binding to CAR or further adds any other desired property. Such modifications include a modification of the Heparin Sulfate Proteoglycans (HSP) binding site in a fiber shaft and modifications of the fiber knob, such as, for example, fiber knobs from an adenovirus that does not interact with CAR. Such adenovirus knobs include those from Ad3 fiber knob, Ad41 short fiber knob, or Ad35 fiber knob. Mutations of the knob include those in the AB loop and/or CD loop, such as KO1 and KO12.

Any of the above modified adenovirus fibers can be from any serotype adenovirus, including a serotype A, B, C or F adenovirus, particularly those that are modified such that at least one amino acid corresponding to the consensus repeat sequence as set forth in SEQ ID No. 45 and/or 49 is modified (deleted, replaced or there is an insertion in the sequence) in the repeat corresponding to either the third repeat or the last full repeat.

Other modified fibers, such as those from serotype C viruses, including Ad2 and Ad5, include those where the unmodified fiber binds the Coxsackie-Adenovirus Receptor (CAR), the fiber protein includes a modification to the fiber protein shaft such that binding of the modified fiber to CAR is substantially reduced or eliminated, the modified fiber shaft contains repeats corresponding to the third repeat and the last full repeat, and at least one repeat of the fiber shaft is deleted. Such other repeats include, for example, repeats 4-17. Deletions include deletion of 5 or more contiguous amino acids corresponding to positions 95-316 of an Ad5 fiber.

Nucleic acid molecules encoding the modified fibers are provided. Included among the nucleic acid molecules are vectors, particularly, for example, adenovirus vectors, which also can include heterologous nucleic acid. The heterologous nucleic acid, for example, can be a regulatory sequence or can encode a gene product, such as, but are not limited to, therapeutic products. Adenovirus vectors, include, but are not limited to, early generation adenoviral vectors, gutless adenoviral vectors and replication-conditional adenoviral vectors, such as, for example, oncolytic vectors. Cells, including eukaryotic and prokaryotic cells, that contain the nucleic acid molecules also are provided. Included among the cells are cells from packaging cell lines. Also provided are packaging cell lines that contain the cells, particularly, the cells that contain nucleic acid that encodes the modified fiber as a separate construct from the adenoviral genome.

Adenoviral particles that express the modified fibers also are provided. The particles can further include additional capsid modifications, such as, but are not limited to, a penton modification. The particles can be such that the N-terminal portion of the fiber is from the same serotype as the genome so that incorporation of the fiber into the capsid is facilitated. Typically, the N-terminal portion of the modified fiber includes at least the N-terminal 15, 16 or 17 amino acids of such fiber. The particles also can include a targeting ligand in the capsid, such as in the fiber, for retargeting or targeting of the particles to selected cells or tissues. The particles can include further modifications of the capsid, including the fiber to alter additional binding or targeting properties of the particle. For example, the particles can include a modified fiber such that binding to HSP is altered compared to a particle that expresses an unmodified capsid, and/or can include a mutation in the aV integrin-binding region of the capsid, whereby binding to the integrin is eliminated or reduced, and/or further modifications, such as knob modification, such as a modification in the AB and/or CD loop, to further reduce or eliminate any CAR binding.

Also provided are methods of detargeting an adenoviral vector by reducing or eliminating the binding of an adenoviral particle to CAR by producing an adenoviral particle that expresses any of the modified fibers.

Compositions formulated for administration to a subject are provided. The compositions contain the adenovirus particles.

Methods for treatment by administering the compositions are provided. The compositions can be administered in vivo or ex vivo by introducing the adenoviral particles into cells or into the subject for trafficking to selected target cells.

### BRIEF DESCRIPTION OF THE FIGURES

FIGS. 1A and 1B set forth exemplary repeat alignments of the third repeat and the last full repeat sequences from adenovirus fiber proteins of Ad2, Ad5, Ad37, Ad8, Ad9 and Ad15; a consensus sequence for the last full repeat is set forth in FIG. 1B (see, SEQ ID No. 49).

FIG. 2 presents a schematic of fiber chimeras and the length and flexibility properties of each; Ad5 regions are shown in light gray and Ad37 regions are shown in black; repeats that contribute to the flexibility of the fiber are shown as striped ovals; pluses and minuses indicate the relative length or flexibility of the fiber or fiber chimera.

### DETAILED DESCRIPTION

A. DEFINITIONS

B. Capsid modifications
1. Fiber genes and proteins
2. The fiber shaft
3. Modifications of the fiber shaft

C. Nucleic acids, Adenoviral vectors and cells containing the nucleic acids and cells containing the vectors
1. Adenoviral vectors and particles
   a. Gutless vectors
   b. Oncolytic vectors
   c. Helper independent viruses
2. Packaging and complementing cell lines

D. Detargeting

E. Retargeting
1. Addition of targeting ligand
2. Retargeting achieved through modified fibers

F. Delivery of heterologous products
1. Heterologous Polypeptides
2. Gene Expression and Regulation
   a. Heterologous polynucleotides
   b. Regulation of gene expression

G. Animal and human delivery

H. Formulation and administration

A. DEFINITIONS

Unless defined otherwise, all technical and scientific terms used herein have the same meaning as is commonly understood by one of skill in the art of which the invention(s) belong. All patents, patent applications, published applications and publications, GENBANK sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, the term “adenovirus” or “adenoviral particle” is used to include any and all viruses that can be categorized as an adenovirus, including any adenovirus that infects a human or an animal, including all groups, subgroups, and serotypes, and refers to particles that include encapsulated nucleic acid. The nucleic acid can be a native adenoviral genome or a modified genome, such as an adenovirus vector. There are at least 51 serotypes of Adenovirus that are classified into several subgroups. For example, subgroup A includes adenovirus serotypes 12, 18, and 31. Subgroup C includes adenovirus serotypes 1, 2, 5, and 6. Subgroup D includes adenovirus serotype 8, 9, 10, 13, 15, 17, 19, 20, 22-30, 32, 33, 36-39 and 42-49. Serotype 19 has variants “a” and “p”. Ad19p is a nonpathogenic variant of Ad19 (Arnberg et al. (1998) Virology 227:239-244) while Ad19a, along with Ad8 and Ad37, are major causes of epidemic keratoconjunctivitis (EKC). Ad19a and Ad37 have identical fiber proteins (Arnberg et al. (1998) Virology 227:239-244) and have similar tropism in vivo. Subgroup E includes adenovirus serotype 4. Subgroup F includes adenovirus serotypes 40 and 41. These latter two serotypes have a long and a short fiber protein. Thus, as used herein, an adenovirus or adenovirus particle is a packaged vector or genome.

As used herein, “virus,” “viral particle,” “vector particle,” “viral vector particle,” and “virion” are used interchangeably to refer to infectious viral particles that are formed when, for example, a vector containing all or a part of a viral genome, is transduced into an appropriate cell or cell line for the generation of such particles. The resulting viral particles have a variety of uses, including, but not limited to, transferring nucleic acids into cells either in vitro or in vivo. For purposes herein, the viruses are adenoviruses, including recombinant adenoviruses formed when an adenovirus vector, such as any provided herein, is encapsulated in an adenovirus capsid. Thus, a viral particle is a packaged viral genome. An adenovirus viral particle is the minimal structural or functional unit of a virus. A virus can refer to a single particle, a stock of particles or a viral genome. The adenovirus (Ad) particle is relatively complex and can be resolved into various substructures.

Included among adenoviruses and adenoviral particles are any and all viruses that can be categorized as an adenovirus, including any adenovirus that infects a human or an animal, including all groups, subgroups, and serotypes. Thus, as used herein, “adenovirus” and “adenovirus particle” refer to the virus itself and derivatives thereof, and cover all serotypes and subtypes and naturally occurring and recombinant forms, except where indicated otherwise. Adenovirus and adenoviral can be abbreviated as “Ad”. Included are adenoviruses that infect human cells. Adenoviruses can be wildtype or can be modified in various ways known in the art or as disclosed herein. Such modifications include, but are not limited to, modifications of the adenovirus genome that is packaged in the particle in order to make an infectious virus. Exemplary modifications include deletions known in the art, such as deletions in one or more of the E1a, E1b, E2a, E2b, E3, or E4 coding regions. Other exemplary modifications include deletions of all of the coding regions of the adenoviral genome. Such adenoviruses are known as “gutless” adenoviruses. The terms also include replication-conditional adenoviruses, which are viruses that preferentially replicate in certain types of cells or tissues but to a lesser degree or not at all in other types. For example, among the adenoviral particles provided herein, are adenoviral particles that replicate in abnormally proliferating tissue, such as solid tumors and other neoplasms. These include the viruses disclosed in U.S. Pat. No. 5,998,205 and U.S. Pat. No. 5,801,029. Such viruses are sometimes referred to as “cytolytic” or “cytopathic” viruses (or vectors), and, if they have such an effect on neoplastic cells, are referred to as “oncolytic” viruses (or vectors). As used herein, oncolytic adenoviruses refer to adenoviruses that replicate selectively in tumor cells.

As used herein, the terms “vector,” “polynucleotide vector,” “polynucleotide vector construct,” “nucleic acid vector construct,” and “vector construct” are used interchangeably herein to mean any nucleic acid construct that can be used for gene transfer, as understood by those skilled in the art.

As used herein, the term “viral vector” is used according to its art-recognized meaning. It refers to a nucleic acid vector construct that includes at least one element of viral origin and can be packaged into a viral vector particle. The viral vector particles can be used for the purpose of transferring DNA, RNA or other nucleic acids into cells either in vitro or in vivo. Viral vectors include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, Sindbis vectors, semliki forest virus vectors, phage vectors, adenoviral vectors, and adeno-associated viral (AAV) vectors. Suitable viral vectors are described, for example, in U.S. Pat. Nos. 6,057,155, 5,543,328 and 5,756,086. The vectors provided herein are adenoviral vectors.

As used herein, “adenovirus vector”, “adenoviral vector” are used interchangeably and are well understood in the art to mean a polynucleotide containing all or a portion of an adenovirus genome. An adenoviral vector refers to nucleic encoding a complete genome or a modified genome, or one that can be used to introduce heterologous nucleic acid when transferred into a cell, particularly when packaged as a particle. An adenoviral vector can be in any of several forms, including, but not limited to, naked DNA, DNA encapsulated in an adenovirus capsid, DNA packaged in another viral or viral-like form (such as herpes simplex, and AAV), DNA encapsulated in liposomes, DNA complexed with polylysine, DNA complexed with synthetic polycationic molecules, DNA conjugated with transferrin, DNA complexed with compounds such as PEG to immunologically “mask” the molecule and/or increase half-life, or DNA conjugated to a non-viral protein.

As used herein, a variety of vectors with different requirements and purposes are described. For example, one vector is used to deliver particular nucleic acid molecules into a packaging cell line for stable integration into a chromosome. These types of vectors also are referred to as complementing plasmids. A further type of vector carries or delivers nucleic acid molecules in or into a cell line (e.g., a packaging cell line) for the purpose of propagating viral vectors; hence, these vectors also can be referred to herein as delivery plasmids. A third “type” of vector is the vector that is in the form of a virus particle encapsulating a viral nucleic acid and that contains a capsid modified as provided herein. Such vectors also can contain heterologous nucleic acid molecules encoding particular polypeptides, such as therapeutic polypeptides or regulatory proteins or regulatory sequences to target specific cells or cell types in a subject in need of treatment.

As used herein, the term “motif” is used to refer to any set of amino acids forming part of a primary sequence of a protein, either contiguous or capable of being aligned to certain invariant or conserved positions, that is associated with a particular function. The motif can occur, not only by virtue of the primary sequence, but also as a consequence of three-dimensional folding. For example, the motif GXGXXG is associated with nucleotide-binding sites. In another example, the fiber is a trimer, hence the trimeric structure can contribute to the formation of a motif. Alternatively, a motif can be considered as a domain of a protein, where domain is a region of a protein molecule delimited on the basis of function without knowledge of and relation to the molecular substructure, as, e.g., the part of a protein molecule that binds to a receptor. For example, the motif KKTK (SEQ ID No. 65) constitutes a consensus sequence for fiber shaft interaction with HSP (Heparin Sulfate Proteoglycans; also referred to as heparin sulfate glycosaminoglycans).

As used herein, the term “bind” or “binding” is used to refer to the binding between a ligand and its receptor, such as the binding of an Ad5 shaft motif with HSP (Heparin Sulfate Proteoglycans), with a K_{d} in the range of 10⁻² to 10⁻¹⁵ mole/l, generally, 10⁻⁶ to 10⁻¹⁵, 10⁻⁷ to 10⁻¹⁵ and typically 10⁻⁸ to 10⁻¹⁵ (and/or a Kₐ of 10⁵-10¹², 10⁷-10¹², 10⁸-10¹² l/mole).

As used herein, specific binding or selective binding means that the binding of a particular ligand and one receptor interaction (kₐ or K_{eq}) is at least 2-fold, generally, 5, 10, 50, 100 or more-fold, greater than for another receptor. A statement that a particular viral vector is targeted to a cell or tissue means that its affinity for such cell or tissue in a host or in vitro is at least about 2-fold, generally, 5, 10, 50, 100 or more-fold, greater than for other cells and tissues in the host or under the in vitro conditions.

As used herein, the term “ablate” or “ablated” is used to refer to an adenovirus, adenoviral vector or adenoviral particle, in which the ability to bind to a particular cellular receptor is reduced or eliminated, generally substantially eliminated (i.e., reduced more than 10-fold, 100-fold or more) when compared to a corresponding wild-type adenovirus. An ablated adenovirus, adenoviral vector or adenoviral particle also is said to be detargeted, i.e., when the modified adenovirus, adenoviral vector or adenoviral particle does not possess the native tropism of the wild-type adenovirus. The reduction or elimination of the ability of the mutated adenovirus fiber protein and/or mutated adenovirus penton protein to bind a cellular receptor as compared to the corresponding wild-type fiber protein and/or wild-type penton protein can be measured or assessed by comparing the transduction efficiency (gene transfer and expression of a marker gene) of an adenovirus particle containing the mutated fiber protein and/or mutated penton protein compared to an adenovirus particle containing the wild-type fiber protein and/or wild-type penton protein into cells having the cellular receptor.

As used herein, binding of the modified fiber to CAR fiber protein shaft is said by reduced when it is reduced by at least 50%, 40%, 30%, 10%, 5%, 1% or more and is said to be eliminated when it is less than 1%, 0.5%, 0.1% or less compared to the unmodified shaft in vivo. Binding is initially assessed in in vitro assays. For the particular modifications provided herein, observation of reduction of binding to CAR in vitro correlates with a reduction in vivo.

As used herein, tropism with reference to an adenovirus, refers to the selective infectivity or binding that is conferred on the particle by a capsid protein, such as the fiber protein and/or penton.

As used herein, “penton” or “penton complex” is used herein to designate a complex of penton base and fiber. The term “penton” also is used to indicate penton base, as well as penton complex. The meaning of the term “penton” alone should be clear from the context within which it is used.

As used herein, the term “substantially eliminated” with respect to transduction efficiency refers to a transduction efficiency of less than about 50%, typically less than about 11%, of the efficiency of the wild-type fiber containing virus. Generally, the reduced transduction efficiency is less than about 9%, and typically less than about 8% of the wild-type fiber containing virus. The transduction efficiency on cells can be measured by any method known to those of skill in the art (see, e.g., Example 1 of U.S. application Ser. No. 09/870,203 filed on 30 May 2001, and published as U.S. Published application No. 20020137213, and of International Patent Application No. PCT/EP01/06286 filed 1 Jun. 2001). Briefly, cells are infected with the adenoviral particles that contain mutated fiber proteins to evaluate the effects of fiber amino acid mutations on CAR interaction and subsequent gene expression. Monolayers of cells in 12-well dishes are infected with, for example, 1000 particles per cell for 2 hours at 37° C. in a total volume of, for example, 0.35 ml of the DMEM containing 2% FBS. The infection medium is then aspirated from the monolayers and 1 ml of complete DMEM containing 10% FBS was added per well. The cells are incubated for a sufficient time, generally about 24 hours, to allow for β-galactosidase expression, which is measured by a chemiluminescence reporter assay and by histochemical staining with a chromogenic substrate. The relative levels of β-galactosidase activity are determined using a suitable system, such as the Galacto-Light chemiluminescence reporter assay system (Tropix, Bedford, Mass.) Cell monolayers are washed with PBS and processed according to the manufacturer's protocol. The cell homogenate is transferred to a microfuge tube and centrifuged to remove cellular debris. Total protein concentration is determined, such as by using the bicinchoninic acid (BCA) protein assay (Pierce, Inc., Rockford, Ill.) with bovine serum albumin as the assay standard. An aliquot of each sample is then incubated with the Tropix β-galactosidase substrate for 45 minutes in a 96-well plate. A luminometer is used to determine the relative light units (RLU) emitted per sample and then normalized for the amount of total protein in each sample (RLU/ug total protein). For the histochemical staining procedure, the cell monolayers are fixed with 0.5% glutaraldehyde in PBS, and then are incubated with a mixture of 1 mg of 5-bromo-4-chloro-3-indolyl-β-D-galactoside (X-gal) per ml, 5 mM potassium ferrocyanide, 5 mM potassium ferricyanide and 2 mM MgCl₂ in 0.5 ml of PBS. The monolayers are washed with PBS and the blue cells are visualized by light microscopy, such as with a Zeiss IDO3 microscope.

As used herein, the phrase “reduce” or “reduction” refers to a change in the efficiency of transduction by an adenovirus containing a mutated fiber compared to the same adenovirus except that it contains the wild-type fiber. Typically the reduction is about 90%, 80%, 75% or less than the wild-type. Generally, the change in efficiency is to a level of about 65% or less than wild-type. Typically it is about 55% or less. This system of transduction efficiency comparisons is able to rapidly analyze modified fiber proteins and/or modified penton proteins for desired tropism in the context of the viral particle.

As used herein, the terms “mutate”, “mutation”, “modify” and “modification” refer to the deletion, insertion, replacement or change of at least one amino acid in the protein of interest. The amino acid can be changed by substitution or by modification in a way that derivatizes the amino acid. Thus, for example, at least one amino acid of the sequence KLGXGLXFD/N (SEQ ID No. 49), where X can be any amino acid, in an adenovirus fiber is mutated to ablate the viral interaction with CAR.

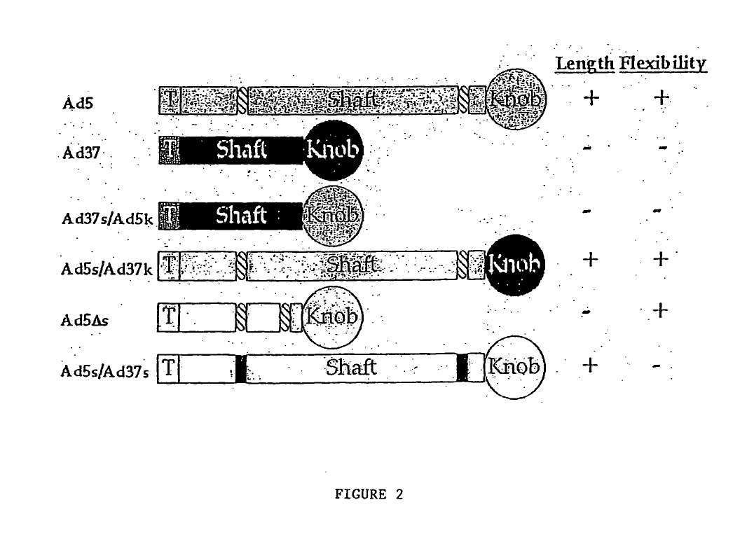

As used herein, the term “chimeric” such as in the context of “chimeric protein” or “chimeric fiber” refers to a protein or polypeptide in which at least a portion, typically a portion containing more than 5 or 6 contiguous amino acids, of the protein are different from the wild-type protein. Chimeric proteins can be fusions of a wildtype protein with a second protein or portion thereof or a peptide. Chimeric proteins include proteins that have one region of the protein replaced with the region from another protein. For example, as described herein, chimeric fibers are constructed with the knob region from one adenovirus fiber joined to the tail and shaft regions from another Ad fiber. Also described herein are chimeric fibers that contain shaft regions made up of repeats from different Ad fibers. Examples of chimeric fiber proteins are shown in FIG. 2.

As used herein, the term “repeat” means a sequence of amino acids that occurs more than once within a polypeptide. In some cases, the repeats will be identical in amino acid sequence to one another. In other cases, the repeats are not identical; they can resemble a consensus sequence derived from comparison of some or all of the repeats within a protein or proteins. For example, as described herein, the adenovirus fiber shaft has repeats of amino acids, approximately 15 amino acids in length. The number of repeats within each fiber shaft varies between adenovirus fibers. Each of these repeats resembles a consensus sequence abCdEfGhijKlMno (see, SEQ ID No. 45) where capitalized letters represent hydrophobic amino acids and the underlined residue (j) denotes the special proline or glycine that allows the β strands to form a β-turn.

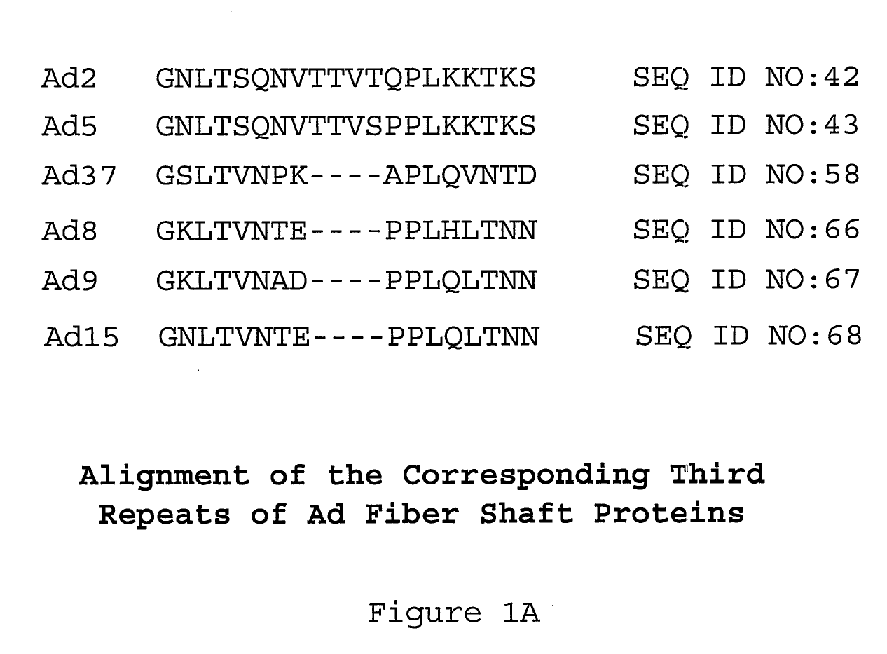

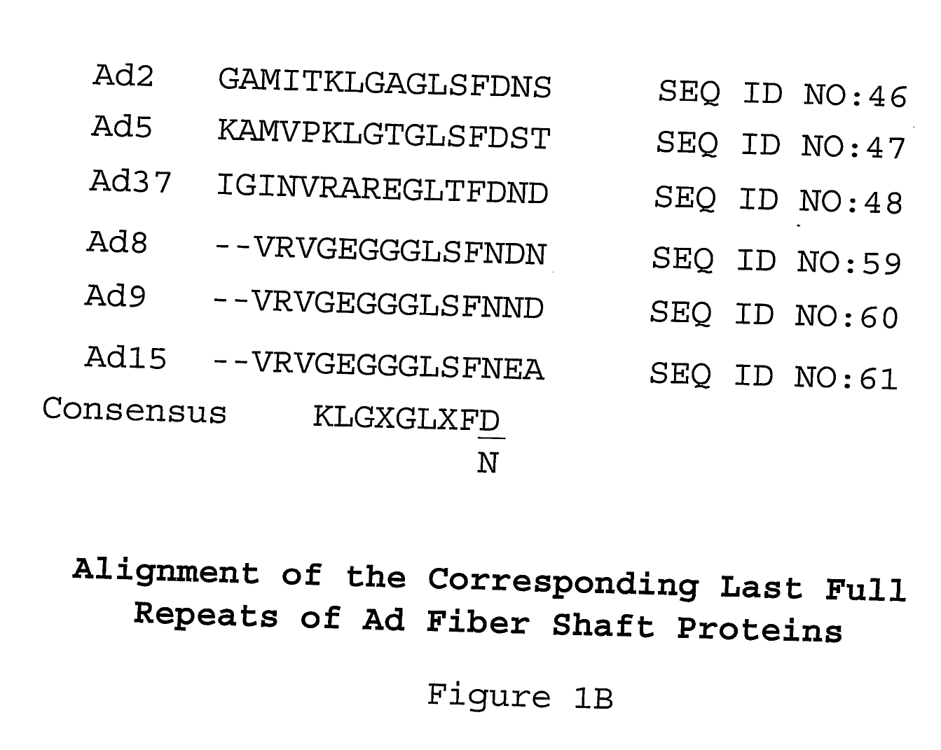

As used herein, “at a position corresponding to” refers to a position (i.e., base number or residue number) in a nucleic acid molecule or protein relative to the position in another reference nucleic acid molecule or protein. Corresponding positions can be determined by comparing and aligning sequences to maximize the number of matching nucleotides or amino acid residues, for example, such that similarity between the sequences is greater than 25%, typically greater than 40%. The position of interest is then given the number assigned in the reference nucleic acid molecule. For example, it is shown herein that the third repeat in the fiber shaft of Ad5 occurs at amino acids 76-95 of SEQ ID No. 35. To identify the corresponding repeat in another adenovirus fiber, the sequences are aligned and then the positions that line up with amino acids 76-95 are determined. Since different adenovirus fibers can be of different lengths, the position designated amino acid 76 may not be amino acid 76, but instead is at a position that “corresponds” to the position in the reference sequence. Similarly, the repeat designated the third repeat in Ad5 may not be the third repeat of a different adenovirus fiber, but at a position such that the amino acids “correspond” to the amino acids of the third repeat. Exemplary repeats corresponding to the third repeat and to the last full repeat in fiber proteins from different adenoviruses are shown in FIGS. 1A and 1B.

As used herein, the term “polynucleotide” means a nucleic acid molecule, such as DNA or RNA. The molecule can include regulatory sequences, and is generally DNA. Such polynucleotides are prepared or obtained by techniques known by those skilled in the art in combination with the teachings contained therein.

As used herein, the terms “protein” and “polypeptide” are used interchangeably.

As used herein, “homologous” means about greater than 25% nucleic acid sequence identity, such as 25% 40%, 60%, 70%, 80%, 90% or 95%. If necessary the percentage homology will be specified. The terms “homology” and “identity” are often used interchangeably. In general, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence identity, the number of conserved amino acids are determined by standard alignment algorithms programs, and are used with default gap penalties established by each supplier. Substantially homologous nucleic acid molecules would hybridize typically at moderate stringency or at high stringency all along the length of the nucleic acid or along at least about 70%, 80% or 90% of the full-length nucleic acid molecule of interest. Also contemplated are nucleic acid molecules that contain degenerate codons in place of codons in the hybridizing nucleic acid molecule.

Whether any two nucleic acid molecules have nucleotide sequences that are at least, for example, 80%, 85%, 90%, 95%, 96%, 97%, 98% or 99% “identical” can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar® “MegAlign” program (Madison; Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids) that are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps. Therefore, as used herein, the term “identity” represents a comparison between a test and a reference polypeptide or polynucleotide.

As used herein, the term “alignment” represents a comparison between a test and a reference polypeptide or portion thereof such as the comparison of a repeat or repeats from an Ad5 fiber shaft to regions of a fiber shaft from another adenovirus fiber shaft such as Ad37 repeats. This alignment determines the corresponding positions or corresponding repeats as defined herein.

As used herein, the term “at least 90% identical to” refers to percent identities from 90 to 100% relative to the reference polypeptide or polynucleotide. Identity at a level of 90% or more is indicative of the fact that, assuming for exemplification purposes a test and reference polypeptide length of 100 amino acids are compared, no more than 10% (i.e., 10 out of 100) of amino acids in the test polypeptide differs from that of the reference polypeptides. Similar comparisons can be made between test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, or deletions. At the level of homologies or identities above about 85-90%, the result should be independent of the program and gap parameters set; such high levels of identity can be assessed readily, often without relying on software.

As used herein: stringency of hybridization in determining percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

Those of skill in this art know that the washing step selects for stable hybrids (see, e.g., Sambrook, E. F. Fritsch, T. Maniatis, in: Molecular Cloning, A Laboratory Manual, Cold Spring Harbor Laboratory Press (1989), vol. 3, p. B.13, see, also, numerous catalogs that describe commonly used laboratory solutions). SSPE is pH 7.4 phosphate-buffered 0.18 M NaCl. Further, those of skill in the art recognize that the stability of hybrids is determined by Tₘ, which is a function of the sodium ion concentration and temperature (Tₘ=81.5° C.−16.6(log₁₀[Na⁺])+0.41 (% G+C)−600/l)), so that the only parameters in the wash conditions critical to hybrid stability are sodium ion concentration in the SSPE (or SSC) and temperature.

It is understood that equivalent stringencies can be achieved using alternative buffers, salts and temperatures. By way of example and not limitation, procedures using conditions of low stringency are as follows (see also Shilo and Weinberg, Proc. Natl. Acad. Sci. USA 78:6789-6792 (1981)): Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA (10×SSC is 1.5 M sodium chloride, and 0.15 M sodium citrate, adjusted to a pH of 7).

Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and re-exposed to film. Other conditions of low stringency that can be used are well known in the art (e.g., as employed for cross-species hybridizations).

By way of example and not way of limitation, procedures using conditions of moderate stringency include, for example, but are not limited to, procedures using such conditions as follows: Filters containing DNA are pretreated for 6 hours at 55° C. in a solution containing 6×SSC, 5× Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 18-20 hours at 55° C. Washing of filters is done at 37° C. for 1 hour in a solution containing 2×SSC, 0.1% SDS and then by washing twice for 30 minutes at 60° C. in a solution containing 1×SSC and 0.1% SDS. Filters are blotted dry and exposed for autoradiography. Other conditions of moderate stringency that can be used are well-known in the art.

By way of example and not way of limitation, procedures using conditions of high stringency are as follows: Prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 hours at 65° C. in prehybridization mixture containing 100 μg/ml denatured salmon sperm DNA and 5-20×10⁶ cpm of ³²P-labeled probe. Washing of filters is done at 37° C. for 1 hour in a solution containing 2×SSC, 0.01% PVP, 0.01% Ficoll, and 0.01% BSA. This is followed by a wash in 0.1×SSC at 50° C. for 45 minutes before autoradiography. Other conditions of high stringency that can be used are well known in the art.

The terms substantially identical, or substantially homologous or similar vary with the context as understood by those skilled in the relevant art and generally means at least 60% or 70%, preferably means at least 80%, 85% or more preferably at least 90%, and most preferably at least 95% identity.

For purposes herein, amino acid substitutions can be made by making conservative amino acid substitutions and also non-conservative amino acid substitutions and then, if necessary testing the resulting fiber for CAR binding activity in vitro. Amino acid substitutions for eliminating activity (i.e., CAR binding) typically are made using non-conservative amino acids. Conservative substitutions of amino acids are known to those of skill in this art and can be made generally without altering the biological activity, for example enzymatic activity, of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). The substitutions contemplated herein are in essential regions, the β-repeats in the fiber shaft. Hence, substitution of any amino acid for another can reduce CAR binding activity. Conservative amino acid substitutions are made, for example, in accordance with those set forth in TABLE 1 as follows:

TABLE 1

| Original residue | Conservative substitution |
|---|---|
| Ala (A) | Gly; Ser, Abu |
| Arg (R) | Lys, orn |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val; Met; Nle; Nva |
| Leu (L) | Ile; Val; Met; Nle; Nv |
| Lys (K) | Arg; Gln; Glu |
| Met (M) | Leu; Tyr; Ile; Nle Val |
| Ornithine | Lys; Arg |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu; Met; Nle; Nv |

Substitutions can be determined empirically or in accordance with known properties and/or can be determined in silico.

As used herein, in silico refers to research and experiments performed using a computer. In silico methods include, but are not limited to, molecular modelling studies, biomolecular docking experiments, and virtual representations of molecular structures and/or processes, such as molecular interactions.

As used herein, adenoviral genome is intended to include any adenoviral vector or any nucleic acid molecule, including any Ad vector or nucleic acid comprising a modified fiber protein. All adenovirus serotypes are contemplated for use in the vectors and methods herein.

As used herein, a packaging cell line is a cell line that is able to package adenoviral genomes or modified genomes to produce viral particles. It can provide a missing gene product or its equivalent. Thus, packaging cells can provide complementing functions for the genes deleted in an adenoviral genome (e.g., the nucleic acids encoding modified fiber proteins) and are able to package the adenoviral genomes into the adenovirus particle. The production of such particles requires that the genome be replicated and that those proteins necessary for assembling an infectious virus are produced. The particles also can require certain proteins necessary for the maturation of the viral particle. Such proteins can be provided by the vector or by the packaging cell.

As used herein, detargeted adenoviral particles have ablated (reduced or eliminated) interaction with receptors with which native particles interact. Detargeted particles have two or more specificities altered. It is understood that in vivo no particles are ablated such that they do not interact with any cells. Detargeted particles have reduced, typically substantially reduced, or eliminated interaction with native receptors. For purposes herein, detargeted particles have reduced (2-fold, 5-fold, 10-fold, 100-fold or more) binding or virtually no binding to CAR; detargeted vector particles include further capsid modifications to eliminate interactions with other cell receptors, HSP and integrins. The particles still bind to cells, but the types of cells and interactions are reduced.

As used herein, pseudotyping describes the production of adenoviral vector particles with modified capsid protein or capsid proteins from a serotype different from the serotype of the vector itself. One example, is the production of an adenovirus 5 vector particle containing an Ad37 or Ad35 fiber protein. This can be accomplished, for example, by producing the adenoviral vector particle in packaging cell lines expressing different fiber proteins. As provided herein, detargeting of an adenovirus 5 particle or other serotype group C adenovirus or other adenovirus that binds to CAR to reduce or eliminate binding to CAR can be effected by replacing, mutating or deleting at least one of the repeat sequences within the fiber shaft.

As used herein, receptor refers to a biologically active molecule that specifically or selectively binds to (or with) other molecules. The term “receptor protein” can be used to more specifically indicate the proteinaceous nature of a specific receptor.

As used herein, “interact” and “interaction” such as in the context of fiber-receptor interactions refer to associations between molecules. These can involve direct and/or indirect associations, binding, and/or recognition between the molecules.

As used herein, the term “cyclic RGD” (or cRGD) refers to any amino acid that binds to αᵥ integrins on the surface of cells and contains the sequence RGD (Arg-Gly-Asp).

As used herein, the term “heterologous polynucleotide” means a polynucleotide derived from a biological source other than an adenovirus or from an adenovirus of a different serotype or it can be a polynucleotide that is in a different locus from wild-type virus. The heterologous polynucleotide can encode a polypeptide, such as a toxin or a therapeutic protein. The heterologous polynucleotide can contain regulatory regions, such as a promoter region, such as a promoter active in specific cells or tissue, for example, tumor tissue as found in oncolytic adenoviruses. Alternatively, the heterologous polynucleotide can encode a polypeptide and further contain a promoter region operably linked to a coding region.

As used herein, reference to an amino acid in an adenovirus protein or to a nucleotide in an adenovirus genome is with reference to Ad5, unless specified otherwise. Corresponding amino acids and nucleotides in other adenovirus strains and modified strains and in vectors can be identified by those of skill in the art. Thus recitation of a mutation is intended to encompass all adenovirus strains that possess a corresponding locus.

As used herein, the KO mutations refer to mutations in fiber proteins that knock out binding to CAR such as those exemplified in U.S. application Ser. Nos. 10/351,890 and 60/459,000. For example, a KO1 mutation refers to a mutation in the Ad5 fiber and corresponding mutations in other fiber proteins. In Ad5, this mutation results in a substitution of fiber amino acids 408 and 409, changing them from serine and proline to glutamic acid and alanine, respectively. As used herein, a KO12 mutation refers to a mutation in the Ad5 fiber and corresponding mutations in other fiber proteins. In Ad5, this mutation is a four amino acid substitution in SEQ ID No. 35 as follows: R512S, A515G, E516G, and K517G. Other KO mutations can be identified empirically or are known to those of skill in the art.

As used herein, PD mutations refer to mutations in the penton gene that ablate binding to αᵥ integrin by replacing the RGD tripeptide. For example, the PD1 mutation exemplified in U.S, Application No. 60/459,000 results in a substitution of amino acids 337 through 344 of the Ad5 penton protein, HAIRGDTF (SEQ ID No. 61), with amino acids SRGYPYDVPDYAGTS (SEQ ID No. 62), thereby replacing the RGD tripeptide.

As used herein, treatment means any manner in which the symptoms of a condition, disorder or disease are ameliorated or otherwise beneficially altered.

As used herein, a therapeutically effective product is a product that is encoded by heterologous DNA that, upon introduction of the DNA into a host, a product is expressed that effectively ameliorates or eliminates the symptoms, manifestations of an inherited or acquired disease or that cures said disease.

As used herein, a subject is an animal, such as a mammal, typically a human, including patients.

As used herein, genetic therapy involves the transfer of heterologous DNA to certain cells, target cells, of a mammal, particularly a human, with a disorder or conditions for which such therapy is sought. The DNA is introduced into the selected target cells in a manner such that the heterologous DNA is expressed and a therapeutic product encoded thereby is produced. Alternatively, the heterologous DNA can in some manner mediate expression of DNA that encodes the therapeutic product, it can encode a product, such as a peptide or RNA that in some manner mediates, directly or indirectly, expression of a therapeutic product. Genetic therapy also can be used to deliver nucleic acid encoding a gene product to replace a defective gene or supplement a gene product produced by the mammal or the cell in which it is introduced. The introduced nucleic acid can encode a therapeutic compound, such as a growth factor or inhibitor thereof, or a tumor necrosis factor or inhibitor thereof, or a receptor therefor, that is not normally produced in the mammalian host or that is not normally produced in therapeutically effective amounts or at a therapeutically useful time. The heterologous DNA encoding the therapeutic product can be modified prior to introduction into the cells of the afflicted host in order to enhance or otherwise alter the product or expression thereof.

As used herein, a therapeutic nucleic acid is a nucleic acid that encodes a therapeutic product. The product can be nucleic acid, such as a regulatory sequence or gene, or can be a protein that has a therapeutic activity or effect. For example, therapeutic nucleic acid can be a ribozyme, antisense, double-stranded RNA, a nucleic acid encoding a protein and others.

As used herein, substantially identical to a product means sufficiently similar so that the property of interest is sufficiently unchanged so that the substantially identical product can be used in place of the product.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

The methods and preparation of products provided herein, unless otherwise indicated, employ conventional techniques of chemistry, molecular biology, microbiology, recombinant DNA, genetics, immunology, cell biology, cell culture and transgenic biology, which are within the skill of the art (see, e.g., Maniatis et al. (1982) Molecular Cloning: A Laboratory Manual, Cold Spring Harbor Laboratory Press, Cold Spring Harbor, N.Y.); Sambrook et al. (1989) Molecular Cloning: A Laboratory Manual, Cold Spring Harbor Laboratory Press, Cold Spring Harbor, N.Y.; Ausubel et al (1992) Current Protocols in Molecular Biology, Wiley and Sons, New York; Glover (1985) DNA Cloning I and II, Oxford Press; Anand (1992) Techniques for the Analysis of Complex Genomes (Academic Press); Guthrie and Fink (1991) Guide to Yeast Genetics and Molecular Biology, Academic Press; Harlow and Lane (1988) Antibodies: A Laboratory Manual, Cold Spring Harbor Laboratory, Cold Spring Harobor, NY; Jakoby and Pastan, eds. (1979) Cell Culture. Methods in Enzymology 58, Academic Press, Inc., Harcourt Brace Jaovanovich, NY; Nucleic Acid Hybridization (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal (1984), A Practical Guide To Molecular Cloning; Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Hogan et al. (1986) Manipulating the Mouse Embryo, Cold Spring Harbor Laboratory Press, Cold Spring Harbor, N.Y.

B. Capsid Modifications

Provided herein are modifications of the viral capsid that reduce or ablate the interaction of an adenovirus with a native receptors and optionally modifications that add interactions with targeted receptors. In particular, fiber modifications that result in reduction or ablation of the interaction of an adenovirus, particularly in vivo, with CAR. The adenovirus whose tropism is modified generally is one that in its native form interacts, particularly in vivo, with CAR. These fiber modifications can be combined with other capsid protein modifications, such as other fiber modifications and/or penton and/or hexon modifications, to ablate viral interactions with natural receptors, when expressed on a viral particle and/or to introduce interactions with targeted receptors. The modification should not disrupt trimer formation or transport of fiber into the nucleus.

1. Fiber Genes and Proteins

The fiber protein extends from the capsid and mediates viral binding to the cell surface by binding to specific cell receptors (Philipson et al. (1968) J. Virol. 2:1064-1075). The fiber is a trimeric protein that includes an N-terminal tail domain that interacts with the adenovirus penton base, a central shaft domain of varying length, and a C-terminal knob domain that contains the cell receptor binding site (Chroboczek et al., (1995) Curr. Top. Microbio. Immunol. 199:163-200; Riurok et al. (1990) J. Mol. Biol. 215:589-596; Stevenson et al. (1995) J. Virol. 69:2850-2857). The sequences of the fiber gene from a variety of serotypes including adenovirus serotypes 2 (Ad2), Ad5, Ad3, Ad35, Ad12, Ad40, and Ad41 are known. There are at least 21 different fiber genes in GENBANK.

As noted, the fiber protein can be divided into three domains (see, e.g., Green et al. (1983) EMBO J. 2:1357-1365). The conserved N-terminus contains the sequences responsible for association with the penton base as well as a nuclear localization signal. A rod-like shaft of variable length contains repeats of typically an about 15 amino acid β-structure, with the number of repeats ranging from about 6 to 23 (For example, 6 repeats in Ad3, 8 repeats in Ad37, 12 repeats in Ad4, 22 repeats in Ad5 and Ad41, and 23 repeats in Ad12). Often the last full repeat is followed by an incomplete repeat. For example, in Ad5 the last full repeat is the 21st repeat of the fiber shaft and this is followed by an incomplete repeat sequence (the 22nd repeat) before the junction with the fiber knob. A conserved stretch of amino acids that includes the sequence TLWT (SEQ ID No. 64 as exemplified) marks the boundary between the repeating units of β-structure in the shaft and the globular head domain, referred to as the knob. The C-terminal knob ranges in size from 157 amino acid residues for the short fiber of Ad41 to 193 residues for Ad11 and Ad34. The fiber spike is a homotrimer; the C-terminus is responsible for trimerization of the fiber homotrimer. There are typically 12 spikes per virion that are attached via association with the penton base complex.

The adenovirus fiber is a major determinant of adenovirus tropism. Fiber interacts with receptors on the cell surface to mediate viral binding to the cell surface. The primary receptor for most human adenoviruses is the Coxsackie-Adenovirus Receptor (CAR), a 46 kDa protein that is a member of the immunoglobulin superfamily (Bergelson et al., (1997) Science 275: 1320-1323). The receptor is distributed on many cell types in viva and is recognized by most Ad serotypes with the exception of subgroup B (Bergelson et al., (1997) Science 275: 1320-1323; Roelvink et al., (1998) J. Virol, 72: 589-596). The recognition between fiber and CAR occurs as an interaction of the fiber knob and CAR. Mutations in the fiber knob, such as in the loops joining the β-strands A and B or C and D (the AB- and CD-loops respectively) can substantially reduce and/or eliminate CAR binding (Roelvink et al., (1999) Science 286:1568-1571; Bewley et al., (1999) Science 286:1579-1583; Huang et al., (1999) J. Virol. 73:2798-2802). Adenoviruses having fiber knobs that interact with CAR include (a) adenoviruses of subgroup A, e.g., Ad12 (b) adenoviruses of subgroup C, e.g., Ad2 and Ad5 (c) adenoviruses of subgroup D including Ads 8, 9, 10, 13, 15, 17, 19 (including Ad19a and Ad19p), 20, 22-30, 32, 33, 36-39, and 42-49 and (d) adenoviruses of subgroup F, e.g., Ad40 and Ad41, specifically the short fiber of subgroup F.

Although the knob of fiber from most serotypes can recognize CAR, not all of these serotypes use CAR as their primary cellular receptor. Arnberg et al. ((2000) J. Virol. 74: 42-48) reported that Ad37, a serotype of subgroup D, uses a glycoprotein that contains sialic acid as its primary receptor on lung epithelial cells. Wu et al. ((2001) Virology 279: 78-89) demonstrated that Ad37 binds to an alternate receptor that is present on conjunctival cells, a cell type for which Ad37 and related subgroup D viruses Ad19a and Ad8 share an unusual tropism, and other cells. The knob of Ad37 fiber plays a role in this interaction, which can be disrupted by mutations in the CD loop of the Ad37 knob.

2. The Fiber Shaft

The fiber shaft also plays a role in cellular interactions. One example is the interaction of Adenovirus with hepatocytes where an entry pathway in vivo involves a mechanism mediated by the fiber shaft, such as Ad5 shaft, through heparin sulfate proteoglycans (HSP) binding. Elimination of this binding eliminates entry via HSP binding in hepatocytes. Such adenoviral fiber shaft modifications that ablate viral interaction with HSP are those such as described in U.S. Application Ser. No. 60/459,000 and incorporated herein by reference.

The ability of Adenovirus to interact with particular cell types also is influenced by modifications in the fiber shaft apart from those eliminating HSP binding. Adenovirus fiber shaft modifications that modify, reduce and/or eliminate cell binding are provided herein. Adenoviral fiber shaft modifications are provided that ablate or reduce interaction with CAR. Elimination of CAR binding eliminates cell binding and infection of CAR-dependent cell types such as lung epithelial cells. Ad fiber shaft modifications are provided that reduce or substantially eliminate CAR binding and ablate interaction with particular cell types, e.g. epithelial cells. Suitable modifications, such as those described herein, can be made with respect to any adenovirus in which the wildtype virus interacts with CAR.

The interaction of fiber or virus with CAR as well as cell binding and infectivity can be measured by any method known to those of skill in the art. One such assay is the measurement of cell infection using adenovirus particles or pseudotyped adenovirus particles expressing a marker protein such as GFP. Briefly, 50,000 adherent cells, such as A549 cells, are incubated with 20,000 virus particles for 3 hours at 37° C. After washing, the cells are analyzed by microscopy or fluorescence-assisted cell sorting (FACS) to distinguish infected cells which express GFP.

Another such assay for virus-cell interactions is a virus attachment assay. Briefly, cultured cells such as A549 cells, are detached and resuspended in phosphate-buffered saline (PBS) to a density of 1×10⁶ cells per tube. 1×10⁹ virus particles are added to the cells and the tubes are incubated with rocking at 4° C. to prevent virus internalization. To determine non-specific virus binding, samples are incubated with an excess of Ad5 knob protein. Cells are washed with PBS several times and then DNA is extracted by standard molecular biology methods known in the art. The presence of virus DNA is determined by methods such as PCR, Taqman, Southern blotting or any other methods known in the art.

Interaction of fiber or virus with CAR can be determined in cell based assays such as those described in Arnberg et al. (2000) J. Virology 74:42-48. Briefly, ³H-labeled virus particles are incubated with cells expressing CAR such as CHO-CAR cells, and with equivalent non-expressing CAR cells, such as CHO alpha2 (which express human α-2 integrin). A virus attachment assay is performed as described above or as in Arnberg et al. (2000) J. Virology 74:42-48. Scintillation counting is used to determine the amount of virus attached to the cells in the CAR expressing and non-CAR expressing samples. Virus particles that interact with CAR have increased attachment to cells expressing CAR as compared to the non-CAR expressing cells.

The interaction of fiber with CAR is determined by the fiber structure and influenced by the ability of fiber and the adenovirus particle orientation to the cell surface. Fiber length and flexibility are important determinate factors in cell interactions and infectivity. As described herein, particular modifications of the fiber shaft reduce cell interactions and infectivity by altering the structure or orientation of the fiber shaft or portions of the fiber shaft. These modifications alter interaction with CAR.

Fiber structure and orientation can be assessed by methods known in the art such as crystal structure analysis and cryo-electron microscopy (cryo-EM) studies (Xia et al., (1994) Structure 2:1259-1270; van Raaij et L, (2000) Structure 8:1147-1155; Stewart et al., (1997) EMBO J. 16:1189-1198). For example, as described herein, cryo-EM studies can be used to demonstrate the flexibility or rigidity of the fiber. Molecular and structural modeling software can then be used to construct images of adenoviral interactions at the cell surface, including particle orientations and receptor interactions. As described herein, modification of fiber shaft structure or orientation and the ability of adenovirus to interact with CAR is modified by replacing, mutating or deleting regions of the Ad fiber shaft.

3. Modifications of the Fiber Shaft

Provided herein are modified adenovirus fibers from serotypes that bind to CAR in vivo. The modified fibers include those that have modifications in the shaft. In particular, modifications of the β-repeats.

The Ad fiber shaft is made up of repeated units, referred to alternatively as repeats, β-repeats, pseudo-repeats or repeating motifs (each term is interchangeable). These repeats are approximately 13-20 (depending upon the alignment), generally about 15, amino acids in length each and occur in succession within the fiber shaft sequence. The repeats contribute the β-structure to the fiber shaft, with each repeat containing two β-strands separated by a β-turn. Fiber shafts from different adenoviruses have different numbers of repeat sequences. For example, Ad2 and Ad5 each have twenty-one complete repeats in the fiber shaft; whereas the fiber shaft of Ad37 has eight repeats. Generally adenoviruses with longer fibers interact with CAR; those with shorter fibers do not. It, however, is shown herein, that it is not only the length of the fiber that mediates interaction with CAR, but also the flexibility of the fiber, particular that which is mediated by the shaft. As shown herein, modification of the shaft repeats in CAR-binding fibers reduces the interaction in vivo.

It is shown herein that modifications of either of the β-strands or the β-turn of one or more repeats alters the structure of the fiber and substantially reduces or eliminates cell infectivity and interaction with CAR. Hence provided are modified fibers, particularly fibers with modified shafts, that have altered, interaction with CAR. The interaction can be modulated by altering these repeats, particularly, one or both of the repeats that correspond to the third β-repeat and/or the last full repeat of Ad2 or Ad5. For example, it is shown herein, that modification of one or both of the repeats that correspond to the third β-repeat and/or the last full repeat of Ad2 or Ad5, reduces binding to CAR. Modifications of fibers from other serotypes and types of adenoviruses can be similarly effected in order to modulate CAR interaction in vivo. This can be effected by eliminating repeats, inserting repeats, and modifying repeats. Particularly, modification of repeats corresponding to the third and last full repeat of Ad2 or Ad5 can modulate the CAR interaction in vivo. In addition or alternatively, the interaction can be modulated by deleting repeats in fibers that bind to CAR, and inserting them in fibers that do not bind to CAR.

For purposes herein, corresponding positions of repeats within different proteins can be determined by comparing and aligning fiber shaft sequences to maximize the number of amino acid residues and thus the number of aligned similar residues. Since the repeats are relatively short, this can be done manually. In aligning proteins such as the repeats of different fiber shafts, the entire fiber shaft or only a portion (also referred to as “region” herein) thereof can be used in the alignment. It is not necessary to use the entire fiber protein sequence nor the entire fiber shaft to sequence in the alignment. For example, FIGS. 1A and 1B show alignments of the third repeat sequences and the last full repeat sequences, respectively, for different adenovirus fiber proteins. Additionally, in aligning fiber proteins, not only are identical residues aligned but also conservative substitutions of amino acids. In a peptide or protein, suitable conservative substitutions of amino acids are known to those of skill in this art. Exemplary conservative substitutions are set forth in TABLE 1 above.

The alignment of identical and conserved amino acids in the fiber shaft regions is determined by standard alignment algorithms programs, used with default gap penalties established by each supplier. Manual alignment also can be used to maximize the number of aligned conservative and identical amino acids between proteins. Whether any two of more fiber shaft sequences or regions of fiber shafts, such as the repeats, alignment can be determined using known computer algorithms such as the “FAST A” program, using for example, the default parameters as in Pearson et al. (1988) Proc. Natl. Acad, Sci. USA 85:2444 (other programs include the GCG program package (Devereux, J., et al., Nucleic Acids Research 12(I):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar® “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps. As noted, in view of the relatively short regions in the fiber that are aligned, manual alignment can be adequate.

Similarly, using the techniques described herein and known in the art, comparison between regions within the same protein allows the identification of repeating motifs. For example, the alignment of regions within the Ad5 fiber shaft identifies 21 repeats each resembling a consensus sequence (SEQ ID No. 45), abCdEfGhijKlMno, where capitalized letters represent hydrophobic amino acids and the underlined residue (j) denotes the special proline or glycine that allows the β strands to form a β-turn.

The determination of corresponding repeats between adenovirus fiber proteins or within an adenovirus protein permits the modification of such repeats in any fiber, portion thereof or chimeric fiber protein. In another embodiment, repeats within fiber shaft regions and between fiber shaft regions are identified and one or more of these repeats is modified by mutating, deleting or replacing the repeat sequence. For example, at least one amino acid of one of the repeats within the shaft sequence is modified such that CAR interaction is substantially reduced or eliminated.

Modifications can be made by any methods known in the art. For example, PCR can be used to introduce specific mutations in the nucleic acid encoding a fiber protein. Alternatively, mutagenesis using chemical mutagens, ultraviolet wavelengths, mutagenic bacterial strains and mutagenic PCR protocols can be used to introduce one or more mutations in the nucleic acid encoding a fiber protein or a portion thereof. Mutations in the nucleic acid encoding a fiber protein introduce insertion of one or more amino acids, deletion of one or more amino acids or a change in the amino acid sequence in one or more of the repeats within the fiber shaft or any combination thereof.

Using assays such as the virus attachment, cell infectivity and CAR binding assays described herein and other such assays known in the art, the effect of the modifications on CAR binding and cell infectivity is assessed. Modification of the fibers as described herein is designed to result in a modification of the binding to CAR in vivo. In vitro assays, however, can be used to assess binding to CAR when modifications are made to the β-repeats, particularly repeats corresponding to the third and last full repeats of Ad2 or Ad5.

Generally the desired modifications are those of CAR-binding fibers to eliminate or reduce (by at least 2-fold, generally 10-fold, 100-fold or more) CAR binding. As shown herein this is achieved by modifying the β-repeats (replacing selected repeats with corresponding repeats from non-CAR binding fibers, altering one or more amino acids in a repeat, particularly the repeat corresponding to the third and last full repeat of Ad2 or Ad5) and/or eliminating repeats, such as one up to all of the fourteen central repeats in such CAR-binding fibers. It also is understood that fibers that generally do not bind to CAR, such as Ad37 can be modified to bind to CAR by adding repeats and/or replacing repeats, particularly a repeat corresponding to the third or last full repeat, with those from a CAR-binding fiber.

Among the modified fibers provided herein are those in which the tertiary structure of modified fiber is altered compared to the structure of the unmodified fiber such that the modified fiber is more rigid than the unmodified fiber. Included are modified fibers that are shortened or exhibit reduced flexibility compared to the unmodified fiber. The fiber shaft is modified such that one or more of the repeats are modified. As described herein, the Ad5 fiber sequence (SEQ ID No. 35) is used as a reference sequence. Thus, for example, “modifications of the third repeat” means modification of the third repeat of Ad5 or modification to a repeat corresponding to the third repeat of Ad5. The “corresponding repeat” may not be the third in a sequence of repeats in another fiber, portion thereof or chimera. For example, the sequence of amino acids corresponding to the third repeat within the fiber shaft sequence is modified. For example, the fiber shaft of a subgroup C fiber, e.g. Ad2 or Ad5, is modified to mutate, replace, insert or delete at least one of the amino acids within the sequence of the third repeat in Ad2 and Ad5 (SEQ ID NOS: 42 and 43) and thus substantially reduce or eliminate CAR binding. For example, the TTVT/S sequence (SEQ ID No. 44) within the 3rd repeat is deleted, replaced, or one or more amino acids inserted by standard molecular biology and biochemistry methods known to those skilled in the art. Modifications in the third repeat at this locus (or a corresponding locus) disrupt the structure of the fiber shaft, for example as described herein, resulting in fibers with increased rigidity and decreased the flexibility of the fiber shaft.

As another example, the sequence of amino acids corresponding to the last full repeat in the fiber shaft is modified. For example, the KLGXGLXFD/N motif (SEQ ID No. 49), found in the last full repeat of the fiber shaft of most serotypes is modified by mutating, replacing, inserting or deleting at least one amino acids within the motif or inserting at least one additional amino acid into the motif and thus CAR binding is substantially reduced or eliminated. For example, the fiber shaft of a subgroup C fiber, e.g. Ad2 or Ad5, is modified to mutate, replace or delete the amino acid sequence of the last repeat, the 21st repeat of Ad2 or Ad5 (SEQ ID NOS: 46 and 47), for example, the KLGXGLXFD/N motif (SEQ ID No. 49), is deleted or mutated by standard molecular biology and biochemistry methods known to those skilled in the art.

Modification of the last repeat in the fiber shaft alters the structure of the fiber shaft. A hinge structure exists at the interface of the knob and shaft in the Ad2 fiber (van Raaij et al., (2000) Nature 401:935-938). For example, modification to the last repeat alters the structure of the fiber shaft and can alter the hinge structure between the fiber shaft and knob. Fibers with such modifications are provided.

As noted, modified fibers provided herein include those in which at least one of the repeat regions of the fiber shaft is replaced with the repeat sequence from an Ad fiber that does not bind CAR or does not use CAR as its primary receptor. Such regions can be derived from Ad serotypes of subgroup D such as Ads 8, 9, 10, 13, 15, 17, 19 (including Ad19a and Ad19p), 20, 22-30, 32, 33, 36-39, and 42-49. For example, the fiber shaft of a CAR-binding fiber such as that of subgroup C, e.g. Ad2 or Ad5, is modified to replace one of the repeats with a corresponding repeat sequence of subgroup D, such as Ad37, Ad8, Ad9 or A19.

In other exemplary embodiments, amino acids of the region corresponding to the third repeat within the fiber shaft sequence of a fiber that binds CAR is replaced with the third repeat from a fiber shaft from an Ad that does not bind CAR or does not use CAR as its primary receptor. For example, the fiber shaft of a subgroup C fiber, e.g. Ad2 or Ad5, is modified to replace the amino acid of the third repeat in Ad2 or Ad5 (SEQ ID NOS: 42 and 43) with the third repeat (or repeat that corresponds to the third repeat) from serotype D virus fiber shaft. Such substitution reduces or eliminates CAR binding. For example, the third repeat of Ad5 fiber shaft (SEQ ID No. 43) is replaced with the third repeat of the Ad37 fiber shaft (SEQ ID No. 58) by standard molecular biology and biochemistry methods known to those skilled in the art.

In another aspect of this embodiment, the repeat corresponding to the third repeat within the fiber shaft sequence of a fiber that binds CAR is modified by replacing one or more amino acids of the third repeat with the corresponding amino acid from a fiber that does not bind CAR (or use it as its primary receptor in vivo). The third repeat of a fiber shaft from a CAR binding fiber such as a subgroup C fiber, e.g. Ad2 or Ad5, is modified to replace one or more amino acids, up to all of the amino acids in the third repeat of Ad2 or Ad5 (SEQ ID NOS: 42 and 43) with the corresponding amino acids from the third repeat of a fiber shaft such as from Ad 37, Ad8, Ad9, or Ad15 (SEQ ID NOS: 58, and 66-68) by standard molecular biology and biochemistry methods known to those skilled in the art, and thus substantially reduce or eliminate binding to CAR.

In another aspect of this embodiment, the region corresponding to the third repeat within the fiber shaft sequence of a fiber that binds to CAR is modified by replacing one or more amino acids with a non-conservative amino acid substitution (conservative amino acid substitutions are those such as provided in Table 1, above) by standard molecular biology and biochemistry methods known to those skilled in the art, and thus substantially reduce or eliminate binding to CAR. For example, one or more of the amino acids of TTVT/S motif (SEQ ID No. 44) is mutated to a nonconservative amino acid, such as proline and CAR binding is substantially reduced or eliminated. For modified fibers that contain a single modification in the shaft that reduces CAR binding, the modification includes a modification of at least one nucleotide in the region corresponding to the TTVT/S motif exemplified herein, whereby CAR binding is reduced or eliminated.

In another aspect of this embodiment, the region corresponding to the last full repeat in the fiber shaft is modified. For example, the KLGXGLXFD/N motif (SEQ ID No. 49), found in the last full repeat of the fiber shaft of most serotypes, is modified by replacing this repeat with the last full repeat of a fiber shaft from a fiber that does not have this motif. The last full repeat of a fiber shaft from a CAR binding fiber such as a subgroup C fiber, e.g. Ad2 or Ad5, is modified to replace the 21st repeat of Ad2 or Ad5 (SEQ ID NOS: 46 and 47) with the corresponding last full repeat of a fiber shaft that does not contain this motif, such as from Ad 37, Ad8, Ad9, or Ad15 (SEQ ID NOS: 48, and 59-61) by standard molecular biology and biochemistry methods known to those skilled in the art, and thus substantially reduce or eliminate binding to CAR.

In another aspect of this embodiment, the region corresponding to the KLGXGLXFD/N motif (SEQ ID No. 49), found in the last full repeat of the fiber shaft of most serotypes, is modified by replacing one or more amino acids with the corresponding amino acid from a fiber that does not have this motif. The last full repeat of a fiber shaft from a CAR binding fiber such as a subgroup C fiber, e.g. Ad2 or Ad5, is modified to replace one or more amino acids, up to all of the amino acids the KLGXGLXFD/N motif (SEQ ID No. 49) in the 21st repeat of Ad2 or Ad5 (SEQ ID NOS: 46 and 47) with the corresponding amino acids from the last full repeat of a fiber shaft that does not contain the polyeptide with this motif, such as from Ad 37, Ad8, Ad9, or Ad15 (SEQ ID NOS: 48, and 59-61), thereby reducing or eliminating binding to CAR. Modification can be effected by known standard molecular biology and biochemistry methods known to those skilled in the art in another aspect of this embodiment, a region corresponding to the KLGXGLXFD/N motif (SEQ ID No. 49), that occurs in the last full repeat of the fiber shaft of most serotypes, is modified by replacing one or more amino acids with a non-conservative amino acid substitution (conservative amino acid substitutions are those such as provided in Table 1, above).

In another embodiment, fiber shafts with a plurality of modifications, particularly if more than one repeat are provided. For example, modifications in repeats corresponding to the 3rd repeat of the shaft and the last full repeat of the shaft are provided. For example, the 3rd repeat is modified by mutating, replacing, inserting or deleting at least one amino acid of the repeat and the last full repeat also is modified by replacement, mutation, insertion or deletion of at least one amino acid within the repeat, such that the fiber structure is altered and the fiber interaction with CAR is substantially reduced or eliminated. In another aspect of this embodiment, the third repeat (or repeat corresponding to the third repeat) and last full repeat of a fiber shaft from a CAR-interacting fiber are replaced with the corresponding repeats of a fiber shaft from a fiber that does not interact with CAR. For example, the fiber shaft of a subgroup C fiber, e.g. Ad2 or Ad5, is modified to replace the amino acid sequence of the third repeat in Ad2 and Ad5 (SEQ ID NOS: 42 and 43) with the corresponding repeat sequence of a subgroup D virus fiber shaft and the last full repeat of the fiber shaft also is modified to replace the 21st repeat of Ad2 or Ad5 (SEQ ID NOS: 46 and 47) with the last full repeat of a fiber shaft that does not contain this motif, such as from Ad37, Ad8, Ad9, or Ad15 (SEQ ID NOS: 48, and 59-61). This alters the structure of the fiber and reduces or eliminates CAR binding. An exemplary chimeric fiber is the Ad5s/Ad37s fiber (SEQ ID No. 55) depicted schematically in FIG. 2, where the 3rd and 21st repeats of Ad5 fiber are replaced with the corresponding repeats of Ad37.

In another embodiment, one or more repeats is modified such that one or more of the amino acids corresponding to the consensus sequence (SEQ ID No. 45), abCdEfGhijKlMno, is modified such that the fiber structure is altered. For example, one or more of the conserved hydrophobic residues is deleted or replaced with a non-hydrophobic amino acid such as known in the art such that the fiber structure is altered and CAR binding is reduced or eliminated. Fiber structure can be assessed by any of the methods described herein or known in the art. In another example, the conserved proline or glycine denoted by j in SEQ ID No. 45 is deleted or replaced with a non-conservative amino acid change such that the β-turn formed by this amino acid is disrupted and the resulting modified fiber interaction with CAR is substantially reduced or eliminated.

In another embodiment, one or more repeats or a portion of one or more repeats is deleted, thus altering the fiber structure and its interaction with CAR. For example, repeats are deleted from the Ad5 fiber shaft resulting in reduced cell infectivity One example of such a fiber is Ad5As, which has a deletion of the 14 central repeats (SEQ ID No. 51).

### Combinations of a Plurality of Modification

The modifications provided herein can be combined with other fiber modifications, and in the viral particle other capsid modifications, to further detarget and/or retarget the resulting particle that expresses the capsid proteins. For example, additional modifications that reduce binding to CAR can be combined with those provided herein to further reduce or eliminate CAR binding. Other modifications that reduce binding to other receptors and proteins also can be introduced. For example, fiber shaft modifications that reduce CAR interaction as described herein can be combined with modifications that reduce HSP interaction. Suitable adenovirus fiber shaft modifications include modification of the HSP binding motif of the fiber protein such that it no longer interacts with HSP on the cell surfaces, particularly hepatocytes, such as those described in U.S, patent application Ser. No. 10/351,890. For example, where the adenoviral fiber is from a subgroup C adenovirus, binding to HSP can be eliminated or reduced by mutating the fiber shaft in order to modify the HSP binding motif, which is, for example, the KKTK sequence (SEQ ID No. 65) located between amino acid residues 91 to 94 in the Ad 5 fiber (SEQ ID No. 35). The ability of a fiber to interact with HSP is modified by replacing the wild-type fiber shaft with a fiber shaft, or portion thereof, of an adenovirus that does not interact with HSP to produce chimeric fiber proteins. The portion is sufficient to reduce or eliminate interaction with HSP. Examples of adenoviruses having fiber shafts that do not interact with HSP include (a) adenoviruses of subgroup B, such as, but are not limited to, Ad3, Ad35, Ad7, Ad11, Ad16, Ad21, Ad34 (b) adenoviruses of subgroup F, such as, but are not limited to, Ad40 and Ad41, specifically the short fiber, and (c) adenoviruses of subgroup D, such as but are not limited to, Ad 19a and Ad 19p.

In another embodiment, fiber shaft modifications that reduce CAR interaction as described are combined with adenoviral fiber modifications made by replacing the wild-type fiber knob with a fiber knob of an adenovirus that does not interact with CAR. Examples of adenoviruses having fiber knobs that do not interact with CAR include (a) adenoviruses of subgroup B, e.g., Ad3, Ad35, Ad7, Ad11, Ad16, Ad21, Ad34, (b) adenoviruses of subgroup F, e.g., Ad40 and Ad41, specifically the short fiber. Additional mutations and fibers that have altered CAR interaction are described in U.S. application Ser. No. 10/351,890, for example, the K01 and K012 mutants.

Capsids in viral particles that express fibers with modifications that reduce viral interaction with CAR as described herein can be combined with penton modifications that reduce viral interactions with αᵥ integrins. Suitable adenoviral penton modifications include the penton modifications, which are known to those of skill in the art (see, e.g., U.S. Pat. No. 5,731,190; see, also Einfeld et al. (2001) J. Virology 75:11284-11291; and Bai et al. (1993) J. Virology 67:5198-5205). For example, penton interaction with a, integrins can be reduced or even eliminated by substitution of the RGD tripeptide motif, required for αᵥ interaction, in penton with a different tripeptide that does not interact with an αᵥ integrin. The penton proteins with reduced αᵥ integrin interactions are modified by chemical and biological techniques known to those skilled in the art (see, e.g., described U.S. Pat. No. 6,731,190). Generally, the adenovirus is a subgroup B or C adenovirus.

Also provided are other fiber modifications that alter the tropism of the adenovirus. Adenovirus fiber modifications are made that detarget the virus particles in combination with modifications that retarget the particles to specific cell types. For example, a chimeric fiber is provided that joins a portion of a fiber that recognizes cell surface receptors on photoreceptor cell types, such as the fiber knob or portion thereof from Ad37 (see U.S. application Ser. No. 09/562,934; see, also corresponding published application US200020193327), with the remaining portion the fiber provided from a fiber that does not efficiently interact with these cell types. Recombinant viral particles for targeting therapeutic products to these cells can be constructed with these chimeric fibers to treat such degenerative ocular diseases, such as, but not limited to, retinitis pigmentosa, Stargardt's disease, diabetic retinopathies, retinal vascularization, and others that have genetic bases. Genes expressed in the photoreceptor cells at the back of the retina are implicated in these diseases. In one aspect of this embodiment, the fiber shaft of the chimeric fiber is further modified such that it no longer binds CAR efficiently, for example by mutating, deleting or replacing one of the repeats within the fiber shaft.

Chimeric fibers are provided that target dendritic cells (DCs). The role of DCs in enhancing antigen-specific immune responses is known. DCs can be exploited to aid in vaccination against autoimmunity, allergy and transplantation rejection, all of which result from an uncontrolled or unchecked immune response (Hawiger et al. (2001) J. Exp. Med. 194:769-779; Steinman et al. (2003) Annual Rev. Immunol. 27:685-711). Vaccine strategies involving DCs can be important for the treatment of a variety of clinically important autoimmune and related diseases, including systemic lupus erythematosus, myasthenia gravis, rheumatoid arthritis, insulin-dependent diabetes mellitus and Graves' disease. Recombinant adenoviruses with fiber proteins from the Subgroup B viruses Ad 16 and Ad35 have been found to have an increased ability to infect human DC (Havenga et al. (2002) J. Virol. 76:4612-4620; Rea et al. (2001) J. Immunol. 166:5236-5244). Recombinant adenovirus particles are constructed with fiber or a portion thereof from an adenovirus that targets dendritic cells (see U.S. provisional application Ser. No. 60/467,500) and the fiber is further modified such that it no longer binds CAR efficiently, for example by mutating, deleting or replacing one of the repeats within the fiber shaft. In these adenoviral particles, the adenoviral (Ad) particle, except for the fiber, is from a subgroup C adenovirus; and the fiber includes a sufficient portion of an adenovirus Subgroup D, such as Ad19p, to target receptors on dendritic cells. The adenoviral particles with the modified fibers also are constructed to express therapeutic products to be expressed in dendritic cells such as tumor antigens. The adenoviral particles can include heterologous nucleic acid encoding a product for expression in a dendritic cell for presentation or to alter the activity of the dendritic cell. Exemplary heterologous products include, but are not limited to, tumor antigens (see Table in Section F below) and other immune modulating proteins.

C. Nucleic acids, Adenoviral Vectors and Cells Containing the Nucleic Acids and Cells Containing the Vectors

1. Adenoviral Vectors and Particles

The adenovirus vector genome that is encapsulated in the virus particle and that expresses exogenous genes is a component of the system. Components of a recombinant adenovirus vector genome include the ability to express selected adenovirus structural genes, to express a desired exogenous protein, and to contain sufficient replication and packaging signals that the genome is packaged into a gene delivery vector particle. An exemplary replication signal is an adenovirus inverted terminal repeat containing an adenovirus origin of replication, as is well known and described herein.

Although adenoviruses encode many proteins, not all adenovirus proteins are required for assembly of a recombinant adenovirus particle. Deletion of the appropriate genes from a recombinant Ad vector permits accommodation of even larger “foreign” DNA segments.

Adenovirus particles for delivery of heterologous nucleic acids to cells in vitro and in vivo, including those for human therapy, are known. Such known viruses can be modified as provided herein to reduce or eliminate interaction with CAR and optionally to target selected receptors to retarget to cells expressing such receptors. The adenoviral vectors that are used to produce the viral particles can include other modifications. Modifications include modifications of the adenovirus genome that is packaged in the particle in order to make an adenoviral vector. As discussed above, adenovirus vectors and particles with a variety of modifications are available. Modifications of adenoviral vectors include deletions known in the art, such as deletions in one or more of the E1a, E2a, E2b, E3, or E4 coding regions. These adenoviruses are sometimes referred to as early generation adenoviruses and include those with deletions of all of the coding regions of the adenoviral genome (“gutless” adenoviruses, discussed below) and also include replication-conditional adenoviruses, which are viruses that replicate in certain types of cells or tissues but not in other types as a result of placing adenoviral genes essential for replication under control of a heterologous promoter (see, also U.S. Pat. No. 5,998,205, U.S. Pat. No. 5,801,029; U.S. patent application 60/348,670 and corresponding published International PCT application No. WO02/06786). These include the cytolytic, cytopathic viruses (or vectors), including the oncolytic viruses.

Alternatively, the vector can include a mutation or deletion in the E1 b gene. Typically such mutation or deletion in the E1 b gene is such that the E1 b-19 kD protein becomes non-functional. This modification of the E1 b region can be combined with vectors where all or a part of the E3 region is present.

The oncolytic adenoviral vector can further include at least one heterologous coding sequence, such as one that encodes a therapeutic product. The heterologous coding sequence, such as therapeutic gene, is generally, although not necessarily, in the form of cDNA, and can be inserted at any locus that does not adversely affect the infectivity or replication of the vector. For example, it can be inserted in an E3 region in place of at least one of the polynucleotide sequences that encode an E3 protein, such as, for example, the 19 kD or 14.7 kD E3 gene.

a. Gutless Vectors

Gutless adenovirus vectors are those from which most or all viral genes have been deleted. They are grown by co-infection of cells with a “helper” virus (such as using an E1-deleted Ad vector), where the packaging cells expresses the E1 gene products. The helper virus trans-complements the missing Ad functions, including production of the viral structural proteins needed for particle assembly.

Adenovirus DNA also includes inverted terminal repeat sequences (ITRs) ranging in size from about 100 to 150 bp, depending on the serotype. The inverted repeats permit single strands of viral DNA to circularize by base-pairing of their terminal sequences to form base-paired “panhandle” structures that are required for replication of the viral DNA. For efficient packaging, the ITRs and the packaging signal (a few hundred bp in length) contains the “minimum requirement” for replication and packaging of a genomic nucleic acid into an adenovirus particle. Helper-dependent vectors lacking all or most viral ORFs but including these essential cis elements (the ITRs and contiguous packaging sequence) have been constructed.

To incorporate the capsid modifications into a gutted adenoviral vector capsid, the changes must be made to the helper virus as described herein. All the necessary Ad proteins including the modified capsid protein are provided by the modified helper virus and/or the packaging cells, and the gutted adenovirus particles are equipped with the particular modified capsid as expressed in the host cells. The E1a, E1b, E2a, E2b and E4 are generally required for viral replication and packaging. If these genes are deleted, then the packaging cell or helper virus must provide these genes or functional equivalents.

A helper adenovirus vector genome and a gutless adenoviral vector genome are delivered to packaging cells. The cells are maintained under standard cell maintenance or growth conditions, whereby the helper vector genome and the packaging cell together provide the complementing proteins for the packaging of the adenoviral vector particle. Such gutless adenoviral vector particles are recovered by standard techniques. The helper vector genome can be delivered in the form of a plasmid or similar construct by standard transfection techniques, or it can be delivered through infection by a viral particle containing the genome. Such viral particle is commonly called a helper virus. Similarly, the gutless adenoviral vector genome can be delivered to the cell by transfection or viral infection.

The helper virus genome can be the modified adenovirus vector genome as disclosed herein. Such genome also can be prepared or designed so that it lacks the genes encoding the adenovirus E1A and E1B proteins. In addition, the genome can further lack the adenovirus genes encoding the adenovirus E3 proteins. Alternatively, the genes encoding such proteins can be present but mutated so that they do not encode functional E1A, E1B and E3 proteins. Furthermore, such vector genome can not encode other functional early proteins, such as E2A, E2B3, and E4 proteins. Alternatively, the genes encoding such other early proteins can be present but mutated so that they do not encode functional proteins.

In producing the gutless vectors, the helper virus genome also is packaged, thereby producing helper virus. In order to minimize the amount of helper virus produced and maximize the amount of gutless vector particles produced, the packaging sequence in the helper virus genome can be deleted or otherwise modified so that packaging of the helper virus genome is prevented or limited. Since the gutless vector genome will have an unmodified packaging sequence, it will be preferentially packaged.

One method is to mutate the packaging sequence by deleting one or more of the nucleotides comprising the sequence or otherwise mutating the sequence to inactivate or hamper the packaging function. One exemplary approach is to engineer the helper genome so that recombinase target sites flank the packaging sequence and to provide a recombinase in the packaging cell. The action of recombinase on such sites results in the removal of the packaging sequence from the helper virus genome. The recombinase can be provided by a nucleotide sequence in the packaging cell that encodes the recombinase. Such sequence can be stably integrated into the genome of the packaging cell. Various kinds of recombinase are known by those skilled in the art, and include, but are not limited to, Cre recombinase, which operates on so-called lox sites, which are engineered on either side of the packaging sequence as discussed above (see, e.g., U.S. Pat. Nos. 5,919,676, 6,080,569 and 5,919,676; see, also, e.g., Morsy and Caskey, Molecular Medicine Today, January 1999, pgs. 18-24).

An example of a gutless vector is pAdARSVDys (Haecker et al. (1996) Hum Gene Ther. 7:1907-1914)). This plasmid contains a full-length human dystrophin cDNA driven by the RSV promoter and flanked by Ad inverted terminal repeats and packaging signals. 293 cells are infected with a first-generation Ad, which serves as a helper virus, and then transfected with purified pAdARSVDys DNA. The helper Ad genome and the pAdARSVDys DNA are replicated as Ad chromosomes, and packaged into particles using the viral proteins produced by the helper virus. Particles are isolated and the pAdARSVDys-containing particles separated from the helper by virtue of their smaller genome size and therefore different density on CsCl gradients. Other examples of gutless adenoviral vectors are known (see, e.g., Sandig et al., (2000) Proc. Natl. Acad. Sci. U.S.A. 97(3):1002-7).

b. Oncolytic Vectors

Briefly, oncolytic adenoviruses, which are viruses that replicate selectively in tumor cells, are designed to amplify the input virus dose due to viral replication in the tumor, leading to spread of the virus throughout a tumor mass. In situ replication of adenoviruses leads to cell lysis. This in situ replication permits relatively low, non-toxic doses to be highly effective in the selective elimination of tumor cells. One approach to achieving selectivity is to introduce loss-of-function mutations in viral genes that are essential for growth in non-target cells but not in tumor cells. (See, e.g., U.S. Pat. No. 5,801,029.) This strategy is exemplified by the use of AddI1520, which has a deletion in the E1b-55 KD gene. In normal cells, the adenoviral E1b-55 KD protein is needed to bind to p53 to prevent apoptosis. In p53-deficient tumor cells, E1 b-55K binding jto p53 is unnecessary. Thus, deletion of E1b-55 KD should restrict vector replication to p53-deficient tumor cells.

Another approach is the use of tumor-selective promoters to control the expression of early viral genes required for replication (see, e.g., International PCT application Nos. WO 96/17053 and WO 99/25860). In this approach, adenoviruses selectively replicate and lyse tumor cells if the gene that is essential for replication is under the control of a promoter or other transcriptional regulatory element that is tumor-selective.

For example oncolytic adenoviral vectors that contain a cancer selective regulatory region operatively linked to an adenoviral gene essential for adenoviral replication are known (see, e.g., U.S. Pat. No. 5,998,205). Adenoviral genes essential for replication include, but are not limited to, E1a, E1b, E2a, E2b and E4. Examples of cancer selective regulatory regions include the promoters and/or enhancers from carcinoembryonic antigen (CEA), DE3 breast cancer-specific sequences, alpha-feroprotein, Erb-B2 and tyrosinase. For example, an exemplary oncolytic adenoviral vector has a cancer selective regulatory region operatively linked to the E1a gene. In other embodiments, the oncolytic adenoviral vector has a cancer selective regulatory region such as one of those described above, operatively linked to the E1a gene and a second cancer selective regulatory region operatively linked to the E4 gene. The vectors also can include at least one therapeutic transgene, such as, but not limited to, a polynucleotide encoding a cytokine such as GM-CSF that can stimulate a systemic immune response against tumor cells.

Other exemplary oncolytic adenoviral vectors include those in which expression of an adenoviral gene, which is essential for replication, is controlled by E2F-responsive promoters, which are selectively transactivated in cancer cells. Thus, vectors that contain an adenoviral nucleic acid backbone that contains in sequential order: A left ITR, an denoviral packaging signal, a termination signal sequence, an E2F responsive promoter which is operably linked to a first gene, such as E1a, essential for replication of the recombinant viral vector and a right ITR (see, published International PCT application No. WO02/06786, and U.S. Pat. No. 5,998,205).

c. Helper Independent Viruses

Contemplated for use are helper-independent fiberless recombinant adenovirus vector genomes that include genes that (a) express all or most adenovirus structural gene products (b) contain an adenovirus packaging signal and inverted terminal repeats containing adenovirus origin of replication and (c) can express an exogenous protein, such as a marker protein or therapeutic protein as described herein.

The adenovirus vector can be constructed to express fiber protein or a portion thereof or a chimeric fiber protein. For example, viral backbones with the modified fibers as described herein are substituted in place of the Ad5 fiber gene are constructed. One such system for expression is based on the pAdEasy plasmid (see U.S. Pat. No. 5,922,576, U.S. Application Ser. No. 60/459,000, and also He et al., (1998) Proc. Natl. Acad. Sci. 2509-2514). This system includes a large plasmid (pAdEasy) that contains most of the Ad5 genome and smaller shuttle plasmids with the left end of the viral genome, including an E1 deletion and polylinker for insertion of transgenes. Recombination between pAdEasy and a shuttle plasmid in E. coli reconstitutes a full-length infectious Ad genome. Additional recombinations of constructed vectors with the shuttle plasmid pAdTrack, which contains a CMV-driven EGFP reporter gene (He et al., Proc. Natl. Acad. Sci. USA 95:2509-2514 (1998); U.S. Pat. No. 5,922,576) results in Ad vectors with the EGFP reporter at the site of the E1 deletion and as well as the modified fiber gene in the viral chromosome. The EGFP reporter can be used to monitor viral infectivity, biodistribution and tropism as described herein and by other methods known in the art.

The adenovirus vector genome is propagated in the laboratory in the form of rDNA plasmids containing the genome, and upon introduction into an appropriate host, the viral genetic elements provide for viral genome replication and packaging rather than plasmid-based propagation. Exemplary methods for preparing an Ad-vector genome are described in the Examples.

A vector herein includes a nucleic acid (typically DNA) molecule capable of autonomous replication in a cell and to which a DNA segment, e.g., a gene or polynucleotide, can be operatively linked to bring about replication of the attached segment. For purposes herein, one of the nucleotide segments to be operatively linked to vector sequences can encode at least a portion of a therapeutic nucleic acid molecule. As noted herein, therapeutic nucleic acid molecules include those encoding proteins and also those that encode regulatory factors that can lead to expression or inhibition or alteration of expression of a gene product in a targeted cell.

The Ad vector also can be constructed such that it does not express fiber or expresses insufficient adenovirus fiber protein to package a fiber-containing adenovirus particle without complementation of a fiber gene such as from a packaging cell line, for example the packaging cell lines as described below.

2. Packaging and Complementing Cell Lines

The viral particles provided herein can be made by any method known to those of skill in the art. Generally they are prepared by growing the adenovirus vector that contains nucleic acid that encodes the modified fiber protein in standard adenovirus packaging cells to produce particles that express the modified fibers. Alternatively, the vectors do not encode fibers. Such vectors are packaged in cells that express the modified fiber proteins to produce particles.

As discussed, recombinant adenoviral vectors generally have at least a deletion in the first viral early gene region, referred to as E1, which includes the E1a and E1b regions. Deletion of the viral E1 region renders the recombinant adenovirus defective for replication and incapable of producing infectious viral particles in subsequently-infected target cells. Thus, to generate E1-deleted adenovirus genome replication and to produce virus particles requires a system of complementation that provides the missing E1 gene product. E1 complementation is typically provided by a cell line expressing E1, such as the human embryonic kidney packaging cell line, i.e. an epithelial cell line, called 293. Cell line 293 contains the E1 region of adenovirus, which provides E1 gene region products to “support” the growth of E1-deleted virus in the cell line (see, e.g., Graham et al., J. Gen. Virol. 36: 59-71, 1977). Additionally, cell lines that can be usable for production of defective adenovirus having a portion of the adenovirus E4 region can be employed (see, e.g., International PCT application No. WO 96/22378). Multiply deficient adenoviral vectors and complementing cell lines have also been described (see WO 95/34671 and also, U.S. Pat. No. 5,994,106).

For example, copending U.S. application Ser. No. 09/482,682, published as US20030157688 (see, also International PCT application No. WO/0042208) provides packaging cell lines that support viral vectors with deletions of major portions of the viral genome, without the need for helper viruses and also provides cell lines and helper viruses for use with helper-dependent vectors. The packaging cell line has heterologous DNA stably integrated into the chromosomes of the cellular genome. The heterologous DNA sequence encodes one or more adenovirus regulatory and/or structural polypeptides that complement the genes deleted or mutated in the adenovirus vector genome to be replicated and packaged.

Packaging cell lines express, for example, one or more adenovirus structural proteins, polypeptides, or fragments thereof, such as penton base, hexon, fiber, polypeptide IIIa, polypeptide V, polypeptide VI, polypeptide VII, polypeptide VIII, and biologically active fragments thereof. The expression can be constitutive or under the control of a regulatable promoter. These cell lines are particularly designed for expression of recombinant adenoviruses intended for delivery of therapeutic products. For use herein, such packaging cell lines can express the modified capsid proteins, such as the fiber proteins when binding to CAR is reduced or eliminated, and/or the modified penton and hexon proteins.

Particular packaging cell lines complement viral vectors having a deletion or mutation of a DNA sequence encoding an adenovirus structural protein, regulatory polypeptides E1A and E1B, and/or one or more of the following regulatory proteins or polypeptides: E2A, E2B, E3, E4, L4, or fragments thereof.

The packaging cell lines are produced by introducing each DNA molecule into the cells and then into the genome via a separate complementing plasmid or plurality of DNA molecules encoding the complementing proteins can be introduced via a single complementing plasmid. Of interest herein, is a variation in which the complementing plasmid includes DNA encoding adenovirus fiber protein, a chimeric fiber or modified variant thereof.

For applications, such as therapeutic applications, the delivery plasmid further can include a nucleic acid encoding a heterologous polypeptide. Exemplary delivery plasmids include, but are not limited to, application Ser. No. 09/562,934; see, also corresponding published application US200020193327). In a similar or analogous manner, therapeutic nucleic acids, such as nucleic acids that encode therapeutic products, can be introduced.

The cell further includes a complementing plasmid encoding a fiber as contemplated herein; the plasmid or portion thereof is integrated into a chromosome(s) of the cellular genome of the cell.

Typically, the packaging cell lines will contain nucleic acid encoding the fiber protein or modified protein stably integrated into a chromosome or chromosomes in the cellular genome. The packaging cell line can be derived from a prokaryotic cell line or from a eukaryotic cell line. While mammalian cells, particularly epithelial cell lines, such as the 293, A549, and AE1-2a cell lines, are exemplified, a variety of other non-epithelial cell lines can be used in various embodiments. Any other cell lines suitable for such use are contemplated herein.

D. Detargeting

The fiber modifications provided herein permit detargeting of adenoviral particles by reducing or eliminating interaction of serotypes, such as the serotype C viruses with CAR. Hence particles that express fibers with alterations in the shaft, particularly in the β-repeat region as described herein, exhibit reduced CAR interaction. The fiber modifications described herein can be combined with other modifications to further reduce any CAR interaction and/or to detarget from additional receptors. For example, interaction of Ad particles with hepatocytes can be reduced or eliminated to thereby reduce liver toxicity in adeno-viral-mediated therapy. Ablation of liver transduction can require combinations of modification(s) to the adenovirus particle (see U.S. application Ser. Nos. 10/351,890, 60/459,000). A method for reducing liver toxicity in adenoviral-mediated therapy includes modifying an adenoviral vector to ablate native tropism to liver cells in vivo. Such vectors can be administered to a subject. Such modifications include the modifications described herein.

Such detargeted Ad vectors can be constructed, for example, with adenoviral vectors in which the fiber shaft's interaction with HSP (Heparin Sulfate Proteoglycans; also referred to as heparin sulfate glycosaminoglycans) is ablated (reduced or substantially eliminated), particularly in vivo, combined with modification to the fiber shaft repeats as described herein. Mutations such as those described in U.S. Application Ser. No. 60/459,000 are made to the HSP binding site in the fiber, for example to the KKTK consensus sequence (SEQ ID No. 65) in Ad2 and Ad5 can be introduced to reduce HSP interaction. The mutation to the HSP binding site is combined with mutations to the fiber shaft repeats described herein. Mutations are effected using techniques known in the art such as overlap PCR and PCR SOEing or other known techniques such as homologous recombination and chemical mutagenesis. The modified fibers are then expressed and incorporated into adenoviral particles by methods such as those described herein. Combination of the modifications of the fiber shaft repeats and the HSP binding site serve to further detarget the adenoviral particles.

Modifications in the fiber shaft are also provided in combination with fiber knob modifications that ablate viral interaction with CAR. The fiber knob modifications include: (a) mutations of individual amino acids in the fiber loop that interact with CAR, such as, for example, AB or CD loop modifications; (b) mutations of individual amino acids in the fiber loop that modify the ability of the CAR binding motif to interact with CAR; and (c) replacements of fiber knobs using adenoviruses that do not interact with CAR, such as, for example, Ad3 fiber knob, Ad41 short fiber knob, or Ad35 fiber knob. For example, mutations such as K01 and KO12, described in U.S. application Ser. No. 10/351,890 and incorporated herein by reference, are combined with mutations in the fiber shaft repeats such as those described herein, by PCR or other biochemical techniques known in the art. Combinations of the fiber knob mutations and the fiber shaft repeat modifications further reduce CAR interactions and provide detargeted adenoviral vectors.

One measurement of detargeting is the evaluation of the in vivo biodistribution of adenoviral vectors containing the modified fiber and their influence on adenoviral-mediated liver transduction. Examples of such assays are described in U.S. application Ser. No. 10/351,890. Cohorts of five C57BL/6 mice receive each vector via tail vein injection at a dose of 1×10¹³ particles per kg. The animals are sacrificed approximately 72 hours after vector administration and tissue samples such as liver, heart, lung, spleen, and kidney are collected from each animal.

Immunohistochemistry of tissues is used to assess tissue distribution of the virus. Staining with antibodies to viral proteins or to marker genes, such as β-galactosidase or GFP, are used to visualize positive cells. Additionally, enzymatic activity, fluorescence or other properties of genes expressed from the vectors are useful to monitor tissue distribution. Virus copy number is assessed in the different tissues, for example, by PCR analysis of hexon DNA. Detargeted viruses exhibit reductions in the number and/or intensity of hepatocytes that stain in the antibody assay or that exhibit marker gene activity as compared to assays with unmodified virus. Detargeting of tissues other than liver is assessed by similar methods and other methods known in the art.

E. Retargeting

The detargeted particles can be retargeted to selected tissues by adding binding specificity, such as by inclusion of a receptor ligand in the capsid.

1. Addition of Targeting Ligand

The viral particles that are detargeted as described herein, can be retargeted to selected cells and/or tissues by inclusion of an appropriate targeting ligand in the capsid. The ligand can be included in any of the capsid proteins, such as fiber, hexon and penton. Loci for inclusion of nucleic acid encoding a ligand is known to those of skill in the art for a variety of adenovirus serotypes; if necessary appropriate loci and other parameters can be empirically determined.

The ligand can be produced as a fusion by inclusion of the coding sequences in the nucleic acid encoding a capsid protein, or chemically conjugated, such as via ionic, covalent or other interactions, to the capsid or bound to the capsid (e.g., by antibody-ligand fusion, where the antibody binds capsid protein; or by disulfide bonding or other crosslinking moieties or chemistries).

Thus, for example, a modified fiber nucleic acid also can include sequences of nucleotides that encode a targeting ligand to produce viral particles that include a targeting ligand in the capsid. Targeting ligands and methods for including such ligands in viral capsids are well known. For example, inclusion of targeting ligands in fiber proteins is described in U.S. Pat. Nos. 5,543,328 and 5,756,086 and in U.S. application Ser. No. 09/870,203, published as U.S. Published application No. 20020137213, and International Patent Application No. PCT/EP01/06286. For different serotypes and strains of adenoviruses, loci for insertion of targeting ligands can be empirically determined. For different serotypes and strains, such loci can vary.

Because the adenovirus fiber has a trimeric structure, the ligand can be selected or designed to have a trimeric structure so that up to three molecules of the ligand are present for each mature fiber. Such ligands can be incorporated into the fiber protein using methods known in the art (see, e.g., U.S. Pat. No. 5,756,086). Instead of the fiber, the targeting ligand can be included in the penton or hexon proteins. Inclusion of targeting ligands in penton (see for example, in U.S. Pat. Nos. 5,731,190 and 5,965,431) and in hexon proteins (see for example, in U.S. Pat. No. 5,965,541) is known.

In one exemplary embodiment, the ligand is included in a fiber protein, which is a fiber protein mutated as described herein. As shown herein, the targeting ligand can be included, for example, within the Hi loop of the fiber protein. Any ligand that can fit in the HI loop and still provide a functional virus is contemplated herein. Such ligands can be as long as or longer than 80-100 amino acids (see, e.g., Belousova et al. (2002) J. Virol. 76:8621-8631). Such ligands are added by techniques known in the art (see, e.g., published International Patent Application publication No. WO99/39734 and U.S. application Ser. No. 09/482,682, published as US20030157688). Other ligands can be discovered through techniques known to those skilled in the art. Some non-limiting examples of these techniques include phage display libraries or by screening other types of libraries.

Targeting ligands include any chemical moiety that preferentially directs an adenoviral particle to a desired cell type and/or tissue. The categories of such ligands include, but are not limited to, peptides, polypeptides, single chain antibodies, and multimeric proteins. Specific ligands include the tumor necrosis factor (TNF) superfamily of ligands include, for example, TNFα and TNFβ, lymphotoxins (LT), such as LT-σ and LT-β, Fas ligand which binds to Fas antigen; CD40 ligand, which binds to the CD40 receptor of B-lymphocytes; CD30 ligand, which binds to the CD30 receptor of neoplastic cells of Hodgkin's lymphoma; CD27 ligand, NGF ligand, and OX-40 ligand; transferrin, which binds to the transferrin receptor located on tumor cells, activated T-cells, and neural tissue cells; ApoB, which binds to the LDL receptor of liver cells; alpha-2-macroglobulin, which binds to the LRP receptor of liver cells; alpha-I acid glycoprotein, which binds to the asialoglycoprotein receptor of liver; mannose-containing peptides, which bind to the mannose receptor of macrophages; sialyl-Lewis-X antigen-containing peptides, which bind to the ELAM-I receptor of activated endothelial cells; CD34 ligand, which binds to the CD34 receptor of hematopoietic progenitor cells; ICAM-I, which binds to the LFA-1 (CD11b/CD18) receptor of lymphocytes, or to the Mac-I (CD11a/CD18) receptor of macrophages; M-CSF, which binds to the c-fms receptor of spleen and bone marrow macrophages; circumsporozoite protein, which binds to hepatic Plasmodium falciparum receptor of liver cells; VLA-4, which binds to the VCAM-1 receptor of activated endothelial cells; HIV gp120 and Class II MHC antigen, which bind to the CD4 receptor of T-helper cells; the LDL receptor binding region of the apolipoprotein E (ApoE) molecule; colony stimulating factor, or CSF, which binds to the CSF receptor; insulin-like growth factors, such as IGF-I and IGF-II, which bind to the IGF-I and IGF-II receptors, respectively; Interleukins 1 through 14, which bind to the Interleukin 1 through 14 receptors, respectively; the Fv antigen-binding domain of an immunoglobulin; gelatinase (MMP) inhibitor; bombesin, gastrin-releasing peptide; substance P; somatostatin; luteinizing hormone releasing hormone (LHRH); vasoactive peptide (VIP); gastrin; melanocyte stimulating hormone (MSH); cyclic RGD peptide and any ligand or cell surface protein-binding (or targeting) molecule or molecule the targets particles with such modifications of selected cells or tissues.

2. Retargeting Achieved Through Modified Fibers

Ad particles are useful in gene therapy as vectors retargeted for specific cell types. One such example is the use of recombinant Ad vectors for gene therapy of diseases in which genes expressed in the photoreceptors are implicated. Such diseases include but are not limited to, degenerative ocular diseases, such as retinitis pigmentosa and Stargardt's disease. The tropism of Ad37 derives from the binding preference of its fiber protein, which binds to a receptor located on the surface of cells including Chang C, conjunctival epithelial cell line (Huang et al. (1999) J. Virology 73:2798-2802). Amino acids in the knob region of the Ad37 fiber have been implicated in the interaction between fiber and ocular cell surface receptors (Huang et al. (1999) J. Virology 73:2798-2802).

Ad vectors retargeted for ocular cells such as photoreceptor cells can be constructed. Chimeric fiber proteins containing the Ad37 fiber regions necessary for ocular and/or receptor cell binding, for example the Ad37 fiber knob, are combined with the fiber shaft modifications as described herein. Other fiber regions from adenoviruses with ocular tropism also can be used, such as other serotype D viruses,e.g. Ad8 and Ad19, including Ad19p. To further detarget the Ad vectors from non-ocular cells, additional fiber modifications can be added such as modifications of the HSP binding site as described herein.

Ocular targeting can be assessed by several methods. For example, Chang C cells are infected with Ad vectors expressing the modified fibers. These vectors are also constructed to express a marker gene such as GFP (such as described in the Examples). The cells are infected at 10,000 particles per cell, after incubation overnight cells are detached and washed and GFP fluorescence is measured. Adenovirus cell binding also can be measured (see U.S application Ser. No. 09/562,934; see, also corresponding published application US200020193327).

Retargeting to ocular cells such as photoreceptor cells with the vectors described herein also is assessed by producing virus particles with such vectors and injecting a solution containing approximately 1×10⁹ particles/μl into the vitreous chamber of a mouse eye. Seven days post-injection, eyes are harvested, fixed with paraformaldehyde and cryo-sectioned. Sections are stained with an anti-rhodopsin antibody to identify photoreceptor cells and with DAPI to show all cell nuclei. GFP staining indicates transduced cells (see U.S. application Ser. No. 09/562,934; see, also corresponding published application US200020193327).

Ad vectors retargeted to ocular cells as described herein are also useful in the therapy of retinal disorders, such as retinal blastomas. Therapeutic agents can be encoded by these recombinant adenoviral vectors include, but are not limited to, trophic factors, such as glial cell line-derived neurotrophic factor (GDNF) and ciliary neurotrophic factor (CNTF), growth factors and growth factor inhibitors, anti-apoptotic factors, such as Bcl-2 (CNTF), anti-tumor agents, anti-angiogenics, and genes or portions thereof for gene replacement or repair of defective genes.

Adenoviral vectors are also useful in gene therapy when retargeted to dendritic cells. Dendritic cells, which have a variety of important physiological features in the immune system, can serve as targets for immunotherapy and vaccine development. Dendritic cells pick up antigens and migrate from the tissues of the body to the lymphoid tissues. There these cells present the antigens in the lymphoid organs by displaying a foreign epitope bound to an MHC protein and trigger humoral and cellular immune responses. Such antigen-presenting cells (APCs) are part of the immune response mechanism. Genetically modified dendritic cells that express particular antigens, such as tumor antigens, can be used as vaccines. Numerous studies have shown that adenovirus (Ad)-mediated delivery to dendritic cells dendritic cells can lead to anti-tumor response. These vectors can deliver heterologous nucleic acids to alter dendritic cell antigen presentation, cytokine production and other dendritic cell functions.

Fibers from certain non CAR-using Ad serotypes bind to receptors on dendritic cells. Particularly effective are fibers, or portions thereof, from subgroup D such as the Ad19p, Ad37 or Ad16 (see, e.g., U.S. Provisional Application No. 60/467,500). Ad vectors retargeted for dendritic cells can be constructed using the modified fibers as described herein, reduced for CAR binding, combined with fiber portions that redirect the recombinant vectors to dendritic cells such as the modifications described in the provisional application, for example combinations with fiber portions containing fiber knobs or portion thereof from a serotype D Ad fiber.

Dendritic targeting can be assessed in vitro for example by generating bone marrow-derived dendritic cells by culture of bone marrow cells from female Balb/C mice and using cell surface markers such as staining with fluorescently-conjugated antibodies directed against CD11c, CD80, and CD86 for confirmation. Primary dendritic cell cultures are infected with 100,000 viral particles/cell of Ad5.GFP.ΔF pseudotyped with the modified fibers. GFP expression is used to monitor cell infectivity.

F. Delivery of Heterologous Products

Adenovirus particles can be used to express heterologous nucleic acids, such as for delivery of a gene product to a targeted cell.

1. Heterologous Polypeptides

The packaged adenoviral genome also can contain a heterologous polynucleotide that encodes a product of interest, such as a therapeutic protein. Adenoviral genomes containing heterologous polynucleotides are well known (see, e.g., U.S. Pat. Nos. 5,998,205, 6,156,497, 5,935,935, and 5,801,029). These can be used for in vitro, ex vivo and in vivo delivery of the products of heterologous polynucleotides or the heterologous polynucleotides.

Thus, the adenoviral particles provided herein can be used to engineer a cell to express a protein that it otherwise does not express or does not express in sufficient quantities. This genetic engineering is accomplished by infecting the desired cell with an adenoviral particle whose genome includes a desired heterologous polynucleotide. The heterologous polynucleotide is then expressed in the genetically engineered cells. For use herein, the cell is generally a mammalian cell, and is typically a primate cell, including a human cell. The cell can be inside the body of the animal (in vivo) or outside the body (in vitro). Heterologous polynucleotides (also referred to as heterologous nucleic acid sequences) are included in the adenoviral genome within the particle and are added to that genome by techniques known in the art. Any heterologous polynucleotide of interest can be added, such as those disclosed in U.S. Pat. No. 5,998,205, incorporated herein by reference.

Polynucleotides that are introduced into an Ad genome or vector can be any that encode a protein of interest or that are regulatory sequences. Proteins include, but are not limited to, therapeutic proteins, such as an immunostimulating protein, such as an interleukin, interferon, or colony stimulating factor, such as granulocyte macrophage colony stimulating factor (GM-CSF; see, e.g., U.S. Pat. No. 5,908,763. Generally, such GM-CSF is a primate GM-CSF, including human GM-CSF. Other immuno-stimulatory genes include, but are not limited to, genes that encode cytokines IL1, IL2, 1L4, IL5, IFN, IFN, TNF, IL12, IL18, and flt3), proteins that stimulate interactions with immune cells (B7, CD28, MHC class I, MHC class II, TAPs), tumor-associated antigens (immunogenic sequences from MART-1, gp100(pmel-17), tyrosinase, tyrosinase-related protein 1, tyrosinase-related protein 2, melanocyte-stimulating hormone receptor, MAGE1, MAGE2, MAGE3, MAGE12, BAGE, GAGE, NY-ESO-1, -catenin, MUM-1, CDK-4, caspase 8, KIA 0205, HLA-A2R1701, α-fetoprotein, telomerase catalytic protein, G-250, MUC-1, carcinoembryonic protein, p53, Her2/neu, triosephosphate isomerase, CDC-27, LDLR-FUT, telomerase reverse transcriptase, and PSMA), cDNAs of antibodies that block inhibitory signals (CTLA4 blockade), chemokines (MIP1, MIP3, CCR7 ligand, and calreticulin), and other proteins.

Other polynucleotides, including therapeutic nucleic acids, such as therapeutic genes, of interest include, but are not limited to, anti-angio-genic, and suicide genes. Anti-angiogenic genes include, but are not limited to, genes that encode METH-1, METH-2, TrpRS fragments, pro-liferin-related protein, prolactin fragment, PEDF, vasostatin, various fragments of extracellular matrix proteins and growth factor/cytokine inhibitors. Various fragments of extracellular matrix proteins include, but are not limited to, angiostatin, endostatin, kininostatin, fibrinogen-E fragment, thrombospondin, tumstatin, canstatin, and restin. Growth factor/cytokine inhibitors include, but are not limited to, VEGF/VEGFR antagonist, sFIt-1, sFIk, sNRP1, angiopoietin/tie antagonist, sTie-2, chemokines (IP-10, PF-4, Gro-beta, IFN-gamma (Mig), IFN, FGF/FGFR antagonist (sFGFR), Ephrin/Eph antagonist (sEphB4 and sephrinB2), PDGF, TGF and IGF-1.

Among therapeutic transgenes that can be included in the viral constructs and resulting particles are those that result in an “armed” virus. For example, rather than delete E3 region as in some embodiments described herein, all or a part of the E3 region can be preserved or re-inserted in an oncolytic adenoviral vector (discussed above). The presence of all or a part of the E3 region can decrease the immunogenicity of the adenoviral vector. It also increases cytopathic effect in tumor cells and decreases toxicity to normal cells. Typically such vector expresses more than half of the E3 proteins.

A “suicide gene” encodes a protein that can lead to cell death, as with expression of diphtheria toxin A, or the expression of the protein can render cells selectively sensitive to certain drugs, e.g., expression of the Herpes simplex thymidine kinase gene (HSV-TK) renders cells sensitive to antiviral compounds, such as acyclovir, gancyclovir and FIAU (1-(2-deoxy-2-fluoro-1-beta-D-arabinofuranosil)-5-iodouracil). Other suicide genes include, but are not limited to, genes that encode carboxypeptidase G2 (CPG2), carboxylesterase (CA), cytosine deaminase (CD), cytochrome P450 (cyt-450), deoxycytidine kinase (dCK), nitroreductase (NR), purine nucleoside phosphorylase (PNP), thymidine phosphorylase (TP), varicella zoster virus thymidine kinase (VZV-TK), and xanthine-guanine phosphoribosyl transferase (XGPRT). Alternatively, a therapeutic nucleic acid can exert its effect at the level of RNA, for instance, by encoding an antisense message or ribozyme, a protein that affects splicing or 3′ processing (e.g., polyadenylation), or a protein that affects the level of expression of another gene within the cell, e.g. by mediating an altered rate of mRNA accumulation, an alteration of mRNA transport, and/or a change in post-transcriptional regulation.

The addition of a therapeutic nucleic acid to a virus results in a virus with an additional antitumor mechanism of action. Thus, a single entity (i.e., the virus carrying a therapeutic transgene) is capable of inducing multiple antitumor mechanisms. Other encoded proteins, include, but are not limited to, herpes simplex virus thymidine kinase (HSV-TK), which is useful as a safety switch (see, U.S. patent application Ser. No. 08/974,391, filed Nov. 19, 1997, which published as PCT Publication No. WO/9925860), Nos, FasL, and sFasR (soluble Fas receptor).

Other products for delivery to cells, such as immune cells, including dendritic, are tumor antigens. Tumor antigens include, but are not limited to carcinoembryonic antigen, NY-BR1, NY-ESO-1, MAGE-1, MAGE-3, BAGE, GAGE, SCP-1, SSX-1, SSX-2, SSX-4, CT-7, Her2/Neu, NY-BR-62, NY-BR-85 and tumor protein D52 (Scanlan and Jäger (2001) Breast Cancer Res. 3:95-98; Yu and Restifo (2002) J. Clin. Invest. 110:289-94). The following Table includes an exemplary list of tumor antigens and tissues expressing such antigens.

| Exemplary Antigens | Tumor Tissue |
|---|---|
| Oncofetal | |
| OPA | Fetal pancreas |
| CEA | Colon, Rectal, Stomach, Lung, Pancreas, |
| | Kidney, Bladder, Head & Neck, Cervical, |
| | endometrial, ovarian, Breast |
| POA | Fetal pancreas |
| FAP | Fetal pancreas |
| PA8-15 | Pancreatic cancer cell line SUIT-2 |
| Adult | |
| CA 50 | Colorectal carcinoma cell line |
| CA 19-9 | Colon carcinoma cell line SW1116 |
| CA 242 | Colorectal carcinoma cell line COLO 205 |
| CAR-3 | Epidermoid carcinoma cell line A 431 |
| DU-PAN-2 | Pancreatic carcinoma cell line HPAF |
| Ypan-1 | Pancreatic carcinoma cell line SW1990 |
| Span-1 | Pancreatic carcinoma cell line SW1990 |
| BW494 | Pancreatic tumor tissue |
| MUSE 11 | Gastric cancer ascites fluid |
| L_{A1} | Embryonal carcinoma cells |
| Le^{a} | Colon adenocarcinoma |
| Fuc-L_{A1} | Pancreatic adenocarcinoma |
| Le^{b} | Colon adenocarcinoma |
| | Pancreatic adenocarcinoma |
| 3-isoL_{M1} | Small cell lung carcinoma |
| | Glioma |
| | Medulloblastoma |
| | Teratocarcinoma cells |
| 3′,6′-isoL_{D1} | Liver metastasis of colon cancer |
| | Embryonal carcinoma cells |
| Fuc-3′-isoL_{M1} | Gastrointestinal cancer |
| Sialylated Le^{a} | |
| Fuc-3′,6′-isoL_{D1} | Human colon adenocarcinoma |
| Disialylated Le^{a} | |
| nL_{A1} | Colon cancer |
| i-Antigen | Lung cancer |
| SSEA-1 | Teratocarcinoma |
| Le^{x} | Colon cancer |
| Fuc-nL_{A1} | |
| Dimeric Le^{x} | Adenocarcinoma |
| | Colon cancer |
| | Liver cancer |
| Le^{y} | Gastric cancer |
| | Breast cancer |
| | Colon cancer |
| 6′-L_{M1} | Colorectal carcinoma |
| | Lung carcinomas |
| | Primary hepatoma |
| Sialylated Le^{x} or | Gastrointestinal cancer |
| Fuc-3′-L_{M1} | Lung carcinoma |
| | Gastric |
| | colon |
| | lung |
| | breast |
| | renal cancers |
| GB3 | Burkitt′s lymphoma |
| Globo-H | breast cancer |
| Sulfatide | Mucinous cystadenocarcinoma, |
| Disulfated G_{A1} | Hepatocellular carcinoma |
| N-Glycolylneuraminic | Colon cancer |
| acid | |
| N-Glycolyl-G_{M2} | N-Glycolyl-G_{M2} |
| G_{M2} | Melanoma |
| OFA-I-1 | |
| OFA-I-2 | |
| | Glioma |
| | Germ cell tumors |
| G_{D2} | Melanoma |
| | Neuroblastoma |
| | Small cell lung carcinoma |
| | Glioma |
| Gₘ₃ | Melanoma |
| Ag | |
| FCM1 | |
| 2-39 | |
| IF43 | |
| gp-100 melanoma- | |
| associated antigen | |
| G_{D3} | Melanoma |
| HJM1 | Melanoma |
| | Medulloblastoma |
| | Glioma |
| | Leukemia |
| | Meningioma |
| 9-O-Acetyl-G_{D3} | Melanoma |
| Fuc-G_{M1} | Small cell lung carcinoma |
| COTA | Colon, ovarian |
| SW1038 | Colon |
| CTS | prostate |
| MAGE-1 MAGE-2 MAGE-3 | Lung |
| (MZ2-E MZ2-Bb) | melanocyte |
| | breast |
| MUC-1 | Breast |
| | pancreas |
| Lewis-Ag (GICA) | Ovarian |
| | myelin |
| TAG-12 | Breast |
| | ovarian |
| TAG-72 | colon |
| | ovarian pancreas |
| Organ-specific cancer | Lung |
| neoantigen (OSN) | |
| GP100 | Melanocyte |
| MART-1 | Melanocyte |
| p95/p97 | Melanocyte |
| EGF receptor | Squamous tumors |
| CA125 | Ovary |
| | Breast |
| _{P}97 (melanotransferrin) | Melanocyte |
| 22-1-1 | uterus |
| | cervix |
| | ovary |
| GA733 | gastrointestinal carcinoma |
| YH206 | adenocarcinomas |
| MART-2 | melanocytes |
| BAGE-1 | melanocytes |
| GAGE1-6 | melanocyte |
| | osteosarcoma |
| DF3 | Breast |
| | lymphocytes |
| L3p40-50 | Lung |
| L3p90 | |
| Thomsen-Friedenrich | pancarcinoma |
| Pan Tumor Antigen | |
| | pancreas |
| | ovarian |
| EPB-2 | B cell lymphoma |
| | melanoma |
| | lymphoma |
| | medullary thyroid carcinoma |
| | gastrointestinal carcinoma |
| NS-ESO-1 | melanoma, breast, bladder, prostate, |
| | hepatocellular carcinoma |
| NY-ESO-1 | melanoma, breast, bladder, prostate, |
| | hepatocellular carcinoma |

Also contemplated are combinations of two or more heterologous proteins that can exhibit synergistic, complementary and/or nonoverlapping toxicities and methods of action. The resulting adenovirus can retain the viral oncolytic functions and, for example, additionally is endowed with the ability to induce immune and anti-angiogenic responses and other responses as desired.

2. Gene Expression and Regulation

a. Heterologous polynucleotides

Therapeutic polynucleotides and heterologous polynucleotides also include those that exert an effect at the level of RNA or protein. These include a factor capable of initiating apoptosis, RNA, such as RNAi and other double-stranded RNA, antisense and ribozymes, which among other capabilities can be directed to mRNAs encoding proteins essential for proliferation, such as structural proteins, transcription factors, polymerases, genes encoding cytotoxic proteins, genes that encode an engineered cytoplasmic variant of a nuclease (e.g. Rnase A) or protease (e.g. trypsin, papain, proteinase K and carboxypeptidase). Other polynucleotides include a cell or tissue specific promoters, such as those used in oncolytic adenoviruses (see, e.g., U.S. Pat. No. 5,998,205).

b. Regulation of Gene Expression

As noted, the adenovirus vectors also can include heterologous nucleic acids that encode or provide products, such as therapeutic products or that alter gene expression. Any therapeutic product is contemplated and a variety are set forth herein as exemplary. Heterologous nucleic acid can encode a polypeptide or include or encode a regulatory sequence, such as a promoter or an RNA. The heterologous nucleic acid can encode small RNAs, including RNAi, other double-stranded RNA (dsRNA), antisense RNA, and ribozymes, that can alter gene expression. Promoters include, for example, constitutive and regulated promoters and tissue specific promoters, including tumor specific promoters. The promoter can be operably linked, for example, to a gene of an adenovirus essential for replication.

The heterologous polynucleotide encoding a polypeptide also can contain a promoter operably linked to the coding region. One example is a regulated promoter and transcription factor expression system, such as the published tetracycline-regulated systems, or other regulatable systems (see for example, WO 01/30843), to allow regulated expression of the encoded polypeptide. An exemplary regulatable promoter system is the Tet-On and Tet-Off system (e.g., available from Clontech (Palo Alto, Calif.)). This promoter system allows the regulated expression of the transgene controlled by tetracycline or tetracycline derivatives, such as doxycycline. This system can be used to control the expression of the encoded polypeptide in the viral particles and nucleic acids provided herein. Other regulatable promoter systems are known (see, e.g., U.S. Published Application No. 20020168714). Regulatable promoters also include tissue-specific promoters. Tissue-specific promoters direct the expression of the gene to which they are operably linked to a specific cell type. Tissue-specific promoters cause the gene located 3′ of it to be expressed predominantly, if not exclusively, in the specific cells where the promoter expressed its endogenous gene. Typically, it appears that if a tissue-specific promoter expresses the gene located 3′ of it at all, then it is expressed appropriately in the correct cell types (see, e.g., Palmiter et al. (1986) Ann. Rev. Genet. 20: 465-499). Tissue-specific promoters useful in Ad vectors such as those described herein are for example tumor-specific promoters, such as those used in oncolytic adenoviruses (see, e.g., U.S. Pat. No. 5,998,205), ocular cell-specific promoters, such as the rhodopsin promoter, and dendritic cell-specific promoters, and tissue-selective promoters such as those described in U.S. Pat. No. 5,998,205. Other suitable promoters that can be employed include, but are not limited to, adenoviral promoters, such as the adenoviral major late promoter and/or the E3 promoter; or heterologous promoters, such as the cytomegalovirus (CMV) promoter; the Rous Sarcoma Virus (RSV) promoter; inducible promoters, such as the MMT promoter, the metallothionein promoter; heat shock promoters; the albumin promoter; and the ApoAI promoter.

The heterologous polynucleotide also can include an adenovirus tripartite leader (TPL) nucleic acid sequence (for example SEQ ID No. 22) operatively linked to an intron containing RNA processing signals (such as for example, splice donor or splice acceptor sites) suitable for expression in the packaging cell line. Generally the intron contains a splice donor site and a splice acceptor site. Alternatively, the TPL nucleotide sequence does not contain an intron. The intron includes any sequence of nucleotides that function in the packaging cell line to provide RNA processing signals, including splicing signals. Introns have been well not limited to a native intron 1 from adenovirus, such as Ad5's TPL intron 1; others include the SV40 VP intron; the rabbit beta-globin intron, and synthetic intron constructs (see, e.g., Petitclerc et al. (1995) J. Biothechhol, 40:169; and Choi et al. (1991) Mol. Cell. Biol., 11:3070).

The nucleic acid molecule encoding the TPL can include, for example, the native TPL with at least the for first intron or, for example, either (a) first and second TPL exons or (b) first, second and third TPL exons, where each TPL exon in the sequence is selected from among the complete TPL exon 1, partial TPL exon 1, complete TPL exon 2 and complete TPL exon 3. A complete exon is one that contains the complete nucleic acid sequence based on the sequence found in the wild type viral genome. The TPL exons typically are from Ad2, Ad3, Ad5 and Ad7; they can be derived from any Ad serotype, as described herein. For example, a TPL with a partial exon 1 can be used (see, e.g., International PCT application No. WO 98113499 and U.S. application Ser. No. 09/795,292, published as US20040002060).

G. Animal and Human Delivery

The adenoviral vectors provided herein can be used to study cell transduction and gene expression in vitro or in various animal models. The latter case includes ex vivo techniques, in which cells are transduced in vitro and then administered to the animal. Ad vectors provided herein also can be used to conduct gene therapy on humans or other animals. Such gene therapy can be ex vivo or in vivo. For in vivo gene therapy, the adenoviral particles in a pharmaceutically-acceptable carrier are delivered to a human in a therapeutically effective amount in order to prevent, treat, or ameliorate a disease or other medical condition in the human through the introduction of a heterologous gene that encodes a therapeutic protein into cells in such humans. The adenoviruses are delivered at a dose ranging from approximately 1 particle per kilogram of body weight to approximately 10¹⁴ particles per kilogram of body weight. Generally, they are delivered at a dose of approximately 10⁶ particles per kilogram of body weight to approximately 10¹³ particles per kilogram of body weight, and typically the dose ranges from approximately 10⁸ particles per kilogram of body weight to approximately 10¹² particles per kilogram of body weight.

Gene therapy methods include in vivo and ex vivo methods. In all methods involving expressing heterologous nucleic acids, vectors containing the nucleic acids are transduced into a cell or cells. In these methods an adenoviral vector provided herein is transduced into a cell to deliver the nucleic acid and/or encoded products. Transduction can be effected in vivo or in vitro or ex vivo, and can be for a variety of purposes including study of gene expression and genetic therapy. The cells can be prokaryotic cells, but typically are eukaryotic cells, including mammalian cells, such as primate, including human, cells. The cells can be of a specific type, such as a tumor cell or a cell in a particular tissue. The vectors can be oncolytic vectors to effect killing of tumor cells.

Propagation and Scale-Up

Since doubly ablated adenoviral vectors containing mutations in the fiber and/or penton capsid proteins can result in inefficient cell binding and entry via the CAR/αv integrin entry pathway, scaled up technologies improve the growth and propagation of such vectors to produce high titers of the adenoviral vectors for clinical use. Multiple strategies can be used to scale up vectors that are detargeted via fiber and/or penton modifications. These include: (a) the use of pseudoreceptor cell lines engineered to express a surface receptor that binds a ligand displayed on the vector (see, e.g., International PCT application No. WO 98/54346) and (b) complementing cell lines that are engineered to express native fiber and that can be engineered to express native fiber and penton (see, e.g., International PCT application No. WO 00/42208; (c) the use of polycations and/or bifunctional reagents, which when added to tissue culture medium, bind adenoviral particles and direct their entry into the producer cells; and (d) other strategies known to those of skill in the art. In this latter method (see, copending U.S. application Ser. No. 10/351,890 and International PCT application No. PCT/US03/02295), reagents (also called medium additives) also can be included in the tissue culture medium containing producer cells to be infected with the detargeted adenoviral vectors. Alternatively the reagents can be pre-mixed with the virus, which mixture is then added to the tissue producer cells. Reagents that are useful in this method are those that are capable of directing adenoviral particle entry into the producer cells. Such reagents include, but are not limited to, polycations and bifunctional reagents. Examples of suitable reagents are those described in patent application Ser. No. 10/351,890 and International PCT application No. PCT/US03/02295, such as polytheylenimine, protamine sulfate, poly-L-lysine, hexadimethrine bromide and bifunctional reagents such as anti-fiber antibody ligand fusions, anti-fiber-Fab-FGF conjugate, anti-penton-antibody ligand fusions, anti-hexon antibody ligand fusions and polylysine-peptide fusions.

H. Formulation and Administration

Compositions containing therapeutically effective concentrations of the recombinant adenovirus particles are provided. The particles are formulated in any suitable vehicle, such as by mixing, and at a suitable concentration, including concentrated formulations for dilution and single dosage formulations. Administration is effected by any means, including systemic administration, such as intramuscular, parenteral and intravenous administration, local or topical administration depending upon the treatment. The compositions can be formulated in sustained released formulations, in liposomes and in other delivery vehicles. Sustained release formulations can be formulated for multiple dosage administration, so that during a selected period of time, such as a month or up to about a year, several dosages are administered. Thus, for example, liposomes can be prepared such that a total of about two to up to about five or more times the single dosage is administered in a single administration.

To prepare compositions the viral particles are dialyzed into a suitable acceptable carrier or viral particles, for example, can be concentrated and/or mixed therewith. The resulting mixture can be a solution, suspension or emulsion. In addition, the viral particles can be formulated as the sole pharmaceutically active ingredient in a composition or can be combined with other active agents for the particular disorder treated.

The following examples are included for illustrative purposes only and are not intended to limit the scope of the invention.

### EXAMPLE 1

### Construction of Chimeric Fiber Proteins

Chimeric adenovirus fiber proteins were constructed using gene splicing by overlap extension PCR (Horton et al., (1990) J. Virol. 74:10274-10286) from Ad5 and Ad37 fiber gene fragments. PCR and mutagenic primers are listed in Table 2.

TABLE 2
PCR, overlap extension PCR, and mutagenic primers.

| | | | |
|---|---|---|---|
| Construct | Primer | Sequence | SEQ ID |

| | | | |
|---|---|---|---|
| Ad37s/Ad5k | L37 | 5′-TGT CTT GAA TCC AAG ATG* AND AAG CGC | 1 |
| | | GCC CGC CCC AGC GAA GAT GAC TTC-3′ | |
| | 37s5k-3 | 5′-TGG AGC TGG TGT GGT CCA CAA AGT GCG | 2 |
| | | CGT GTC ATA TTC TGG GTT CCA-3′ | |
| | 5k-5 | 5′-ACT TTG TGG ACC ACA CCA GCT CCA-3′ | 3 |
| | fiber3 | 5′-CAT AAC GCG GCC GCT TCT TTA TTC TTG | 4 |
| | | GGC-3′ | |
| | M-c1-f | 5′-GTG CTA CTA AAC AAT TCC TTC CTG GAT | 5 |
| | | CCA GAA TAT TGG AAC-3′ | |
| | M-c1-r | 5′-GTT CCA ATA TTC TGG ATC CAG GAA GGA | 6 |
| | | ATT GTT TAG TAG CAC-3′ | |
| Ad5s/Ad37k | fiber5 | 5′-ATG GGA TCC AAG ATG* AAG CGC GCA | 7 |
| | | AGA CCG-3′ | |
| | 5s-3 | 5′-TGG TGT GGT CCA CAA AGT TAG CTT ATC | 8 |
| | | ATT-3′ | |
| | 5s37k-5 | 5′-AAG CTA ACT TTG TGG ACC ACA CCA GAC | 9 |
| | | ACA TCT CCA AAC TGC ACA ATT-3′ | |
| | 37fr | 5′-AAA CAC GGC GGC CGC TCT TTC ATT CTT | 10 |
| | | G-3′ | |
| | M-c2-f | 5′-CTT TGT GGA CCA CAC CAG ACA CTA GTC | 11 |
| | | CAA ACT GCA CAA TTG CTC-3′ | |
| | M-c2-r | 5′-GAG CAA TTG TGC AGT TTG GAC TAG TGT | 12 |
| | | CTG GTG TGG TCC ACA AAG-3′ | |
| Ad5Δs | short3 | 5′-GCT TAG GTT AAC CTC AAG CTT TTT CTT | 13 |
| | | GGT TTT TTT GAG AGG TGG GCT-3′ | |
| | short5 | 5′-AGC CCA CCT CTC AAA AAA ACC AGG AAA | 14 |
| | | AAG CTT GAG GTT AAC CTA AGC-3′ | |
| Ad5s/Ad37s | rep-3 | 5′-ATC AGT ATT AAC TTG CAG TGG AGC CTT | 15 |
| | | AGG GTT TAC AGT TAG GCT TCC GGC CTC GTC | |
| | | CAG AGA GAG GCC GTT-3′ | |
| | rep3-5 | 5′-GGA AGC CTA ACT GTA AAC CCT AAG GCT | 16 |
| | | CCA CTG CAA GTT AAT ACT GAT TCA AAC | |
| | | ATA AAC CTG GAA ATA TCT-3′ | |
| | rep7-3 | 5′-ATC ATT GTC AAA TGT CAA CCC TTC TCT | 17 |
| | | TGC TCT TAC ATT TAT ACC AAT GTT GTA ATC | |
| | | AAA TTC TAG GCC ATG-3′ | |
| | rep7-5 | 5′-ATT GGT ATA AAT GTA AGA GCA AGA GAA | 18 |
| | | GGG TTG ACA TTT GAC AAT GAT GGT GCC ATT | |
| | | ACA GTA GGA AAC AAA-3′ | |
| | Mut4for | 5′-CTG GAC GAG GCC GGC AGC CTA ACT GTA | 19 |
| | | AAC CCT AAG GC-3′ | |
| | Mut4rev | 5′-GGC TTA GGG TTT ACA GTT AGG CTG CCG | 20 |
| | | GCC TCG TCC AG-3′ | |

Introduced restriction sites are in bold.
The start codons are denoted by an asterisk (*).
Mutagenic bases are underlined.

Ad37s/Ad5k: Approximately 10⁸ particles of wild-type Ad37 (ATCC) were mixed with a PCR master mix (1× ThermoPol Buffer, 300 μM each dATP, dTTP, dGTP, and dCTP, and 2 U Vent DNA polymerase, New England Biolabs, Beverly, Mass.), 200 nM primers L37 (SEQ ID No. 1) and 37s5k-3 (SEQ ID No. 2), to amplify the nucleotide sequence encoding amino acids 1-184 of the Ad37 fiber. Mutations were incorporated into the Ad37 fiber tail to make the sequence more closely match the Ad5 tail (Wu et al., (2001) Virology 279: 78-89). These mutations change the first seven amino acids of the tail from MSKRLRV of Ad37 (SEQ ID No. 37) to MKRARPS of Ad5 (SEQ ID No. 35) to facilitate fiber incorporation into the Ad5 vector capsid. This PCR reaction mixture was heated to 94° C. for 5 minutes and subjected to 1 cycle of 94° C. for 1 minute, 45° C. for 1.5 minutes, and 72° C. for 2 minutes, then 30 cycles of 94° C. for 1 minute, 50° C. for 1.5 minutes, and 72° C. for 2 minutes, and a final extension step of 72° C. for 5 minutes (Program 1).

To construct the Ad5 portions of the chimeras, pDV67 (available from the ATCC under accession number PTA-1145) was used as a starting material. The nucleotide sequence of pDV67 is set forth in SEQ ID No. 21. pDV67 has the TPL cassette and the Ad5 fiber gene inserted into a pcDNA3.1/Zeo(+) backbone (see U.S. Application Ser. No. 60/459,000; see also, Von Seggern et al. (1998) J. Gen Virol., 79: 1461-1468).

In a second reaction, pDV67 was mixed with PCR master mix. Primers 5k-5 (SEQ ID No. 3) and fiber3 (SEQ ID No. 4) were added to this reaction to amplify the nucleotide sequence encoding amino acids 400-581 of the Ad5 fiber. This PCR reaction mixture was heated to 94° C. for 5 minutes and subjected to 30 cycles of 94° C. for 1 minute, 55° C. for 1 minute, and 72° C. for 2 minutes, and a final extension step of 72° C. for 5 minutes (Program 2).

The first-step PCR products were gel purified from 10 μl of the 100 μl reactions in a 1% low melting agarose gel. The gel purified PCR products were melted and 10 μl of each were mixed together with 1× PCR Buffer, an additional 3 mM MgCl₂, 300 μM each dNTP, 0.8 μM L37 5′ primer (SEQ ID No. 1) and fiber3 3′ primer (SEQ ID No. 4), and 5 U Taq DNA polymerase (Gibco BRL; Invitrogen, Carlsbad, Calif.). Program 1, as described above, was used for the overlap extension PCR reaction.

The PCR product was cloned into the pCR2.1 cloning vector (SEQ ID No. 69) using the TOPO TA Cloning Kit (Invitrogen, Carlsbad, Calif.). The plasmid was transformed into TOP10 E. coli cells (Invitrogen) and purified from cultured cells using the Qiagen Plasmid Mini Spin Kit (Qiagen, Valencia, Calif.). The chimeric fiber gene was excised from pCR2.1 and ligated into the BamHI and NotI sites of pcDNA3.1zeo(+) (Invitrogen). The Ad5 tripartite leader (TPL; SEQ ID No. 22) was excised from pDV55 using BamHI and BglII and inserted into the BamHI site in front of the chimeric fiber gene in the expression vector (construction of plasmid pDV55 is described in copending U.S. application Ser. No. 09/482,682 published as US20030157688, see, also International PCT application No. PCT/US00/00265; and in U.S. application Ser. No. 09/562,934 (see, also corresponding published application US200020193327), also filed as International PCT application No. PCT/EP01/04863, each incorporated by reference herein). The plasmid containing Ad37s/Ad5k was designated pLP13.

Vectors containing Ad5s/Ad37k, Ad5Δs, and Ad5s/Ad37s genes preceded by the Ad5 TPL, designated pLP23, pLP32, and pLP43, respectively, were constructed in the same fashion as Ad37s/Ad5k, using the primers, templates, and PCR programs listed in Table 3. The chimeric fiber proteins Ad37s/Ad5k, Ad5s/Ad37k, Ad5Δs, and Ad5s/Ad37s are shown schematically in FIG. 2.

All four plasmids, pLP13, pLP23, pLP32, and pLP43, were purified from 500 ml cultures using the Qiagen Plasmid Maxi Kit.

TABLE 3
Overlap extension polymerase chain reactions for construction of chimeric fiber proteins.

| | | | | | | | Amplified fiber |
|---|---|---|---|---|---|---|---|
| Rxn | Construct | Template | Polymerase | 5′ Primer | 3′ Primer | Program | protein fragment |
| 1 | Ad37s/Ad5k | wt Ad37 virus | Vent* | L37 | 37s5k-3 | 1 | Ad37 1-184 |
| 2 | ″ | pDV67 | Vent | 5k-5 | fiber3 | 2 | Ad5 400-581 |
| 3 | ″ | Rxns. 1 | Taq† | L37 | fiber3 | 1 | Ad37 1-184; |
| 4 | Ad5s/Ad37k | pDV67 | Vent | fiber5 | 5s-3 | 2 | Ad5 1-405 |
| 5 | ″ | wt Ad37 virus | Vent | 5s37k-5 | 37fr | 2 | Ad37 188-365 |
| 6 | ″ | Rxns. 4 | Taq | fiber5 | 37fr | 1 | Ad5 1-405; |
| 7 | Ad5Δs | pDV67 | Vent | fiber5 | short3 | 2 | Ad5 1-94 |
| 8 | ″ | pDV67 | Vent | short5 | fiber3 | 2 | Ad5 317-581 |
| 9 | ″ | Rxns. 7 | Taq | fiber5 | fiber3 | 1 | Ad5 1-94 and |
| 10 | Ad5s/Ad37s | pDV67 | Vent | fiber5 | rep3-3 | 2 | Ad5 1-75; |
| | | | | | | | Ad37 74-89 |
| 11 | ″ | pDV67 | Vent | rep3-5 | rep7-3 | 2 | Ad5 95-370; |
| | | | | | | | Ad37 74-89 |
| | | | | | | | and 166-171 |
| 12 | ″ | pDV67 | Vent | rep7-5 | fiber3 | 2 | Ad5 387-581; |
| | | | | | | | Ad37 166-171 |
| 13 | ″ | Rxns. 11 & 12 | Taq | rep3-5 | fiber3 | 2 | Ad5 95-370 |
| | | | | | | | and 387-581; |
| | | | | | | | Ad37 74-89 |
| | | | | | | | and 166-171 |
| 14 | ″ | Rxns. 10 & 13 | Taq | fiber5 | fiber3 | 1 | Ad5 1-75, |
| | | | | | | | 95-370 and |
| | | | | | | | 387-581; |
| | | | | | | | Ad37 74-89 |
| | | | | | | | and 166-171 |

*Vent DNA Polymerase; New England Biolabs.
†Taq DNA Polymerase; Gibco BRL

Construction of pDV121

To construct a plasmid for the expression of the Ad37 fiber, the open reading frame was PCR amplified from viral genomic DNA of Ad37 using primers L37 (SEQ ID No. 1) and 37fr (SEQ ID No. 10) and cloned into pCR2.1 TOPO (Invitrogen, Carlsbad, Calif.; see, SEQ ID No. 70) to create pDV117. After confirmation of the correct sequence, the Ad37 fiber open reading frame was excised from pDV117 using BamHI and NotI sites contained in the PCR primer, and inserted into the BamHI and NotI sites of pcDNA3.1zeo(+)(Invitrogen) to create pDV120. The BamHI-BgIII fragment was excised from pDV55, as described above, and inserted into the BamHI site of pDV120 to create the plasmid pDV121.

### EXAMPLE 2

### Construction of Fiber-Expressing packaging Cell Lines

Cell Lines

HEK-293T cells (DuBridge, et al, (1987) Mol. Cell. Biol. 7:379-387) and A549 lung carcinoma cells (American Type Culture Collection, Manassas, Va.) were cultured in Dulbecco's Modified Eagle Medium (Gibco BRL; Invitrogen, Carlsbad, Calif.) containing 10% fetal bovine serum (FBS; Mediatech, Herndon, Va.).

AE1-2a S8 cells are derivatives of the A549 lung carcinoma line (ATCC no. CCL 185) with chromosomal insertions of the plasmids pGRE5-2.E1 (also referred to as GRE5-E1-SV40-Hygro construct and set forth in SEQ ID No. 23) and pMNeoE2a-3.1 (also referred to as MMTV-E2a-SV40-Neo construct and set forth in SEQ ID No. 24), which provide complementation of the adenoviral E1 and E2a functions, respectively (van Raaij, et al. (1999) Nature 401:935-938). AE1-2a cells were obtained from Michael Kadan (Genetic Therapy, Inc./Novartis, Summit, N.J.) and maintained in Improved Modified Eagle Medium (IMEM; Mediatech, Herndon, Va.) containing 10% FBS, 200 μg/ml Hygromycin B (Calbiochem, San Diego, Calif.) and 200 μg/ml Neomycin sulfate (Calbiochem).

Growth of the fiber-deleted viruses in packaging cells that express a fiber protein as well as complementing the E1 deletion allows generation of particles with any desired fiber.

Packaging Cell Lines

Packaging cell lines were generated by stably transfecting expression constructs for the fibers of interest (Von Seggern et al., J. Virol. 74:354-362 (2000)) into an A549-derived E1- and E2a-complementing cell line (Gorziglia et al., J. Virol. 70:4173-4178 (1996)), and clones that expressed the fibers at high levels were selected.

Cell Lines Expressing Ad5 or Ad37 Fiber.

AE1-2a S8 cells were electroporated as previously described (Von Seggern et al. (1998) J. Gen Virol, 79: 1461-1468) with pDV67 (as described above; see also U.S. Application Ser. No. 60/459,000) and stable lines were selected with zeocin (600 μg/ml) to produce cell lines expressing the Ad5 fiber. The Ad5 fiber-expressing cells were designated cell line 633. Similarly, cell lines expressing the Ad37 fiber were produced by electroporating AE1-2a cells with plasmid pDV121 and stable lines were selected with zeocin (800 μg/ml). Cell lines expressing the Ad37 fiber were designated cell lines 761, 762 and 763.

AE1-2a-derived cell lines 633 and 761, expressing the Ad5 (Von Seggern, et al., (1999) J. Virol. 73:1601-1608) and Ad37 (Wu et al., (2001) Virology 279: 78-89) fiber proteins, respectively, were maintained in IMEM, 10% FBS, 200 μg/ml Hygromycin B, 200 μg/ml Neomycin sulfate, and 300 μg/ml Zeocin (Invitrogen, Carlsbad, Calif.).

Cell Lines Expressing Chimeric Fibers.

7 μg of the fiber expression vectors, pLP13, 23, and 32, were stably transfected into 5×10⁶ AE1-2a cells suspended in IMEM, 0.1 mM DTT using a Gene Pulser II (Bio-Rad, Richmond, Calif.) at 0.3 kV and 500 μF. Cells were plated overnight in growth medium and selected using 600 μg/ml Zeocin, 400 μg/ml Hygromycin B, and 400 μg/ml Neomycin sulfate. Selected colonies were analyzed by immunofluorescence, using anti-fiber monoclonal 4D2 antibody (NeoMarkers, Fremont, Calif.) and AlexaFluor® 488 goat anti-mouse IgG conjugate (Molecular Probes, Eugene, Oreg.). pLP43 was transiently transfected into 293T cells using SuperFect transfection reagent (Qiagen, Valencia, Calif.) as described by manufacturer's instructions.

### EXAMPLE 3

### Construction of Fiberless Ad5 Particles

To construct the E1/fiber deleted viral vector, pDV44 is prepared as described herein. Plasmid pDV44 is derived from pBHG10, a vector prepared as described by Bett et al., (1994) Proc. Natl. Acad. Sci., USA, 91:8802-8806, now described in International Application Publication No. WO 95/00655, with methodology known to one of skill in the art. This plasmid also is commercially available from Microbix (Toronto, Canada). It contains an Ad5 genome with the packaging signals at the left end deleted and the E3 region (nucleotides 28133-30818) replaced by a linker with a unique site for the restriction enzyme PacI. An 11.9 kb BamHI fragment, which contains the right end of the adenovirus genome, is isolated from pBHG10 and cloned into the BamHI site of pBS/SK(+) to create plasmid p11.3 having approximately 14,658 bp. The p11.3 plasmid was then digested with PacI and SalI to remove the fiber, E4, and inverted terminal repeat (ITR) sequences.

This fragment was replaced with a 3.4 kb fragment containing the ITR segments and the E4 gene, which was generated by PCR amplification from pBHG10 using the following oligonucleotide sequences: 5′ TGTACACCG GATCCGGCGCACACC3′ SEQ ID No. 25; and 5′CACAACGAGCTC AATTAATTAATTGCCACATCCTC3′ SEQ ID No. 26. These primers incorporated sites for PacI and BamHI. Cloning this fragment into the PacI and blunt ended SalI sites of the p11.3 backbone resulted in a substitution of the fused ITRs, E4 region and fiber gene present in pBHG10, by the ITRs and E4 region alone. The resulting p11.3 plasmid containing the ITR and E4 regions, designated plasmid pDV43a, was then digested with BamHI. This BamHI fragment was then used to replace a BamHI fragment in pBHG10 thereby creating pDV44 in a pBHG10 backbone.

In an alternative approach, pDV44 was prepared using an additional subcloning step to facilitate the incorporation of restriction cloning sites. This alternative cloning procedure was performed as follows. pDV44 as above was constructed by removing the fiber gene and some of the residual E3 sequences from PBHG10 (Microbix Biosystems). As above, to simplify manipulations, the 11.9 kb BamHI fragment including the rightmost part of the Ad5 genome was removed from pBHG10 and inserted into pBS/SK. The resulting plasmid was termed p11.3. The 3.4 kb DNA fragment corresponding to the E4 region and both ITRs of adenovirus type 5 was amplified as described above from pBHG10 using the oligonucleotides listed above and subcloned into the vector pCR2.1 (Invitrogen) to create pDV42. This step is the additional cloning step to facilitate the incorporation of a SalI restriction site. pDV42 was then digested with PacI, which cuts at a unique site in one of the PCR primers, and with SalI, which cuts at a unique site in the pCR2.1 polylinker. This fragment was used to replace the corresponding PacI/XhoI fragment of p11.3 (the pBS polylinker adjacent to the Ad DNA fragment contains a unique XhoI site), creating pDV43.

Ad5.GFP.ΔF was constructed by recombination in bacteria using a modification of the AdEasy System (see, U.S. Pat. No. 5,922,576 and He et al. (1998) Proc. Natl. Acad. Sci. U.S.A. 95:2509-2514; the system is publicly available from the authors at Johns Hopkins University and other sources). First, a fiber-deleted genomic plasmid was constructed by removing the fiber gene from pAdEasy-1. Plasmid pAdEasy-1 contains the entire Ad5 genome, except for nucleotides 1-3,533, which encompass the E1 genes, and nucleotides 28, 130-30,820, which encompass the E3 gene.

Plasmid pDV43 was digested with PacI, the ends blunted by treatment with the large fragment of E. coli DNA polymerase and dNTPs, and the product re-ligated to produce plasmid pDV76. The resulting plasmid pDV76 is identical to pDV43 except for loss of the PacI site and contains the right end of the Ad5 genome with E3 and fiber deletions. A 4.23 kb fragment from PDV76 was amplified using the oligonucleotide primers (SEQ ID NOS: 27 and 28, respectively): 5′CGC GCT GAC TCT TAA GGA CTA GTT TC 3′, including the unique SpeI site in the Ad5 genome (bold); and 5′ GCG CTT AAT TAA CAT CAT CAA TAA TAT ACC TTA TTT T 3′, including a new PacI site (bold) adjacent to the right Ad5 ITR. The resulting PCR amplified fragment contains nucleotides 27,082 to 35,935 of the Ad5 genome with deletions of nucleotides 28,133 to 32,743 (the E3 and fiber genes), and was used to replace the corresponding Spe1/Pac1 fragment of pAdEasy 1 (see, U.S. Pat. No. 5,922,576) to create pDV77.

Second, E. coli strain BJ5183 was electroporated with a mixture of pDV77 and Pme1-linearized pAdTrack as described (U.S. Pat. No. 5,922,576; He et al. (1998) Proc. Natl. Acad. Sci. U.S.A. 95:2509-2514), and DNA was isolated from kanamycin-resistant colonies. The resulting plasmid, pDV83, contains a complete Ad5 genome with E1-, E3-, and fiber-deletions with a CMV-driven GFP reporter gene inserted at the site of the E1 deletion.

The full length Ad chromosome was isolated by PacI digestion, and transfected into the E1- and fiber-complementing 633 cells described herein (see also, Von Seggern et al. (2000) J. Virol. 74:354-362). The recovered virus Ad5.GFP.ΔF was then plaque purified by plating on 633 cells and virus stocks were prepared by freeze-thawing cell pellets.

### EXAMPLE 4

### Construction of Pseudotyped Viruses

Ad5-Pseudotype Particle Production

A system for testing modified fiber genes to identify tropisms of interest is described in copending U.S. application Ser. No. 09/482,682 (published as US20030157688; see, also as International PCT application No. PCT/US00/00265). The in vitro system involves infection of tissue culture cells with a fiber-deleted Ad and transfection with a plasmid directing fiber expression. This system allows one to produce and evaluate modified fibers expressed on a viral particle. This system can be used to produce therapeutic quantities of adenoviral vectors with modified fiber proteins

Ad5.GFP.ΔF/5F and Ad5.GFP.ΔF/37F pseudotyped Ad5 vectors were produced by propagating Ad5.GFP.ΔF in fiber complementing 633 and 761 cells, respectively. The particles produced by growth in the various cell lines are identical except for their fiber proteins.

Particles with Ad5 Fiber

Ad5-pseudotyped particles (Ad5.GFP.ΔF/5F) were generated by virus growth in 633 cells, which express the wild type Ad5 fiber protein. Viral particles were isolated and purified by centrifugation on preformed 15-40% CsCl gradients (111,000×g for three hours at 4° C.; Von Seggern et al. (1999) J. Virol. 73:1601-16080).

Particles with Ad37 Fiber

Cells from the Ad37 fiber producing cell line 761 were infected at approximately 1000 particles/cell with Ad5.GFP.ΔF. Viral particles were isolated and purified over CsCl gradients, as described above. The bands were harvested, dialyzed into storage buffer (10 mM Tris-pH 8.1, 0.9% NaCl, and 10% glycerol), aliquoted and stored at −70° C.

Particles with Chimeric Fibers

Cells expressing either the Ad37s/Ad5k, Ad5s/Ad37k, Ad5As or Ad5s/Ad37s fibers as described herein were infected at 75-80% confluency with Ad5.GFP.ΔF/5F at approximately 2000 particles per cell. Cells were detached around 72 hours post-infection and lysed by repeated freezing and thawing. Cell debris was removed by centrifugation, and the liberated virus particles were purified in a 16-40% CsCl gradient at 111,000×g for 3 hours. Purified virus was dialyzed into TBS, 10% glycerol. Protein concentration was determined by a Bradford protein assay (Bio-Rad). Virus concentration was calculated from the protein concentration using the known molecular weight of Ad2 particles (1 μg=4×10⁹ particles).

### EXAMPLE 5

### Fiber Expression Assay

Immunooblot analyses of Ad particles was used to analyze fiber expression. Five hundred nanograms of virus was denatured by boiling in a 2% sodium dodecyl sulfate (SDS) and 0.2 M 2-mercaptoethanol buffer for 5 minutes. Viral proteins were separated by SDS-polyacrylamide gel electrophoresis (SDS-PAGE) in an 8-16% Tris-GIycine gel (Novex/Invitrogen) and transferred to a polyvinyl difluoride (PVDF) membrane. The membrane was blocked in 5% (w/v) milk in phosphate buffered saline, 0.02% (v/v) Tween-20 (PBS-T) overnight at 4° C. After blocking, the membrane was incubated with 4D2 anti-fiber monoclonal antibody (NeoMarkers, Fremont, Calif.) diluted 1:1,000 in milk in PBS-T for 1 hour at room temperature. The membrane was washed and incubated with 1:10,000 goat anti-mouse horseradish peroxidase conjugated antibody (Sigma-Aldrich, St. Louis, Mo.) for 30 minutes at room temperature. After washing the membrane again, the blot was probed with enhanced chemiluminescence reagents (Supersignal West Pico reagents; Pierce, Rockford, Ill.) and developed on film.

To ensure equal loading of virus samples, the membrane was stripped and reprobed for penton base. The membrane was incubated with 100 mM 2-mercaptoethanol, 2% SDS, 62.5 mM Tris, pH 6.7 for 1 hour at 42° C. After being washed, the membrane was probed with 1:500 dilution of a rabbit anti-penton polyclonal antibody (Wickham et al. (1993) Cell 73:309-319) for 1 hour, washed, probed with 1:5,000 goat anti-rabbit HRP conjugated antibody (Sigma-Aldrich) for 30 minutes, and washed again. The penton blot was developed as described above.

Each of the chimeric or truncated fiber-pseudotyped Ad particles contained similar amounts of fiber protein of the expected size. A comparison of the fiber immunoblots with penton base blots showed that similar amounts of each virus were analyzed.

### EXAMPLE 6

### Fiber Structure: Length and Rigidity Assessment

Cryo-EM and single particle image reconstruction methods were used to visualize the Ad5s/Ad37s fiber protein incorporated into an Ad5 pseudotyped virus particle. Small droplets (3 μl) of a purified viral preparation of fiber pseudotyped Ad (Ad5s/Ad37s) (˜200 ug/ml) were applied to glow-discharged holey carbon grids. The grids were blotted and vitrified in ethane slush chilled by liquid nitrogen (Adrian, et al., (1984) Nature 308:32-36). The frozen grids were transferred one at a time to a Gatan 626 cryo-transfer holder pre-chilled with liquid nitrogen or stored under liquid nitrogen. Electron micrographs were recorded using low dose conditions on a FEI/Philips CM120 transmission electron microscope equipped with a LaB₆ filament and a Gatan slow-scan CCD camera (YAG scintillator, 1024 by 1024 pixels). A nominal magnification of 35,000× was used, yielding a pixel size of 5.2 Å on the molecular scale. Images were collected with defocus values of −1.5, −1.0 and −0.7 μm to generate phase contrast.

Particles were digitally selected from cryo-electron micrographs in 360 by 360 pixel image files using the QVIEW software package (Shah, et al., (1998) J. Struct. Biol. 123:17-21) and most of the further image processing was done using the IMAGIC-5 software package (van Heel, et al., (1996) J. Struct. Biol. 116:17-2440). Initial particle orientations were obtained using a previous reconstruction of Ad5 as the search model (van Raaij, et al., (1999) Nature 401:935-93842). Once the initial orientations were obtained, each particle was reconstructed separately to determine if its fibers were straight enough to generate significant reconstructed fiber density. If the reconstruction based on a single Ad particle showed fiber density above the background noise level and along more than 50% of the predicted fiber length, it was selected for inclusion in the data set of Ad particles with the straightest fibers. In this manner, 85 Ad particle images were selected from a total set of 1,236 particle images of the fiber-pseudotyped Ad (Ad5s/Ad37s). Seven out of 403 wild-type Ad5 particle images were selected using the same criteria. Computational correction for the contrast transfer function (CTF) of the electron microscope was done prior to merging particle images collected with different defocus values as described in Chiu et al, (1999) J. Virol. 73:6759-6768.

Incomplete correction of the CTF most likely explains why the fiber density is apparently disconnected from the penton base. Four rounds of anchor set refinement were performed with the selected set of particle images using a 1° angular search step size. This improved the resolution of the reconstructed capsid from 37 to 29 Å. Although the icosahedral capsid density improved, the reconstructed fiber density did not improve with refinement. This observation implies that even the straightest fibers must not all be perfectly aligned along the icosahedral 5-fold axes. The unrefined reconstruction based on the selected 85 particle images were selected.

The resolution of each reconstruction was assessed by the Fourier shell correlation method using the 0.5 correlation threshold criterion. “Soft” masks were applied to the two half-reconstructions in order to consider just the icosahedral capsid in the resolution assessment (Stewart, et al., (2000) Microsc Res Tech 49:224-232). All of the image processing and graphics were performed on Compaq/DEC alpha workstations. The graphics representations were generated with the AVS-5 software package (Advanced Visualization Systems, Inc., Waltham Mass.).

In order to determine the significance of these findings, a statistical chi square analysis was performed. This test indicated a very small probability, 0.0001, that the null hypothesis is true, i.e. that the fiber pseudotyped Ad particles and wild-type Ad5 particles have equally flexible fibers. A statistically significant number of pseudotyped Ad particles with Ad5s/Ad37s fibers are found with reasonably ‘straight’ fibers as compared with Ad5 wild-type particles. The cryo-EM analysis that the Ad5s/Ad37s fibers are less flexible than the wild-type Ad5 fibers and that the chimeric fibers probably can not bend with as large an angle as wild-type.

In this combined reconstruction fiber density is apparent out to 359±18 Å from the penton base, which is close to the 331±5 Å reported for the Ad2 fiber in intact penton (Ruigrok, et al., (1990) J. Mol. Biol. 215:589-596). The 5% error range in the length measurement is due to the error range of the microscope magnification value. The measured fiber length is roughly equivalent to that expected for the full length Ad5 fiber.

### EXAMPLE 7

### Infection Assay

Infection assays using pseudotyped Ad vectors carrying a GFP transgene were performed. Briefly, 50,000 adherent A549 cells were incubated with 20,000 particles per cell vector for 3 hours at 37° C. in DMEM, 10% FBS. Cells were washed three times with saline and cultured overnight in growth medium. Cells were detached and analyzed by fluorescence-assisted cell sorting (FACS) in a FACScan cytometer (Becton Dickenson, Franklin Lakes, N.J.). A threshold established by the fluorescence of uninfected cells was used to distinguish infected cells expressing GFP.

The results of the experiments show that Ad particles equipped with the Ad5 wild type fiber exhibited ˜8 fold higher infection than viruses equipped with the Ad37 fiber. Particles displaying the Ad37 shaft fused to the Ad5 knob (Ad37s/Ad5k) or an Ad5 fiber lacking 14 repeats in the central shaft domain (Ad5Δs) had significantly reduced infectivity, demonstrating that the fiber shaft domain plays a crucial role in cell infection. Further evidence was observed by placing the Ad37 knob on the Ad5 fiber shaft (Ad5s/Ad37k). This construct increased virus infectivity nearly to the level of wild type Ad5 fibers. The enhanced infectivity can be abolished by the addition of an excess of anti-CAR antibody (data not shown). Thus, these findings demonstrate that the repeats in the Ad5 fiber shaft are important for cell infection by Ad particles.

Ad5 particles equipped with the chimeric Ad5s/Ad37s fiber were tested for the ability to support Ad infection. The results show that replacing the 3^{rd} and 21^{st} β-repeats of Ad5 with the corresponding regions in the more rigid Ad37 shaft abolished cell infection. Truncated Ad5 fiber (Ad5Δs) also exhibit reduced infectivity. These findings further demonstrate the importance of the 3rd and 21st repeats of the fiber shaft for cell infection.

### EXAMPLE 8

### Virus Attachment Assay

A virus attachment assay was performed to assess whether modifications of the fiber shaft also altered virus attachment to cells.

Cultured A549 cells were detached using 5 mM EDTA for 5 minutes. Cells were resuspended in phosphate-buffered saline (PBS) and aliquoted to a density of 1.0×10⁶ cells per tube. 1.0×10⁹ particles of virus was added to tubes, and the tubes were rocked for 1 hour at 4° C. to prevent internalization. Non-specific Ad binding was determined by the addition of an excess of recombinant Ad5 knob (100 μg/ml). Cells were pelleted by centrifugation and resuspended in PBS three times. Total sample DNA was extracted from cells and bound virus using the QlAamp DNA Mini Kit (Qiagen) as directed by the manufacturer's instructions.

Five μl of each 200 μl DNA extract was added to a 45 μl reaction mixture containing 1× TaqMan Universal PCR Master Mix (Applied Biosystems, Foster City, Calif.), 300 nM 5′ primer EGFP553f (SEQ ID No. 29) and 3′ primer EGFP810r (SEQ ID No. 30), 200 nM probe EGFP734p (SEQ ID No. 31) and VIC-labeled RNase P Control Reagents (Applied Biosystems). EGFP primers and probes were designed to detect a 258 bp region in the EGFP transgene (Klein, et al., (2000) Gene Therapy 7:458-463) in the Ad5 vector genome while RNase P control reagents were designed to amplify a segment of the host cell genomic RNase P gene. After initial denaturation and activation of the AmpliTaq Gold DNA Polymerase by heating to 50° C. for 2 minutes and then 95° C. for 10 minutes, the amplicons were amplified with 40 cycles of 15 seconds at 95° C. followed by 1 minute at 60° C. Fluorescence of reporter dyes FAM and VIC were measured during each cycle in an ABI Prism 7900 Sequence Detection System (Applied Biosystems). Known amounts of pEGFP-N1 plasmid (Clontech, Palo Alto, Calif.), an EGFP expression plasmid, and purified cellular DNA were used as standards to measure the number of copies of Ad genomes and cell number in each sample.

The results of the experiments testing virus attachment assay with pseudotyped virus particles show that Ad5 exhibits higher binding to A549 cells than Ad37 fiber-pseudotyped virus. Particles containing the truncated Ad5 knob (Ad5Δs) or the Ad37 shaft fused to the Ad5 knob (Ad37s/Ad5k) have reduced binding activity; whereas particles equipped with the Ad5 shaft fused to the Ad37 knob (Ad5s/Ad37k) have substantially increased cell attachment. Particles containing a ‘straightened’ Ad5 fiber shaft with the 3rd and 21st repeats from Ad37 (Ad5s/Ad37s) exhibit minimal specific cell attachment demonstrating that these two repeats are important for cell attachment as well as for cell infection. Increased cell attachment of (Ad5s/Ad37k) was competed by the Ad5 knob, indicating restoration of CAR binding since Ad5 infection of A549 cells is CAR-dependent (Wu et al., (2001) Virology 279: 78-89).

### EXAMPLE 9

### Integrin Interaction

Integrin binding to intact virus particles was determined as follows. Purified pseudotyped Ads were coated to wells of a 96-well plate (Immulon 4 HBX; Dynex Technologies, Chantilly, Va.) overnight at room temperature. The wells were blocked with Superblock in PBS (Pierce). Virus-coated wells were first incubated for 2 hrs with varying amounts of soluble αvβ5 integrin (27). After washing, the virus-coated wells were incubated for 1 hr with 10 μg/ml of a non-function-blocking anti-av subunit monoclonal antibody (LM142) (kindly provided by D. Cheresh, TSR1, La Jolla, Calif.). After additional wash steps, the wells were incubated with 1:10,000 goat anti-mouse HRP conjugated antibody for 1 hr. The ELISA was developed with ABTS substrate and analyzed by measuring absorbance at 405 nm.

The results showed that each of the different Ad particles exhibit similar levels of αvβ5 integrin binding indicating that fiber shaft modification does not interfere with the association of these secondary receptors.

### EXAMPLE 10

### Modeling of Receptor Interactions

Recent structural analyses have revealed a striking similarity in the structure of the adenovirus fiber protein and the sigma 1 protein of reovirus (Chappell, et al, (2002) EMBO J. 21:1-117), the attachment protein for the reovirus receptor, JAM-1 (4). CAR and murine JAM (mJAM) share similarities in membrane-distal IgV domains and dimerization interfaces. Both are members of the immunoglobulin superfamily having two Ig-like ectodomains and are both located in tight junctions on host cells (Cohen, et al., (2001) Proc Natl Acad Sci USA 98:15191-15196; Martin-Padura, et al., (1998) J. Cell Biol. 142: 117-127; Wickham (1993) Cell 73:309-319).

Using TOP, a protein topological comparison program (Lu, G. (2000) J. Appl. Cryst. 33:176-183), the crystal structures of mJAM, CAR DI dimer, the Ad12 knob-CAR D1 complex (Bewley, et al., (1999) Science 286:1579-15836) and Ad2 knob plus four β-repeats of the shaft (Raaij, et al., (2000) Structure 8:1147-1155.41) were successively aligned together. Because membrane-distal domains of CAR and mJAM are IgV domains and are 23% identical, their β-strands and secondary structures could be closely aligned. The resultant chimeric CAR-JAM molecule closely resembles the bent cryo-EM structure of human CAR bound to coxsackievirus B3 (He, et al., (2001) Nature Struc. Biol. 8:874-878). Lastly, the 41% identical subgroup C Ad2 knob and shaft, with a nearly identical β-barrel tertiary structure, was easily aligned to the Ad12 knob. The entire fiber-CAR-mJAM complex was manually oriented to the cell surface, assuming that the two molecules from the CAR D1 dimer are equidistant to the planar cell surface. Electron density from a cryo-EM image reconstruction of an Ad5 vector pseudotyped with the Ad37 fiber (Chiu, et al., (2001) J. Virol. 75:5375-5380) was added over the crystal structure of the Ad2 knob and shaft and incorporated in the complex model. In the resulting model of the Ad37-CAR-host cell complex, the angle between the three-fold symmetry axis of the fiber shaft and the cell surface is approximately 20° and, in order to position the CAR binding surface of the Ad37 knob in contact with CAR, the model indicates a significant steric collision between Ad37 and the host cell membrane, with an overlap of roughly 300 Å. Even if there is a moderately large deviation (±300) in the bend angles between the two CAR domains vs. the two mJAM domains, this would not completely alleviate the steric collision predicted between Ad37 and the host cell membrane. In order to avoid steric interference, the rigid Ad37 fiber would have to be oriented such that the angle between the three-fold symmetry axes of the fiber shaft and the cell surface was 60° or greater. This molecular model of the Ad37-CAR-host cell complex based on homology modeling as well as on X-ray crystallographic and cryo-EM structures explains why Ad37 fiber cannot support virus binding via CAR at the cell surface despite containing a CAR-binding sequence in its fiber knob. The steric constraints imposed by the cell surface and receptor orientation, therefore, play a significant role in alignment of fiber and CAR molecules.

1. A modified adenovirus fiber, comprising a modification to the fiber protein shaft, wherein the modification comprises a modification selected from among:
a modification of a last full repeat;
a modification of a β-repeat corresponding to a third β-repeat and a modification of a last full repeat; and
a modification of one or both of a modification of a last full repeat and a modification of at least one amino acid in a contiguous sequence of amino acids corresponding to the amino acid sequence TTVT/S set forth in SEQ ID No. 44 in a third β-repeat, whereby binding of the fiber or of a viral particle containing such fiber to the Coxsackie-Adenovirus Receptor (CAR) is reduced compared to the unmodified fiber.

2. A modified adenovirus fiber, comprising a modification to the fiber protein shaft, whereby binding of the modified fiber to Coxsackie-Adenovirus Receptor (CAR) is reduced or eliminated, wherein:
the unmodified fiber binds the Coxsackie-Adenovirus Receptor (CAR); and
the modification comprises a modification of a repeat corresponding to the last full β-repeat, or the third β-repeat and the last full β-repeat of the shaft or one or both of the last full β-repeat and a portion of the third β-repeat that comprises the TTVT/S motif (SEQ ID No. 44} or a corresponding motif.

3. A modified fiber of claim 1, wherein the modified fiber binds to CAR with less than 50%, 40%, 30%, 20%, 10%, 5%, 1% of the binding affinity of the unmodified fiber.

4. A modified adenovirus fiber of claims 1, wherein the modified fiber is more rigid than the unmodified fiber.

5. A modified adenovirus fiber of claim 1, wherein the modification is a mutation, deletion, insertion or replacement of at least one amino acid in the fiber shaft repeat corresponding to the third repeat.

6. The modified adenovirus fiber of claim 1, wherein the unmodified fiber is a fiber of a serotype C adenovirus.

7. The modified adenovirus fiber of claim 6, wherein the serotype C adenovirus is Ad2 or Ad5.

8-9. (canceled)

10. The modified adenovirus fiber of claim 1, wherein the third β-repeat is modified by replacing it with a corresponding repeat from a serotype D fiber shaft repeat sequence.

11. The modified adenovirus fiber of claim 10, wherein the serotype D adenovirus is selected from the group consisting of Ad8, Ad9, Ad15, Ad19p and Ad37.

12. The modified adenovirus fiber of claim 1, wherein the third β-repeat is modified by replacing it with a corresponding repeat selected from the group consisting of SEQ ID NOS: 58, 66, 67 and 68.

13. The modified adenovirus fiber of claim 1, wherein the modification is a mutation, deletion, insertion or replacement of at least one amino acid in a fiber shaft β-repeat corresponding to the last full β repeat and/or corresponding to the third β-repeat.

14. The modified adenovirus fiber of claim 13, wherein the unmodified fiber is a serotype C adenovirus fiber.

15. The modified adenovirus fiber of claim 12, wherein the serotype C adenovirus is Ad2 or Ad5.

16. The modified adenovirus fiber of claim 15, wherein the modification is a modification of at least one amino acid in a contiguous sequence of amino acids corresponding to those set forth in SEQ ID No. 46 or SEQ ID No. 47.

17. The modified adenovirus fiber of claim 1, wherein the modification comprises replacement of the last full β-repeat with a corresponding repeat sequence from a serotype D adenovirus fiber shaft.

18. The modified adenovirus fiber of claim 17, wherein the serotype D adenovirus is selected from the group consisting of Ad8, Ad9, Ad 15, Ad19p and Ad37.

19. The modified adenovirus fiber of claim 13, wherein the modification is in the last full repeat; and the last full repeat comprises a change of at least one amino acid in the repeat at contiguous amino acids corresponding to the amino acid sequence set forth in SEQ ID No. 49.

20. The modified adenovirus fiber of claim 13, wherein the modification is in the last full repeat; and the last full repeat comprises a sequence of amino acids selected from the group consisting of SEQ ID NOS: 48, 59, 60 and 61.

21. The modified adenovirus fiber of claim 1, wherein a contiguous sequence of amino acids corresponding to the third repeat of the fiber shaft is deleted.

22. The modified adenovirus fiber of any of claim 1, wherein a contiguous sequence of amino acids corresponding to the last full repeat of the fiber shaft is deleted.

23. The modified adenovirus fiber of claim 1, wherein a contiguous sequence of amino acids corresponding to the third repeat and the contiguous sequence of amino acids corresponding to the last full repeat are modified.

24. The modified adenovirus fiber of claim 22, wherein the modification is a mutation, deletion, insertion or replacement of at least one amino acid in a fiber shaft repeat corresponding to the third repeat and/or the last full repeat.

25. The modified adenovirus fiber of claim 24, wherein the unmodified fiber shaft is from a serotype C adenovirus.

26. The modified adenovirus fiber of claim 25, wherein the serotype C adenovirus is Ad2 or Ad5.

27. The modified adenovirus fiber of claim 25, wherein the modified repeats corresponding to the third repeat and the last full repeat are from a serotype D adenovirus.

28. The modified adenovirus fiber of claim 27, wherein the serotype D adenovirus is selected from the group consisting of Ad8, Ad9, Ad15, Ad19p and Ad37.

29. The modified adenovirus fiber of claim 25, wherein the third repeat comprises a sequence selected from the group consisting of SEQ ID NOs. 58, 66, 67 and 68 and the last full repeat comprises an amino acid sequence selected from the group consisting of SEQ ID NOs. 48, 59, 60 and 61.

30. The modified adenovirus fiber of claim 25, wherein the third repeat sequence is selected from a corresponding repeat sequence of a fiber protein from Ad8, Ad9, Ad15, Ad19p or Ad37; and the last full repeat is selected from a corresponding repeat sequence of a fiber protein from Ad8, Ad9, Ad 15, Ad19p or Ad37.

31. The modified adenovirus fiber of claim 1, wherein the modified adenovirus fiber further comprises at least one additional modification in the fiber protein, whereby the modified fiber binds to a receptor other than CAR with greater affinity than the unmodified fiber binds to such receptor.

32. The modified adenovirus fiber of claim 1, wherein the modified adenovirus fiber further comprises at least one additional modification in the fiber protein; and the modification is a modification in the fiber knob that further reduces or eliminates any binding of the modified fiber to CAR.

33. The modified adenovirus fiber of claim 31, wherein an additional modification is a modification of the Heparin Sulfate Proteoglycans (HSP) binding site in the fiber shaft.

34. The modified adenovirus fiber of claim 32, wherein an additional modification is a modification in the fiber knob.

35. The modified fiber of claim 1, wherein the fiber is shortened or its flexibility is reduced compared to the unmodified fiber.

36. The modified adenovirus fiber of claim 34, wherein the fiber knob is replaced with fiber knobs from an adenovirus that does not interact with CAR.

37. The modified adenovirus fiber of claim 36, wherein the adenovirus fiber knob that does not interact with CAR is Ad3 fiber knob, Ad41 short fiber knob, or Ad35 fiber knob.

38. The modified adenovirus fiber of claim 34, wherein fiber knob mutations are mutations in the AB loop or CD loop.

39. The modified adenovirus fiber of claim 38, wherein fiber knob mutations are mutations in the AB loop or CD loop selected from KO1 and KO12.

40. A modified adenovirus fiber, comprising a fiber protein, wherein:
the unmodified fiber binds the Coxsackie-Adenovirus Receptor (CAR);
the fiber protein comprises a modification to the fiber protein shaft such that binding of the modified fiber to CAR is substantially reduced or eliminated;
the modified fiber comprises repeats corresponding to the third repeat and the last full repeat; and at least one repeat of the fiber shaft is deleted.

41. The modified adenovirus fiber of claim 40, wherein the repeats corresponding to repeats 4-17 are deleted.

42. The modified adenovirus fiber of claim 40 or claim 41, wherein the fiber is from a serotype C adenovirus.

43. The modified adenovirus fiber of claim 42, wherein the serotype C adenovirus is Ad2 or Ad5.

44. The modified adenovirus fiber of claim 43, wherein the amino acids corresponding to positions 95-316 are deleted.

45. The modified adenovirus fiber claim 1, wherein the fiber protein is from a serotype A, B, C or F adenovirus; and at least one amino acid corresponding to the consensus repeat sequence as set forth in SEQ ID No. 49 is modified in the repeat corresponding to either the third repeat or the last full repeat.

46. A nucleic acid molecule, comprising a sequence of nucleotides that encodes a modified fiber of any of claim 1.

47. The nucleic acid molecule of claim 46 that comprises a vector.

48. The nucleic acid molecule of claim 46 that comprises heterologous nucleic acid encoding a gene product.

49. The nucleic acid molecule of any of claims 46-4B that is an adenovirus vector.

50. The nucleic acid molecule of claim 49 that is an adenoviral vector from a serotype C adenovirus.

51. The nucleic acid molecule of claim 49, wherein the heterologous nucleic acid encodes a therapeutic product.

52. The nucleic acid molecule of any of claim 46 that is an early generation adenoviral vector, a gutless adenoviral vector or a replication-conditional adenoviral vector.

53. The nucleic acid molecule of claim 52, wherein the replication-conditional adenoviral vector is an oncolytic adenoviral vector.

54. A cell, comprising the nucleic acid of any of claims claim 46.

55-56. (canceled)

57. A cell of claim 54 that is in a packaging cell line.

58. An adenovirus particle, comprising the modified fiber of any of claim 1.

59. The adenovirus particle of claim 58, wherein the capsid further comprises a penton modification.

60. The adenovirus particle of claim 58, wherein the modified fiber includes an N-terminal portion from a fiber of a serotype C Ad virus, wherein the N-terminal portion is sufficient to increase incorporation into the particle compared to in its absence.

61. The adenovirus particle of claim 58, that comprises a modified serotype C genome, wherein the N-terminal portion of the modified fiber comprises at least the N-terminal 15, 16 or 17 amino acids of a serotype C fiber.

62. The particle of claim 61 wherein the serotype C virus is an Ad2 or Ad5 virus.

63. The adenoviral particle of claim 58 that further comprises a targeting ligand in the capsid.

64. The adenovirus particle of claim 58 further, comprising a heterologous nucleic acid in the genome thereof.

65. The adenovirus particle of claim 64, wherein the heterologous nucleic acid encodes a therapeutically effective product.

66. The adenoviral particle of claim 58 that includes a modification to the capsid whereby binding of the viral particle to HSP is altered compared to a particle that expresses an unmodified capsid.

67. The adenoviral particle of claim 66, wherein the capsid modification that alters HSP binding is in the fiber.

68. An adenoviral particle of claim 58, comprising a mutation in the αᵥ integrin-binding region of the capsid, whereby binding to the integrin is eliminated or reduced.

69. The adenoviral particle of claim 58, wherein the fiber further comprises a modification in the fiber knob to further reduce any CAR binding.

70. The adenoviral particle of claim 69, wherein the fiber knob modification is in the AB loop or CD loop.

71. The adenoviral particle of claim 70, wherein the fiber knob modification is selected from the group consisting of K01 and K012.

72. A composition formulated for administration to a subject, comprising the adenovirus particle of claim 58.

73. A method of detargeting an adenoviral vector, comprising reducing or eliminating the binding of an adenoviral particle to CAR by producing an adenoviral particle that expresses a modified fiber of any of claim 1.

74. The method of claim 73, wherein the modified fiber increases the binding to the particular cell type compared to the unmodified fiber.

75. The method of claim 73, wherein the modified fiber comprises at least two modifications such that the binding to a selected cell type is increased relative to the unmodified fiber.

76. The method of claim 75, wherein the second modification comprises the addition of a targeting Ligand in the capsid of the adenoviral particle.

77. The method of claim 75, wherein the second modification comprises the replacement of the fiber knob or a portion thereof.

78. A method, comprising introducing an adenoviral particle of claim 58 into cells; and introducing the cells into a subject.

79. The method of claim 78, wherein the cells are immune cells or fibroblasts.
